(19) United States
(12) Patent Application Publication
(10) Pub. No.: US 2006/0177932 A1
(43) Pub. Date: Aug. 10, 2006

(54) Expansion agents for stem cells

(76) Inventors: Hiromitsu Nakauchi, Minato-ku (JP); Atsushi Iwama, Minato-ku (JP)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/534,386

(22) PCT Filed: Oct. 23, 2003
(86) PCT No.: PCT/JP03/13579

§ 371 Date: Dec. 5, 2005

(30) Foreign Application Priority Data
Nov. 8, 2002 (JP) 2002-326190

(51) Int. Cl. A61K 38/17; C12N 15/867; C12N 5/08

(52) U.S. Cl. 435/456; 435/366; 514/12

(57) ABSTRACT

The present invention relates to a method and substance for maintaining the pluripotency and self-replication ability of a stem cell, such as a hematopoietic stem cell, while keeping it undifferentiated. Specifically, the present invention provides a composition for maintaining the expansion or pluripotency of a stem cell, comprising active STAT5, and a method using the same. STAT5 may be in the form of a protein or a nucleic acid. The composition may contain a cellular physiologically active substance (e.g., SCF, TPO, Flt-3L, etc.). The present invention also relates to a cell, a tissue and an organ prepared from the stem cell.

### TECHNICAL FIELD

The present invention relates to a composition, method, and kit for maintaining the expansion, pluripotency (or the level of undifferentiation) or self-replication capability of stem cells. More particularly, the present invention relates to a composition, method, and kit for maintaining the expansion, pluripotency (or the level of undifferentiation) or self-replication capability of stem cells using active STAT5. The present invention also relates to stem cells (particularly, hematopoietic stem cells) prepared by using such a method.

### BACKGROUND ART

Recently, attention has been focused on disease therapy using regenerative medicine (regeneration therapy). However, regeneration therapy has not yet reached a point where it is conventionally applied to a number of patients suffering from organ or tissue dysfunction. To date, a very limited number of such patients have been treated by organ transplantation or use of an auxiliary medical system or apparatus. These therapies have problems in shortage of donors, rejection, infection, durability, and the like. Particularly, the donor shortage raises serious problems. In the case of bone marrow transplantation, bone marrow and umbilical cord blood banks have gradually become more widely used home and abroad, though it is still difficult to provide a limited amount of samples to a number of patients. Therefore, there is an increasing demand for therapies using stem cells and regenerative medicine using the same in order to overcome the above-described problems.

Organs of organisms may be partially lost or seriously injured due to wounds or diseases during their lives. Whether or not damaged organs can be regenerated depends on the organ (or the animal species). In regenerative medicine, an attempt has been made to regenerate organs (or tissues) which cannot naturally undergo regeneration. Regeneration of tissues can be determined by confirming an improvement in the function of the tissue. Mammals have the ability to regenerate tissue and organs to some extent (e.g., regeneration of skin, liver, and blood). However, organs such as heart, lungs, brain, and the like, have poor regenerative ability. Therefore, it has been believed that if the organs are once damaged, their functions cannot be recovered. Conventionally, for example, when an organ is damaged, organ transplantation is substantially the only effective treatment.

It had been presumed since a long time ago that stem cells are present in organs having a high level of regenerative ability. This presumption was proved by experimental bone marrow transplantation using animal models. Subsequent studies have demonstrated that stem cells in bone marrow are resources for regeneration of all kinds of blood cells. It has also been demonstrated that stem cells are present in organs having a high level of regenerative ability, such as bone marrow, skin, and the like. In addition, although it was long believed that the brain cannot be regenerated, it has been demonstrated that stem cells are present in the brain. It has been clarified that stem cells are present in any organ in the body and play a role in regeneration of the organs to some extent. Stem cells present in each tissue have plasticity to an extent beyond expectation, so that stem cells in one organ may be used in regeneration of another organ.

There is a possibility that stem cells can be used for regeneration of organs which have not been possible to date. Regenerative medicine, particularly stem cell therapy, has increasingly attracted attention. Recently, as needs for regeneration research are increased in the field of medicine and therapy, more and more findings on stem cells or organ formation are obtained every year. For example, attention has been focused onto establishment of totipotent embryonic stem cells (ES cells) and preparation of cloned individuals from adult somatic cells. This is because techniques related to development and regeneration can be applied to stem cell therapies. Various fields of regenerative medicine using stem cells have proceeded into preliminary stages for the purpose of clinical applications. In some fields, stem cell therapies have been in actual use.

In regenerative medicine, it is most important to reconstruct organs. There are roughly two methods for reconstructing organs: organs are reconstructed ex vivo; and organs are reconstructed in vivo. In either case, stem cells are required for reconstruction of organs. Pluripotency and self-replication ability are importantly required for stem cells for used in the above-described application.

Stem cells are roughly divided into two categories: embryonic stem cells and somatic (tissue) stem cells. Among somatic stem cells, hematopoietic stem cells have attracted attention since a long time ago. The self-replication ability and pluripotency of hematopoietic stem cells are required for maintaining mature blood cells having a short life time during the life-span of a human. This has already been proposed in 1961 (Till, J. E., et al., Radiat. Res. 14: 213-222). In 1972, a bone marrow transplantation method using a mouse model was established (Micklem, H. S., et al.: J. Cell Physiol., 79: 293-298, 1972), thereby making it possible to detect hematopoietic stem cells by investigating reconstruction of the hematopoietic stem system. Since then, hematopoietic stem cell research has made dramatic progress. Subsequent studies revealed the presence of hematopoietic stem cells having self-replication ability and pluripotency (Dick, J. E., et al.; Cell 42; 71-79, 1985). However, hematopoietic stem cells are present at a low rate of several in about 100,000 bone marrow cells even in the case of mice. Therefore, in actual research or clinical application, hematopoietic stem cells need to be concentrated or purified.

Conventionally, hematopoietic stem cell transplantation therapies have been carried out using naturally-occurring cells, leading to various side effects. For example, a side effect (RRT) is produced by pretreatment before transplantation using a large dose of an anticancer drug or radiation. There are also other side effects as follows: bacterial or fungal infectious diseases and hemorrhage due to suppression of bone marrow; in the case of allotransplantation, when donor's leucocytes survive and the number of the cells is increased, they recognize recipient organs as foreign matter and attack them (graft versus host diseases (GVHD)); various pulmonary complications, mainly including cytomegalovirus (CMV) pneumonia; various visceral disorders due to disorders of vascular endothelial cells (cells lining the inner wall of blood vessels); various infectious diseases during immune suppression prolonged after survival of transplanted cells (at least 1-2 years); prolonged chronic GVHD exhibiting various symptoms; late-onset disorders (e.g., secondary cancers, gonad dysfunction, infertility, etc.); and the like.

Due to the above-described complications, transplantation often temporarily worsens systemic conditions. The rate of death of patients due to complications is about 10 to 20% in autotransplantation and about 20 to 40% in allotransplantation. Even if patients overcome complications, some patients may relapse. Thus, the current transplantation therapies are inadequate.

To prevent early death due to complications after bone marrow transplantation, a therapeutic method has been developed in which stem cells are separated and purified and precursor cells are produced in large quantities from the stem cells, and the precursor cells as well as the stem cell are transplanted. The method has already entered clinical trials.

Fluorescence activated cell sorting (FACS) was developed in the 1980's. Since then, techniques utilizing FACS have been employed for enrichment and purification of hematopoietic stem cells. It has been revealed that high-purity hematopoietic stem cells are obtained by separating CD34-KSL cells from multiply stained bone marrow cells (Osawa, M. et al., Science, 273: 242-245, 1996). As described above, pluripotency and self-replication ability are the essential features of stem cells. To exploit these abilities, it is important to enrich and purify stem cells and expand the cells by in vitro culture.

Various proteins have an important role in regulating the expansion and differentiation of stem cells. For example, stem cell factor (SCF) (also called steel factor) in hematopoietic stem cells has attracted attention.

SCF is produced by bone marrow stromal cells and acts on pluripotent stem cells, bone marrow cells (e.g., CFU-M, CFU-GM, CFU-Meg, etc.), and lymphocyte precursor cells to support their expansion and differentiation. That is, it is believed that SCF acts on cells from hematopoietic stem cells to precursor cells so as to aid other cytokines which induce differentiation toward the final stage (S. Kitamura, Saitokain-no-Saizensen [Frontline of Cytokine], Yodo-sha, edited by T. Hirano, pp. 174-187, 2000).

However, the action of a sole SCF seems to be so weak that it cannot work well unless it operates in cooperation with other factors. For example, SCF induces the differentiation and expansion of hematopoietic stem cells strongly in the presence of other cytokines, such as interleukin IL-3, IL-6, IL-11, granulocyte colony stimulating factor (G-CSF), or the like. SCF also induces the differentiation and expansion of mast cells, erythroblast precursor cells, granulocyte macrophage precursor cells, megakaryocyte precursor cells, and the like.

Therefore, it is considered that SCF does not directly control expansion and differentiation, but enhances the responsiveness of a number of kinds of hematopoietic cells to various cytokines while supporting the survival of the cells.

Thrombopoietin (TPO) has also attracted attention. This factor supports the differentiation of megakaryocytes and the production of platelets as well as acting on stem cells to induce their expansion and differentiation. Also, it has been found that TPO is involved in the self replication of stem cells.

Thus, conventional factors can promote the differentiation of stem cells in an uncontrollable manner, but not in a controllable manner.

It has been reported that signals transduced via thrombopoietin receptors and gp130 are effective for maintenance of the undifferentiated state and self-replication of hematopoietic stem cells. It is known that various factors, such as a JAK-Stat system and the like, are present downstream of the signal. Substantially no specific factor has been clarified which plays a role in maintenance of the undifferentiated state and/or self-replication of stem cells.

Therefore, an object of the present invention is to provide a method and substance for expanding a stem cell, such as a hematopoietic stem cell, or maintaining the pluripotency and/or self-replication ability of a stem cell.

### DISCLOSURE OF THE INVENTION

The present invention provides a solution to the above-described problem based on, in part, the present inventors' finding that active STAT5 has a unexpected function of maintaining the pluripotency and self-replication ability of a stem cell, such as a hematopoietic stem cell, while keeping the cell undifferentiated.

Thus, the present invention provides the following.

(1) A composition for maintaining the expansion, pluripotency, or self-replication ability of a stem cell, comprising active STAT5.

(2) A composition according to item 1, wherein the stem cell is a hematopoietic stem cell.

(3) A composition according to item 1, wherein the active STAT5 is active STAT5A or active STAT5B.

(4) A composition according to item 1, wherein the active STAT5 is active STAT5A.

(5) A composition according to item 1, wherein the active STAT5 is:

(a) a polypeptide consisting of an amino acid sequence set forth in SEQ ID NO:2, 4, 6 or 8, or a fragment thereof;

(b) a polypeptide comprising an amino acid sequence set forth in SEQ ID NO:2, 4, 6 or 8 having at least one mutation selected from at least one amino acid substitution, addition and deletion, and having biological activity;

(c) a polypeptide encoded by an allelic variant of a base sequence set forth in SEQ ID NO:1, 3, 5 or 7;

(d) a polypeptide which is a species homolog of an amino acid sequence set forth in SEQ ID NO:2, 4, 6 or 8; or

(e) a polypeptide having an amino acid sequence having at least 70% identity to any one of the polypeptides (a) to (d), and having biological activity; and

wherein in the active STAT5, at least one serine, threonine, or tyrosine residue is phosphorylated.

(6) A composition according to item 1, wherein the active STAT5 is a homodimer or a heterodimer of:

(a) a polypeptide consisting of an amino acid sequence set forth in SEQ ID NO:2, 4, 6 or 8, or a fragment thereof;

(b) a polypeptide comprising an amino acid sequence set forth in SEQ ID NO:2, 4, 6 or 8 having at least one mutation selected from at least one amino acid substitution, addition and deletion, and having biological activity;

(c) a polypeptide encoded by an allelic variant of a base sequence set forth in SEQ ID NO:1, 3, 5 or 7;

(d) a polypeptide which is a species homolog of an amino acid sequence set forth in SEQ ID NO:2, 4, 6 or 8; or

(e) a polypeptide having an amino acid sequence having at least 70% identity to any one of the polypeptides (a) to (d), and having biological activity.

(7) A composition according to item 1, wherein in the active STAT5, a tyrosine residue at position 694 in SEQ ID NO:2 or a tyrosine residue corresponding thereto is at least phosphorylated.

(8) A composition according to item 1, wherein the active STAT5 is a dimer.

(9) A composition according to item 1, wherein the active STAT5 has an amino acid residue substitution in at least one position selected from the group consisting of position, 150, 298, and 710 in SEQ ID NO:2 or 6.

(10) A composition according to item 1, wherein in the active STAT5, a histidine residue at position 298 and/or a serine residue at position 710 in SEQ ID NO:2 or 6, or residues corresponding thereto, are substituted with arginine and/or phenylalanine, respectively.

(11) A composition according to item 1, wherein in the active STAT5, a glutamic acid residue at position 150 and/or a serine residue at position 710 in SEQ ID NO:2 or 6, or residues corresponding thereto, are substituted with glycine and/or phenylalanine, respectively.

(12) A composition according to item 1, wherein in the active STAT5, a serine residue at position 710 in SEQ ID NO:2 or 6 is substituted with phenylalanine.

(13) A composition according to item 1, wherein the active STAT5 has an amino acid sequence set forth in SEQ ID NO:10 or 13.

(14) A composition according to item 1, wherein the active STAT5 is consistently active or transiently active.

(15) A composition according to item 1, further comprising a cellular physiologically active substance.

(16) A composition according to item 15, wherein the cellular physiologically active substance is selected from the group consisting of SCF, TPO, and Flt-3L.

(17) A composition according to item 15, wherein the cellular physiologically active substance contains SCF, TPO, and Flt-3L.

(18) A composition according to item 1, further comprising a pharmaceutically acceptable carrier.

(19) A composition for maintaining the expansion, pluripotency, or self-replication ability of a stem cell, comprising STAT5 and an agent capable of activating STAT5.

(20) A composition according to item 19, wherein the STAT5 is:

(a) a polypeptide consisting of an amino acid sequence set forth in SEQ ID NO:2, 4, 6 or 8, or a fragment thereof;

(b) a polypeptide comprising an amino acid sequence set forth in SEQ ID NO:2, 4, 6 or 8 having at least one mutation selected from at least one amino acid substitution, addition and deletion, and having biological activity;

(c) a polypeptide encoded by an allelic variant of a base sequence set forth in SEQ ID NO:1, 3, 5 or 7;

(d) a polypeptide which is a species homolog of an amino acid sequence set forth in SEQ ID NO:2, 4, 6 or 8; or

(e) a polypeptide having an amino acid sequence having at least 70% identity to any one of the polypeptides (a) to (d), and having biological activity.

21. A composition according to item 19, wherein the STAT5 activating agent is a member selected from the JAK family or a variant thereof.

(22) A composition for maintaining the expansion, pluripotency, or self-replication ability of a stem cell, comprising a nucleic acid molecule encoding active STAT5.

(23) A composition according to item 22, wherein the nucleic acid molecule encoding active STAT5 contains a nucleic acid sequence encoding STAT5 which forms a dimer.

(24) A composition according to item 22, wherein the nucleic acid molecule encoding active STAT5 is:

(a) a polynucleotide having a base sequence set forth in SEQ ID NO:1, 3, 5 or 7 or a sequence fragment thereof;

(b) a polynucleotide encoding a polypeptide consisting of an amino acid sequence set forth in SEQ ID NO:2, 4, 6 or 8, or a fragment thereof;

(c) a polynucleotide encoding a variant polypeptide having an amino acid sequence set forth in SEQ ID NO:2, 4, 6 or 8 having at least one mutation selected from the group consisting of at least one amino acid substitution, addition and deletion, and having biological activity;

(d) a polynucleotide which is an allelic variant of DNA consisting of a base sequence set forth in SEQ ID NO:2, 4, 6 or 8;

(e) a polynucleotide encoding a species homolog of a polypeptide consisting of an amino acid sequence set forth in SEQ ID NO:2, 4, 6 or 8;

(f) a polynucleotide encoding a polypeptide hybridizable to any one of the polynucleotides (a) to (e) under stringent conditions, and having biological activity; or

(g) a polynucleotide consisting of a base sequence having at least 70% identity to any one of the polynucleotides (a) to (e) or a complementary sequence thereof, and having biological activity.

(25) A composition according to item 22, wherein the active STAT5 has an amino acid residue substitution in at least one position selected from the group consisting of position, 150, 298, and 710 in SEQ ID NO:2 or 6.

(26) A composition according to item 22, wherein in the active STAT5, a histidine residue at position 298 and/or a serine residue at position 710 in SEQ ID NO:2 or 6, or residues corresponding thereto, are substituted with arginine and/or phenylalanine, respectively.

(27) A composition according to item 22, wherein in the active STAT5, a glutamic acid residue at position 150 and/or a serine residue at position 710 in SEQ ID NO:2 or 6, or residues corresponding thereto, are substituted with glycine and/or phenylalanine, respectively.

(28) A composition according to item 22, wherein in the active STAT5, a serine residue at position 710 in SEQ ID NO:2 or 6 is substituted with phenylalanine.

(29) A composition according to item 22, wherein the active STAT5 has an amino acid sequence set forth in SEQ ID NO:10 or 13.

(30) A composition according to item 22, wherein the nucleic acid molecule is contained in a vector.

(31) A composition according to item 22, wherein the nucleic acid molecule is contained in a retrovirus vector.

(32) A composition according to item 22, wherein the nucleic acid molecule has a sequence set forth in SEQ ID NO:9.

(33) A composition for expanding a stem cell, comprising a nucleic acid molecule encoding STAT5 and an agent capable of activating the STAT5.

(34) A composition according to item 33, wherein the nucleic acid molecule encoding active STAT5 is:

(a) a polynucleotide having a base sequence set forth in SEQ ID NO:1, 3, 5 or 7 or a sequence fragment thereof;

(b) a polynucleotide encoding a polypeptide consisting of an amino acid sequence set forth in SEQ ID NO:2, 4, 6 or 8, or a fragment thereof;

(c) a polynucleotide encoding a variant polypeptide having an amino acid sequence set forth in SEQ ID NO:2, 4, 6 or 8 having at least one mutation selected from the group consisting of at least one amino acid substitution, addition and deletion, and having biological activity;

(d) a polynucleotide which is an allelic variant of DNA consisting of a base sequence set forth in SEQ ID NO:2, 4, 6 or 8;

(e) a polynucleotide encoding a species homolog of a polypeptide consisting of an amino acid sequence set forth in SEQ ID NO:2, 4, 6 or 8;

(f) a polynucleotide encoding a polypeptide hybridizable to any one of the polynucleotides (a) to (e) under stringent conditions, and having biological activity; or

(g) a polynucleotide consisting of a base sequence having at least 70% identity to any one of the polynucleotides (a) to (e) or a complementary sequence thereof, and having biological activity.

(35) A composition according to item 33, wherein the nucleic acid molecule is contained in a vector.

(36) A composition according to item 33, wherein the nucleic acid molecule is contained in a retrovirus vector.

(37) A composition according to item 33, wherein the STAT5 activating agent is a member selected from the JAK family or a variant thereof.

(38) A method for maintaining the expansion, pluripotency, or self-replication ability of a stem cell, comprising the steps of:

A) providing a stem cell; and

B) providing active STAT5 to the stem cell.

(39) A method for maintaining the expansion, pluripotency, or self-replication ability of a stem cell, comprising the steps of:

A) providing a stem cell;

B) providing STAT5 to the stem cell; and

C) activating the STAT5.

(40) A method for preparing a stem cell in a predetermined quantity, comprising the steps of:

A) providing a stem cell; and

B) providing active STAT5, or STAT5 and an agent capable of activating the STAT5, to the stem cell.

(41) A cell, obtained by a method according to any one of items 38 to 40.

(42) Use of active STAT5 for maintaining the expansion, pluripotency, or self-replication ability of a stem cell.

(43) Use of STAT5 for maintaining the expansion, pluripotency, or self-replication ability of a stem cell.

(44) Use of STAT5 and an agent capable of activating the STAT5 for maintaining the expansion, pluripotency, or self-replication ability of a stem cell.

(45) A cell, obtained by treating a stem cell with active STAT5, or STAT5 and an agent capable of activating the STAT5.

(46) A tissue, obtained by a cell obtained by treating a stem cell with active STAT5, or STAT5 and an agent capable of activating the STAT5.

(47) An organ, obtained by a cell obtained by treating a stem cell with active STAT5, or STAT5 and an agent capable of activating the STAT5.

(48) A medicament composition, comprising a cell obtained by treating a stem cell with active STAT5, or STAT5 and an agent capable of activating the STAT5.

(49) A method for treatment or prophylaxis of a disease or a disorder in need of a stem cell or a differentiated cell derived therefrom, comprising the steps of:

A) administering to a subject in need of the treatment or prophylaxis a cell obtained by treating a stem cell with active STAT5, or STAT5 and an agent capable of activating the STAT5.

(50) Use of a cell obtained by treating a stem cell with active STAT5, or STAT5 and an agent capable of activating the STAT5, for treatment or prophylaxis of a disease or a disorder in need of a stem cell or a differentiated cell derived therefrom.

(51) Use of a cell obtained by treating a stem cell with active STAT5, or STAT5 and an agent capable of activating the STAT5, for production of a medicament for treatment or prophylaxis of a disease or a disorder in need of a stem cell or a differentiated cell derived therefrom.

These and other advantages of the present invention will become apparent to those skilled in the art upon reading and understanding the following detailed description with reference to the accompanying figures.

### BRIEF DESCRIPTION OF THE DRAWINGS

FIG. 1 is a diagram illustrating a self-replication signal in hematopoietic stem cells.

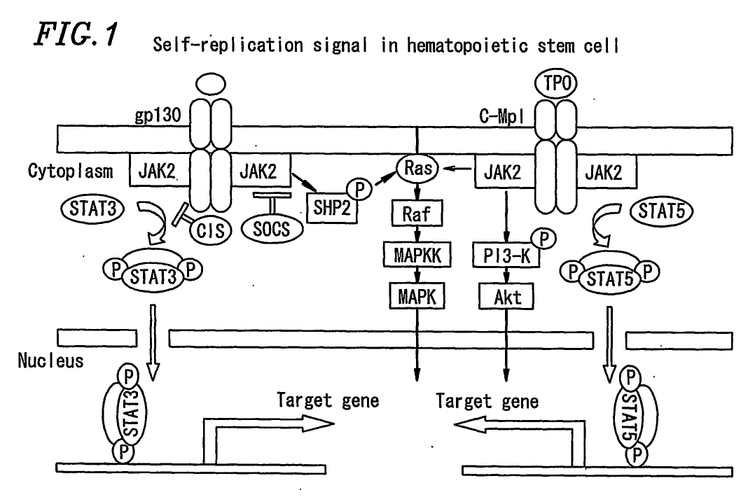

FIG. 2 is a diagram illustrating a structure of consistently active STAT5 used in the present invention.

FIG. 3 is a diagram illustrating a protocol for a colony assay used in the present invention.

FIG. 4 is a diagram showing a growth curve.

FIG. 5 is a diagram showing a time schedule of an experiment used in the present invention.

FIG. 6 is a diagram showing the results of the colony assay.

FIG. 7 is a graph based on the results of FIG. 6, where MIX indicates colonies containing myelocytes, erythroblasts, and megakaryocytes, and GM/M indicates colonies containing only myelocytes.

FIG. 8 is a diagram showing a time schedule of bone marrow transplantation.

FIG. 9 is a diagram showing the results of a bone marrow transplantation experiment.

### DESCRIPTION OF THE SEQUENCE LISTING

SEQ ID NO:1 is a human nucleic acid sequence of STAT5A.

SEQ ID NO:2 is a human amino acid sequence of STAT5A.

SEQ ID NO:3 is a human nucleic acid sequence of STAT5B.

SEQ ID NO:4 is a human amino acid sequence of STAT5B.

SEQ ID NO:5 is a mouse nucleic acid sequence of STAT5A.

SEQ ID NO:6 is a mouse amino acid sequence of STAT5A.

SEQ ID NO:7 is a mouse nucleic acid sequence of STAT5B.

SEQ ID NO:8 is a mouse amino acid sequence of STAT5B.

SEQ ID NO:9 is a nucleic acid sequence of STAT5A 1*6, which is a variant of STAT5A.

SEQ ID NO:10 is an amino acid sequence of STAT5A 1*6, which is a variant of STAT5A.

SEQ ID NO:11 is a consensus sequence of STAT5.

SEQ ID NO:12 is a nucleic acid sequence of STAT5A 1*7, which is a variant of STAT5A.

SEQ ID NO:13 is an amino acid sequence of STAT5A 1*7, which is a variant of STAT5A.

### BEST MODE FOR CARRYING OUT THE INVENTION

Hereinafter, the present invention will be described. It should be understood throughout the present specification that articles for singular forms (e.g., “a”, “an”, “the”, etc. in English; “ein”, “der”, “das”, “die”, etc. and their inflections in German; “un”, “une”, “le”, “la”, etc. in French; “un”, “una”, “el”, “la”, etc. in Spanish, and articles, adjectives, etc. in other languages) include plural referents unless the context clearly dictates otherwise. It should be also understood that the terms as used herein have definitions typically used in the art unless otherwise mentioned. If there is contradiction, the present specification (including the definition) takes precedence.

Terms particularly used herein are defined as follows.

(Expansion Agent)

As used herein, the term “STAT5” (signal transducer and activator of transcription 5) refers to an agent having a sequence as set forth in SEQ ID NO:1, 3, 5 or 7 (nucleic acid sequence) or 2, 4, 6 or 8 (amino acid sequence) and corresponding agents (orthologs) in other species of animals. STAT5 can be identified by detecting nuclear transfer ability and ability to bind to a specific DNA sequence, and further transcription factor activity. STAT5 has a SH2 (src homology 2) domain which recognizes a specific phosphorylated structure. When a serine residue, threonine residue, or tyrosine residue of STAT5 is phosphorylated, STAT5 is dimerized into homodimers, which are in turn transferred into nuclei. The homodimer recognizes and binds to a specific DNA sequence to control transcription of a number of genes. For STAT5, it appears particularly important that tyrosine at position 694 in SEQ ID NO:2 or 6 is phosphorylated. As used herein, such a portion that is specifically recognized by STAT5 is called “STAT5 consensus sequence”. The sequence has a consensus sequence

5′-GATCCGAATTCCAGGAATTCAGATC-3′. (SEQ ID NO:11)

It is known that homologs of STAT5 are present in mammals, such as human, rat, mouse, and the like, and fruit fly, and the like. Therefore, as used herein, STAT5 typically refers to STAT5 present in mammals and other general organisms.

It is known that STAT5 includes STAT5A and STAT5B, which are considerably similar to each other, in mammals and the like. It will be appreciated in the present invention that if STAT5A and STAT5B are present, the molecules both have substantially the same function. In a certain preferred embodiment, STAT5A may be employed.

As used herein, the term “active STAT5” refers to a molecule obtained by activating STAT5, which has ability to transfer into nuclei (nuclear transfer ability) and ability to bind to the STAT5 consensus sequence. The active STAT5 is characterized in that at least one serine, threonine or tyrosine residue thereof is phosphorylated. Representatively, the term “active STAT5” refers to a dimmer of STAT5s in both of which a tyrosine residue outside the SH2 domain is phosphorylated. In a non-limiting example, such a tyrosine residue is at position 694 in SEQ ID NO:2 or 6.

The active STAT5 can be artificially or synthetically produced. An example of such an artificially produced STAT5 is STAT5 which is mutated so as to be consistently dimerized. Examples of STAT5 capable of consistent dimerization, i.e., consistently active STAT5, include, but are not limited to, a molecule having a sequence set forth in SEQ ID NO:10 or 13.

In another embodiment, active STAT5 may be a low molecular weight compound (e.g., a product of a combinatorial library, etc.). Such a low molecular weight compound can be easily screened for by those skilled in the art. Such screening can be carried out by using an assay for measuring the activity of active STAT5 as described herein.

As used herein, active STAT5 may be any molecule as long as it has the same function as that of naturally-occurring active STAT5 (e.g., dimerized phosphorylated STAT5A). Whether or not an agent is active STAT5 can be determined by detecting the possession of ability to transfer into cell nuclei and/or the possession of ability to bind to the STAT5 consensus sequence. Specifically, when the agent is a nucleic acid molecule, nuclear transfer can be detected by transfecting cells with a STAT5 gene and immunologically staining the cells using anti-STAT5 antibodies to confirm the localization in the nuclei. When the agent is a protein or the like, nuclear transfer can be detected by introducing the agent directly into cells and thereafter immunologically staining the cells using antibodies specific to the agent. These techniques are well known in the art.

To detect ability to bind to the STAT5 consensus sequence, a gel shift assay is employed, in which a double-stranded oligonulceotide comprising a base sequence (5′-GATCCGAATTCCAGGAATTCAGATC-3′) (SEQ ID NO:11) containing a prolactin responsive element (PRE) as a STAT5 binding base sequence is used. A nuclear extract of cells transfected with a certain agent (in the case of a nucleic acid) or the agent itself (in the case of a protein or the like) is reacted with a nucleic acid molecule comprising the base sequence, followed by polyacrylamide gel electrophoresis and separation. The binding ability can be detected by identifying the formation of a complex of the agent or a gene product of the agent with a nucleic acid molecule comprising the consensus sequence. Typically, if the formation of a complex can be significantly confirmed, the agent can be determined to have the same function as that of active STAT5.

Transcription activation is detected using a bovine β casein promoter comprising the PRE sequence as described above. A luciferase gene is linked as a reporter gene downstream of the β casein promoter. Cells are simultaneously transfected with the reporter gene and a STAT5 gene. Thereafter, the luciferase activity is measured. Alternatively, when the agent is applied directly to the gene, cells are transfected with only the reporter gene and the luciferase activity is measured. Thereby, whether or not a molecule of interest is active STAT5 can be determined. If the luciferase activity is increased, the molecule can be recognized as having transcription activation ability. While not wishing to be bound by the following, the recognition can be made by performing statistical processing and determining the statistical significance.

Whether or not a molecule of interest is active STAT5 can be sufficiently determined by detecting a significant level of activity using at least one of the above-described methods. If activity is confirmed by a luciferase reporter assay, it is indirectly proved that the molecule is transferred into nuclei and binds to DNA. Therefore, the determination can be basically made by observing a signal from the luciferase reporter.

STAT5 plays a significant role in a signal transduction system for thrombopoietin. STAT5 is found in hematopoietic stem cells. It is considered that STAT5 plays a role in hematopoietic stem cells. It is known that JAK2/STAT5 is activated via thrombopoietin to transfer various signals, such as proliferation, differentiation, suppression of cell death, and the like, into immune cells (e.g., T-cells, B-cells, and the like), hematopoietic cells, liver cells, and neural cells. Therefore, in the present invention, substantially the same effect can be achieved by stimulating a signal transduction pathway involved in production of active STAT5, such as thrombopoietin and the like.

As used herein, the term “consistent” in relation to active STAT5 indicates that a function of the active STAT5 is maintained unless it is stimulated. In contrast, the term “transient” in relation to active STAT5 indicates that a function of the active STAT5 is exhibited during a certain period of time. In a non-limiting example, consistent active STAT5 is one which is always formed as a dimer, such as STAT5A 1*6. Examples of consistent active STAT5 include, but are not limited to, STAT5A 1*6 (SEQ ID NO:9 and 10, in which histidine at position 298 and serine at position 710 are substituted with arginine and phenylalanine, respectively, in SEQ ID NO:6), and variants having corresponding sequences in species homologs (e.g., human variants and the like).

A sugar chain may be added to active STAT5 used herein at a portion, such as a portion to which N-acetyl-D-glucosamine can bind via an N-glucoside bond and a portion to which N-acetyl-D-galactosamine can bind via an O-glucoside bond (a portion in which a serine or a threonine residue frequently appears). The activity of STAT5 or active STAT5 used herein is not particularly affected by the presence or absence of a sugar chain. However, proteins having an added sugar chain are typically stable in organisms against decomposition and may have strong physiological activity. Therefore, polypeptides having an added sugar chain are within the scope of the present invention.

(Biochemistry and Molecular Biology)

The terms “protein”, “polypeptide”, “oligopeptide” and “peptide” as used herein have the same meaning and refer to an amino acid polymer having any length. This polymer may be a straight, branched or cyclic chain. An amino acid may be a naturally-occurring or nonnaturally-occurring amino acid, or a variant amino acid. The term may include those assembled into a complex of a plurality of polypeptide chains. The term also includes a naturally-occurring or artificially modified amino acid polymer. Such modification includes, for example, disulfide bond formation, glycosylation, lipidation, acetylation, phosphorylation, or any other manipulation or modification (e.g., conjugation with a labeling moiety). This definition encompasses a polypeptide containing at least one amino acid analog (e.g., nonnaturally-occurring amino acid, etc.), a peptide-like compound (e.g., peptoid), and other variants known in the art, for example. In specific cases, the term “polypeptide” as used herein may refers to STAT5 or active STAT5.

The terms “polynucleotide”, “oligonucleotide”, and “nucleic acid” as used herein have the same meaning and refer to a nucleotide polymer having any length. This term also includes an “oligonucleotide derivative” or a “polynucleotide derivative”. An “oligonucleotide derivative” or a “polynucleotide derivative” includes a nucleotide derivative, or refers to an oligonucleotide or a polynucleotide having different linkages between nucleotides from typical linkages, which are interchangeably used. Examples of such an oligonucleotide specifically include 2′-O-methyl-ribonucleotide, an oligonucleotide derivative in which a phosphodiester bond in an oligonucleotide is converted to a phosphorothioate bond, an oligonucleotide derivative in which a phosphodiester bond in an oligonucleotide is converted to a N3′-P5′ phosphoroamidate bond, an oligonucleotide derivative in which a ribose and a phosphodiester bond in an oligonucleotide are converted to a peptide-nucleic acid bond, an oligonucleotide derivative in which uracil in an oligonucleotide is substituted with C-5 propynyl uracil, an oligonucleotide derivative in which uracil in an oligonucleotide is substituted with C-5 thiazole uracil, an oligonucleotide derivative in which cytosine in an oligonucleotide is substituted with C-5 propynyl cytosine, an oligonucleotide derivative in which cytosine in an oligonucleotide is substituted with phenoxazine-modified cytosine, an oligonucleotide derivative in which ribose in DNA is substituted with 2′-O-propyl ribose, and an oligonucleotide derivative in which ribose in an oligonucleotide is substituted with 2′-methoxyethoxy ribose. Unless otherwise indicated, a particular nucleic acid sequence also implicitly encompasses conservatively-modified variants thereof (e.g. degenerate codon substitutions) and complementary sequences as well as the sequence explicitly indicated. Specifically, degenerate codon substitutions may be produced by generating sequences in which the third position of one or more selected (or all) codons is substituted with mixed-base and/or deoxyinosine residues (Batzer et al., Nucleic Acid Res. 19:5081(1991); Ohtsuka et al., J. Biol. Chem. 260:2605-2608 (1985); Rossolini et al., Mol. Cell. Probes 8:91-98(1994)).

As used herein, the term “nucleic acid” is also used interchangeably with the terms “gene”, “cDNA”, “mRNA”, “oligonucleotide”, and “polynucleotide”. A particular nucleic acid sequence includes a “splice variant”. Similarly, a particular protein encoded by a nucleic acid implicitly encompasses any protein encoded by a splice variant of that nucleic acid. “Splice variants”, as the name suggests, are products of alternative splicing of a gene. After transcription, an initial nucleic acid transcript may be spliced such that different (alternative) nucleic acid splice products encode different polypeptides. Mechanisms for the production of splice variants vary, but include alternative splicing of exons. Alternative polypeptides derived from the same nucleic acid by read-through transcription are also encompassed by this definition. In the present invention, STAT5 may be in the form of a nucleic acid.

As used herein, “gene” refers to an element defining a genetic trait. A gene is typically arranged in a given sequence on a chromosome. A gene which defines the primary structure of a protein is called a structural gene. A gene which regulates the expression of a structural gene is called a regulatory gene. As used herein, “gene” may refer to “polynucleotide”, “oligonucleotide”, “nucleic acid”, and “nucleic acid molecule” and/or “protein”, “polypeptide”, “oligopeptide” and “peptide”. As used herein, “homology” of a gene refers to the proportion of identity between two or more gene sequences. Therefore, the greater the homology between two given genes, the greater the identity or similarity between their sequences. Whether or not two genes have homology is determined by comparing their sequences directly or by a hybridization method under stringent conditions. When two gene sequences are directly compared with each other, these genes have homology if the DNA sequences of the genes have representatively at least 50% identity, preferably at least 70% identity, more preferably at least 80%, 90%, 95%, 96%, 97%, 98%, or 99% identity with each other.

The similarity, identity and homology of amino acid sequences and base sequences are herein compared using BLAST (sequence analyzing tool) with the default parameters.

As used herein, the term “ligand” refers to a substance capable of specifically binding to a certain protein. Examples of a ligand include lectin, antigens, antibodies, hormones, neurotransmitters, and the like, which are capable of specifically binding to various receptor protein molecules on cell membranes.

As used herein, “polynucleotides hybridizing under stringent conditions” refers to conditions commonly used and well known in the art. Such a polynucleotide can be obtained by conducting colony hybridization, plaque hybridization, southern blot hybridization, or the like using a polynucleotide selected from the polynucleotides of the present invention. Specifically, a filter on which DNA derived from a colony or plaque is immobilized is used to conduct hybridization at 65° C. in the presence of 0.7 to 1.0 M NaCl. Thereafter, a 0.1 to 2-fold concentration SSC (saline-sodium citrate) solution (1-fold concentration SSC solution is composed of 150 mM sodium chloride and 15 mM sodium citrate) is used to wash the filter at 65° C. Polynucleotides identified by this method are referred to as “polynucleotides hybridizing under stringent conditions”. Hybridization can be conducted in accordance with a method described in, for example, Molecular Cloning 2nd ed., Current Protocols in Molecular Biology, Supplement 1-38, DNA Cloning 1: Core Techniques, A Practical Approach, Second Edition, Oxford University Press (1995), and the like. Here, sequences hybridizing under stringent conditions exclude, preferably, sequences containing only A (adenine) or T (thymine). Therefore, the polypeptide of the present invention (e.g., STAT5, active STAT5, or the like) includes a polypeptide encoded by a nucleic acid molecule hybridizable to a nucleic acid molecule encoding a polypeptide particularly described herein under stringent conditions.

As used herein, “Hybridizable polynucleotide” refers to a polynucleotide which can hybridize other polynucleotides under the above-described hybridization conditions. Specifically, the hybridizable polynucleotide includes at least a polynucleotide having a homology of at least 60% to the base sequence of DNA encoding a polypeptide having an amino acid sequence as set forth in SEQ ID NO:2, 4, 6 or 8, preferably a polynucleotide having a homology of at least 80%, and more preferably a polynucleotide having a homology of at least 95%. The homology of a nucleic acid sequence may be represented by similarity evaluated with a score using, for example, a search program BLAST using an algorithm developed by Altschul et al., J. Mol. Biol., 215, 403-410(1990)).

The term “highly stringent conditions” refers to those conditions that are designed to permit hybridization of DNA strands whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.0015 M sodium chloride, 0.0015 M sodium citrate at 65-68° C. or 0.015 M sodium chloride, 0.0015 M sodium citrate, and 50% formamide at 42° C. See Sambrook, Fritsch & Maniatis, Molecular Cloning: A Laboratory Manual (2nd ed., Cold Spring Harbor Laboratory, N.Y., 1989); Anderson et al., Nucleic Acid Hybridization: A Practical Approach Ch. 4 (IRL Press Limited) (Oxford Express). More stringent conditions (such as higher temperature, lower ionic strength, higher formamide, or other denaturing agents) may be optionally used. Other agents may be included in the hybridization and washing buffers for the purpose of reducing non-specific and/or background hybridization. Examples are 0.1% bovine serum albumin, 0.1% polyvinylpyrrolidone, 0.1% sodium pyrophosphate, 0.1% sodiumdodecyl sulfate (NaDodSO₄ or SDS), Ficoll, Denhardt's solution, sonicated salmon sperm DNA (or another non-complementary DNA), and dextran sulfate, although other suitable agents can also be used. The concentration and types of these additives can be changed without substantially affecting the stringency of the hybridization conditions. Hybridization experiments are ordinarily carried out at pH 6.8-7.4; however, at typical ionic strength conditions, the rate of hybridization is nearly independent of pH. See Anderson et al , Nucleic Acid Hybridization: A Practical Approach Ch. 4 (IRL Press Limited, Oxford UK).

Agents affecting the stability of DNA duplex include base composition, length, and degree of base pair mismatch. Hybridization conditions can be adjusted by those skilled in the art in order to accommodate these variables and allow DNAs of different sequence relatedness to form hybrids. The melting temperature of a perfectly matched DNA duplex can be estimated by the following equation:
Tm (° C.)=81.5+16.6 (log[Na⁺])+0.41 (% G+C)−600/N−0.72 (% formamide)
where N is the length of the duplex formed, [Na⁺] is the molar concentration of the sodium ion in the hybridization or washing solution, % G+C is the percentage of (guanine+cytosine) bases in the hybrid. For imperfectly matched hybrids, the melting temperature is reduced by approximately 1° C. for each 1% mismatch.

The term “moderately stringent conditions” refers to conditions under which a DNA duplex with a greater degree of base pair mismatching than could occur under “highly stringent conditions” is able to form. Examples of typical “moderately stringent conditions” are 0.015 M sodium chloride, 0.0015 M sodium citrate at 50-65° C. or 0.015 M sodium chloride, 0.0015 M sodium citrate, and 20% formamide at 37-50° C. By way of example, “moderately stringent conditions” of 50° C. in 0.015 M sodium ion will allow about a 21% mismatch.

It will be appreciated by those skilled in the art that there is no absolute distinction between “highly stringent conditions” and “moderately stringent conditions”. For example, at 0.015 M sodium ion (no formamide), the melting temperature of perfectly matched long DNA is about 71° C. With a wash at 65° C. (at the same ionic strength), this would allow for approximately a 6% mismatch. To capture more distantly related sequences, those skilled in the art can simply lower the temperature or raise the ionic strength.

A good estimate of the melting temperature in 1 M NaCl for oligonucleotide probes up to about 20 nucleotides is given by:
Tm=(2° C. per A-T base pair)+(4° C. per G-C base pair).

Note that the sodium ion concentration in 6× salt sodium citrate (SSC) is 1 M. See Suggs et al., Developmental Biology Using Purified Genes 683 (Brown and Fox, eds., 1981).

As used herein, the term “isolated” biological agent (e.g., nucleic acid, protein, or the like) refers to a biological agent that is substantially separated or purified from other biological agents in cells of a naturally-occurring organism (e.g., in the case of nucleic acids, agents other than nucleic acids and a nucleic acid having nucleic acid sequences other than an intended nucleic acid; and in the case of proteins, agents other than proteins and proteins having an amino acid sequence other than an intended protein). The “isolated” nucleic acids and proteins include nucleic acids and proteins purified by a standard purification method. The isolated nucleic acids and proteins also include chemically synthesized nucleic acids and proteins.

As used herein, the term “purified” biological agent (e.g., nucleic acids, proteins, and the like) refers to one from which at least a part of naturally accompanying agents is removed. Therefore, ordinarily, the purity of a purified biological agent is higher than that of the biological agent in a normal state (i.e., concentrated).

As used herein, the terms “purified” and “isolated” mean that the same type of biological agent is present preferably at least 75% by weight, more preferably at least 85% by weight, even more preferably at least 95% by weight, and most preferably at least 98% by weight.

As used herein, the term “expression” of a gene, a polynucleotide, a polypeptide, or the like, indicates that the gene or the like is affected by a predetermined action in vivo to be changed into another form. Preferably, the term “expression” indicates that genes, polynucleotides, or the like are transcribed and translated into polypeptides. In one embodiment of the present invention, genes may be transcribed into mRNA. More preferably, these polypeptides may have post-translational processing modifications. In a preferred embodiment, such an expressed polypeptide may be consistently or transiently activated STAT5.

As used herein, the term“interaction” with reference to two substances means that one substance influences the other substance via forces (e.g., intermolecular forces (Van der Waals force), hydrogen bonding, hydrophobic interactions, or the like). Typically, two substances interacting with each other interact in the manner of association or binding.

As used herein, the term “binding” means the physical or chemical interaction between two proteins or compounds or associated proteins or compounds or combinations thereof. Binding includes ionic, non-ionic, hydrogen, Van der Waals, hydrophobic interactions, etc. A physical interaction (binding) can be either direct or indirect. Indirect interactions may be through or due to the effects of another protein or compound. Direct binding refers to interactions that do not take place through, or due to, the effect of another protein or compound, but instead are without other substantial chemical intermediates.

As used herein, the term “modulate” or “modify” refers to an increase or decrease or maintenance in a specific activity, or the amount, quality or effect of a protein.

A naturally-occurring nucleic acid encoding a protein (e.g., STAT5, or variants or fragments thereof, or the like) may be readily isolated from a cDNA library having PCR primers and hybridization probes containing part of a nucleic acid sequence indicated by, for example, SEQ ID NO: 1 or the like. A preferable nucleic acid encoding STAT5, or variants or fragments thereof, or the like is hybridizable to the whole or part of a sequence as set forth in SEQ ID NO:1 or 3 under low stringent conditions defined by hybridization buffer essentially containing 1% bovine serum albumin (BSA); 500 mM sodium phosphate (NaPO₄); 1 mM EDTA; and 7% SDS at 42° C., and wash buffer essentially containing 2×SSC (600 mM NaCl; 60 mM sodium citrate); and 0.1% SDS at 50° C., more preferably under low stringent conditions defined by hybridization buffer essentially containing 1% bovine serum albumin (BSA); 500 mM sodium phosphate (NaPO₄); 15% formamide; 1 mM EDTA; and 7% SDS at 50° C., and wash buffer essentially containing 1×SSC (300 mM NaCl; 30 mM sodium citrate); and 1% SDS at 50° C., and most preferably under low stringent conditions defined by hybridization buffer essentially containing 1% bovine serumalbumin (BSA); 200 mM sodium phosphate (NaPO₄); 15% formamide; 1 mM EDTA; and 7% SDS at 50° C., and wash buffer essentially containing 0.5×SSC (150 mM NaCl; 15 mM sodium citrate); and 0.1% SDS at 65° C.

As used herein, the term “probe” refers to a substance for use in searching, which is used in a biological experiment, such as in vitro and/or in vivo screening or the like, including, but not being limited to, for example, a nucleic acid molecule having a specific base sequence or a peptide containing a specific amino acid sequence.

Examples of a nucleic acid molecule as a common probe include one having a nucleic acid sequence having a length of at least 8 contiguous nucleotides, which is homologous or complementary to the nucleic acid sequence of a gene of interest. Such a nucleic acid sequence may be preferably a nucleic acid sequence having a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 25 contiguous nucleotides, a length of at least 30 contiguous nucleotides, a length of at least 40 contiguous nucleotides, or a length of at least 50 contiguous nucleotides. A nucleic acid sequence used as a probe includes a nucleic acid sequence having at least 70% homology to the above-described sequence, more preferably at least 80%, and even more preferably at least 90% or at least 95%.

As used herein, the term “search” indicates that a given nucleic acid sequence is utilized to find other nucleic acid base sequences having a specific function and/or property either electronically or biologically, or using other methods. Examples of an electronic search include, but are not limited to, BLAST (Altschul et al. , J. Mol. Biol. 215:403-410 (1990)), FASTA (Pearson & Lipman, Proc. Natl. Acad. Sci., USA 85:2444-2448 (1988)), Smith and Waterman method (Smith and Waterman, J. Mol. Biol. 147:195-197 (1981) and Needleman and Wunsch method (Needleman and Wunsch, J. Mol. Biol. 48:443-453 (1970)), and the like. Examples of a biological search include, but are not limited to, a macro array in which genomic DNA is attached to a nylon membrane or the like or a microarray (microassay) in which genomic DNA is attached to a glass plate under stringent hybridization, PCR and in situ hybridization, and the like. It is herein intended that STAT5 and the like used in the present invention include corresponding genes identified by such an electronic or biological search.

As used herein, the “percentage of sequence identity, homology or similarity (amino acid, nucleotide, or the like)” is determined by comparing two optimally aligned sequences over a window of comparison, wherein the portion of a polynucleotide or polypeptide sequence in the comparison window may comprise additions or deletions (i.e. gaps), as compared to the reference sequences (which does not comprise additions or deletions (if the other sequence includes an addition, a gap may occur)) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e. the window size) and multiplying the results by 100 to yield the percentage of sequence identity. When used in a search, homology is evaluated by an appropriate technique selected from various sequence comparison algorithms and programs well known in the art. Examples of such algorithms and programs include, but are not limited to, TBLASTN, BLASTP, FASTA, TFASTA and CLUSTALW (Pearson and Lipman, 1988, Proc. Natl. Acad. Sci. USA 85 (8):2444-2448, Altschul et al., 1990, J. Mol. Biol. 215(3) :403-410, Thompson et al., 1994, Nucleic Acids Res. 22(2) :4673-4680, Higgins et al., 1996, Methods Enzymol. 266:383-402, Altschul et al., 1990, J. Mol. Biol. 215(3) :403-410, Altschul et al., 1993, Nature Genetics 3:266-272). In a particularly preferable embodiment, the homology of a protein or nucleic acid sequence is evaluated using a Basic Local Alignment Search Tool (BLAST) well known in the art (e.g., see Karlin and Altschul, 1990, Proc. Natl. Acad. Sci. USA 87:2267-2268, Altschul et al., 1990, J. Mol. Biol. 215:403-410, Altschul et al., 1993, Nature Genetics 3:266-272, Altschul et al., 1997, Nuc. Acids Res. 25:3389-3402). Particularly, 5 specialized-BLAST programs may be used to perform the following tasks to achieve comparison or search:
(1) comparison of an amino acid query sequence with a protein sequence database using BLASTP and BLAST3;
(2) comparison of a nucleotide query sequence with a nucleotide sequence database using BLASTN;
(3) comparison of a conceptually translated product in which a nucleotide query sequence (both strands) is converted over 6 reading frames with a protein sequence database using BLASTX;
(4) comparison of all protein query sequences converted over 6 reading frames (both strands) with a nucleotide sequence database using TBLASTN; and
(5) comparison of nucleotide query sequences converted over 6 reading frames with a nucleotide sequence database using TBLASTX.

The BLAST program identifies homologous sequences by specifying analogous segments called “high score segment pairs” between amino acid query sequences or nucleic acid query sequences and test sequences obtained from preferably a protein sequence database or a nucleic acid sequence database. A large number of the high score segment pairs are preferably identified (aligned) using a scoring matrix well known in the art. Preferably, the scoring matrix is the BLOSUM62 matrix (Gonnet et al., 1992, Science 256:1443-1445, Henikoff and Henikoff, 1993, Proteins 17:49-61). The PAM or PAM250 matrix may be used, although they are not as preferable as the BLOSUM62 matrix (e.g., see Schwartz and Dayhoff, eds., 1978, Matrices for Detecting Distance Relationships: Atlas of Protein Sequence and Structure, Washington: National Biomedical Research Foundation). The BLAST program evaluates the statistical significance of all identified high score segment pairs and preferably selects segments which satisfy a threshold level of significance independently defined by a user, such as a user set homology. Preferably, the statistical significance of high score segment pairs is evaluated using Karlin's formula (see Karlin and Altschul, 1990, Proc. Natl. Acad. Sci. USA 87:2267-2268).

As used herein, the term “primer” refers to a substance required for initiation of a reaction of a macromolecule compound to be synthesized, in a macromolecule synthesis enzymatic reaction. In a reaction for synthesizing a nucleic acid molecule, a nucleic acid molecule (e.g., DNA, RNA, or the like) which is complementary to part of a macromolecule compound to be synthesized may be used.

A nucleic acid molecule which is ordinarily used as a primer includes one that has a nucleic acid sequence having a length of at least 8 contiguous nucleotides, which is complementary to the nucleic acid sequence of a gene of interest. Such a nucleic acid sequence preferably has a length of at least 9 contiguous nucleotides, more preferably a length of at least 10 contiguous nucleotides, even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 16 contiguous nucleotides, a length of at least 17 contiguous nucleotides, a length of at least 18 contiguous nucleotides, a length of at least 19 contiguous nucleotides, a length of at least 20 contiguous nucleotides, a length of at least 25 contiguous nucleotides, a length of at least 30 contiguous nucleotides, a length of at least 40 contiguous nucleotides, and a length of at least 50 contiguous nucleotides. A nucleic acid sequence used as a primer includes a nucleic acid sequence having at least 70% homology to the above-described sequence, more preferably at least 80%, even more preferably at least 90%, and most preferably at least 95%. An appropriate sequence as a primer may vary depending on the property of the sequence to be synthesized (amplified). Those skilled in the art can design an appropriate primer depending on the sequence of interest. Such primer design is well known in the art and may be performed manually or using a computer program (e.g., LASERGENE, Primer Select, DNAStar).

As used herein, the term “amino acid” may refer to a naturally-occurring or nonnaturally-occurring amino acid. The term “amino acid derivative” or “amino acid analog” refers to an amino acid which is different from a naturally-occurring amino acid and has a function similar to that of the original amino acid. Such amino acid derivatives and amino acid analogs are well known in the art.

The term “naturally-occurring amino acid” refers to an L-isomer of a naturally-occurring amino acid. The naturally-occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine, and lysine. Unless otherwise indicated, all amino acids as used herein are L-isomers.

The term “nonnaturally-occurring amino acid” refers to an amino acid which is ordinarily not found in nature. Examples of nonnaturally-occurring amino acids include D-form of amino acids as described above, norleucine, para-nitrophenylalanine, homophenylalanine, para-fluorophenylalanine, 3-amino-2-benzyl propionic acid, D- or L-homoarginine, and D-phenylalanine. The term “amino acid analog” refers to a molecule having a physical property and/or function similar to that of amino acids, but is not an amino acid. Examples of amino acid analogs include, for example, ethionine, canavanine, 2-methylglutamine, and the like. An amino acid mimic refers to a compound which has a structure different from that of the general chemical structure of amino acids but which functions in a manner similar to that of naturally-occurring amino acids.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “corresponding” amino acid or nucleic acid refers to an amino acid or nucleotide in a given polypeptide or polynucleotide molecule, which has, or is anticipated to have, a function similar to that of a predetermined amino acid or nucleotide in a polypeptide or polynucleotide as a reference for comparison. Particularly, in the case of enzyme molecules, the term refers to an amino acid which is present at a similar position in an active site and similarly contributes to catalytic activity. For example, in the case of antisense molecules, the term refers to a similar portion in an ortholog corresponding to a particular portion of the antisense molecule. In the active STAT5 of the present invention, a corresponding amino acid may be a phosphorylated site, for example. In another embodiment, in the active STAT5 of the present invention, a corresponding amino acid may be an amino acid playing a role in dimerization. Such a “corresponding” amino acid or nucleic acid may extend over a region or domain having a certain range. Therefore, in this case, such a region or domain is herein referred to as a “corresponding” region or domain.

As used herein, the term “corresponding” gene (e.g., a polypeptide or polynucleotide molecule) refers to a gene (e.g., a polypeptide or polynucleotide molecule) in a given species, which has, or is anticipated to have, a function similar to that of a predetermined gene in a species as a reference for comparison. When there are a plurality of genes having such a function, the term refers to a gene having the same evolutionary origin. Therefore, a gene corresponding to a given gene may be an ortholog of the given gene. Therefore, genes corresponding to mouse STAT5A and STAT5B genes and the like can be found in other animals (human, rat, pig, cattle, and the like). Such a corresponding gene can be identified by techniques well known in the art. Therefore, for example, a corresponding gene in a given animal can be found by searching a sequence database of the animal (e.g., human, rat) using the sequence of a reference gene (e.g., mouse STAT5A and STAT5B genes, and the like) as a query sequence.

As used herein, the term “nucleotide” may be either naturally-occurring or nonnaturally-occurring. The term “nucleotide derivative” or “nucleotide analog” refers to a nucleotide which is different from naturally-occurring nucleotides and has a function similar to that of the original nucleotide. Such nucleotide derivatives and nucleotide analogs are well known in the art. Examples of such nucleotide derivatives and nucleotide analogs include, but are not limited to, phosphorothioate, phosphoramidate, methylphosphonate, chiral-methylphosphonate, 2-O-methyl ribonucleotide, and peptide-nucleic acid (PNA).

As used herein, the term “fragment” refers to a polypeptide or polynucleotide having a sequence length ranging from 1 to n−1 with respect to the full length of the reference polypeptide or polynucleotide (of length n). The length of the fragment can be appropriately changed depending on the purpose. For example, in the case of polypeptides, the lower limit of the length of the fragment includes 3, 4, 5, 6, 7, 8, 9, 10, 15, 20, 25, 30, 40, 50 or more nucleotides. Lengths represented by integers which are not herein specified (e.g., 11 and the like) may be appropriate as a lower limit. For example, in the case of polynucleotides, the lower limit of the length of the fragment includes 5, 6, 7, 8, 9, 10, 15, 20, 25, 30, 40, 50, 75, 100 or more nucleotides. Lengths represented by integers which are not herein specified (e.g., 11 and the like) may be appropriate as a lower limit.

As used herein, the term “specifically interact with” indicates that a first substance or agent interacts with a second substance or agent with higher affinity than that to substances or agents other than the second substance or agent (particularly, other substances or agents in a sample containing the second substance or agent). Examples of a specific interaction with reference to a substance or agent include, but are not limited to, hybridization of nucleic acids, antigen-antibody reaction, ligand-receptor reaction, enzyme-substrate reaction, a reaction between a transcriptional agent and a binding site of the transcriptional agent when both a nucleic acid and a protein are involved, a protein-lipid interaction, a nucleic acid-lipid interaction, and the like. Therefore, when the first and second substances or agents are nucleic acids, “specifically interact with” means that the first substance or agent is at least partially complementary to the second substance or agent. Alternatively, when both the first and second substances or agents are proteins, “specifically interact with” includes, but is not limited to, an interaction due to antigen-antibody reaction, an interaction due to receptor-ligand reaction, an enzyme-substrate interaction, and the like. When the two substances or agents are a protein and a nucleic acid, “specifically interact with” includes an interaction between a transcriptional agent and a binding region of a nucleic acid molecule targeted by the transcriptional agent. As used herein, the term “agent capable of specifically interacting with” a biological agent, such as a polynucleotide, a polypeptide or the like, refers to an agent which has an affinity to the biological agent, such as a polynucleotide, a polypeptide or the like, which is representatively higher than or equal to an affinity to other non-related biological agents, such as polynucleotides, polypeptides or the like (particularly, those with identity of less than 30%), and preferably significantly (e.g., statistically significantly) higher. Such an affinity can be measured with, for example, a hybridization assay, a binding assay, or the like. As used herein, the “agent” may be any substance or other agent (e.g., energy, such as light, radiation, heat, electricity, or the like) as long as the intended purpose can be achieved. Examples of such a substance include, but are not limited to, proteins, polypeptides, oligopeptides, peptides, polynucleotides, oligonucleotides, nucleotides, nucleic acids (e.g., DNA such as cDNA, genomic DNA, or the like, and RNA such as mRNA), polysaccharides, oligosaccharides, lipids, low molecular weight organic molecules (e.g., hormones, ligands, information transfer substances, molecules synthesized by combinatorial chemistry, low molecular weight molecules (e.g., pharmaceutically acceptable low molecular weight ligands and the like), and the like), and combinations of these molecules. Examples of an agent specific to a polynucleotide include, but are not limited to, representatively, a polynucleotide having complementarity to the sequence of the polynucleotide with a predetermined sequence homology (e.g., 70% or more sequence identity), a polypeptide such as a transcriptional agent binding to a promoter region, and the like. Examples of an agent specific to a polypeptide include, but are not limited to, representatively, an antibody specifically directed to the polypeptide or derivatives or analogs thereof (e.g., single chain antibody), a specific ligand or receptor when the polypeptide is a receptor or ligand, a substrate when the polypeptide is an enzyme, and the like.

As used herein, the term “compound” refers to any identifiable chemical substance or molecule, including, but not limited to, a low molecular weight molecule, a peptide, a protein, a sugar, a nucleotide, or a nucleic acid. Such a compound may be a naturally-occurring product or a synthetic product.

As used herein, the term “low molecular weight organic molecule” refers to an organic molecule having a relatively small molecular weight. Usually, the low molecular weight organic molecule refers to a molecular weight of about 1,000 or less, or may refer to a molecular weight of more than 1,000. Low molecular weight organic molecules can be ordinarily synthesized by methods known in the art or combinations thereof. These low molecular weight organic molecules may be produced by organisms. Examples of the low molecular weight organic molecule include, but are not limited to, hormones, ligands, information transfer substances, synthesized by combinatorial chemistry, pharmaceutically acceptable low molecular weight molecules (e.g., low molecular weight ligands and the like), and the like.

As used herein, the term “contact” refers to direct or indirect placement of a compound physically close to the polypeptide or polynucleotide of the present invention. Polypeptides or polynucleotides may be present in a number of buffers, salts, solutions, and the like. The term “contact” includes placement of a compound in a beaker, a microtiter plate, a cell culture flask, a microarray (e.g., a gene chip) or the like containing a polypeptide encoded by a nucleic acid or a fragment thereof.

As used herein, the term “biological activity” refers to activity possessed by an agent (e.g., a polynucleotide, a protein, etc.) within an organism, including activities exhibiting various functions. For example, when a certain agent is a ligand, the biological activity thereof includes the activity of the ligand to bind to a corresponding receptor. When a certain agent is active STAT5 of the present invention, the biological activity thereof includes at least one of the activities possessed by at least STAT5 (nuclear transfer ability, ability to bind to the STAT5 consensus sequence, and the like). In another embodiment, an example of biological activity is an activity of STAT5 functioning as a transcription factor (e.g., ability to bind to a STAT5 consensus sequence).

As used herein, in a non-limiting example, biological activity may be assayed by a method using assays for determining active STAT5.

Polypeptides used in the present invention may be produced by, for example, cultivating primary culture cells producing the peptides or cell lines thereof, followed by separation or purification of the peptides from culture supernatant. Alternatively, genetic manipulation techniques can be used to incorporate a gene encoding a polypeptide of interest into an appropriate expression vector, transform an expression host with the vector, and collect recombinant polypeptides from the culture supernatant of the transformed cells. The above-described host cell may be any host cells conventionally used in genetic manipulation techniques as long as they can express a polypeptide of interest while keeping the physiological activity of the peptide (e.g., E. coli, yeast, an animal cell, etc.). Polypeptides derived from the thus-obtained cells may have at least one amino acid substitution, addition, and/or deletion or at least one sugar chain substitution, addition, and/or deletion as long as they have substantially the same function as that of naturally-occurring polypeptides.

A given amino acid may be substituted with another amino acid in a protein structure, such as a cationic region or a substrate molecule binding site, without a clear reduction or loss of interactive binding ability. A given biological function of a protein is defined by the interactive ability or other property of the protein. Therefore, a particular amino acid substitution may be performed in an amino acid sequence, or at the DNA code sequence level, to produce a protein which maintains the original property after the substitution. Therefore, various modifications of peptides as disclosed herein and DNA encoding such peptides may be performed without clear losses of biological usefulness.

When the above-described modifications are designed, the hydrophobicity indices of amino acids may be taken into consideration. The hydrophobic amino acid indices play an important role in providing a protein with an interactive biological function, which is generally recognized in the art (Kyte, J. and Doolittle, R. F., J. Mol. Biol. 157(1) :105-132, 1982). The hydrophobic property of an amino acid contributes to the secondary structure of a protein and then regulates interactions between the protein and other molecules (e.g., enzymes, substrates, receptors, DNA, antibodies, antigens, etc.). Each amino acid is given a hydrophobicity index based on the hydrophobicity and charge properties thereof as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamicacid (−3.5); glutamine (−3.5); aspartic acid (−3.5); asparagine (−3.5); lysine (−3.9); and arginine (−4.5).

It is well known that if a given amino acid is substituted with another amino acid having a similar hydrophobicity index, the resultant protein may still have a biological function similar to that of the original protein (e.g., a protein having an equivalent enzymatic activity). For such an amino acid substitution, the hydrophobicity index is preferably within ±2, more preferably within ±1, and even more preferably within ±0.5. It is understood in the art that such an amino acid substitution based on hydrophobicity is efficient.

A hydrophilicity index is also useful for modification of an amino acid sequence of the present invention. As described in U.S. Pat. No. 4,554,101, amino acid residues are given the following hydrophilicity indices: arginine (+3.0); lysine (+3.0); aspartic acid (+3.0±1); glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid may be substituted with another amino acid which has a similar hydrophilicity index and can still provide a biological equivalent. For such an amino acid substitution, the hydrophilicity index is preferably within ±2, more preferably ±1, and even more preferably ±0.5.

The term “conservative substitution” as used herein refers to amino acid substitution in which a substituted amino acid and a substituting amino acid have similar hydrophilicity indices or/and hydrophobicity indices. For example, conservative substitution is carried out between amino acids having a hydrophilicity or hydrophobicity index of within ±2, preferably within ±1, and more preferably within ±0.5. Examples of conservative substitution include, but are not limited to, substitutions within each of the following residue pairs: arginine and lysine; glutamic acid and aspartic acid; serine and threonine; glutamine and asparagine; and valine, leucine, and isoleucine, which are well known to those skilled in the art.

As used herein, the term “variant” refers to a substance, such as a polypeptide, polynucleotide, or the like, which differs partially from the original substance. Examples of such a variant include a substitution variant, an addition variant, a deletion variant, a truncated variant, an allelic variant, and the like. Examples of such a variant include, but are not limited to, a nucleotide or polypeptide having one or several substitutions, additions and/or deletions or a nucleotide or polypeptide having at least one substitution, addition and/or deletion. The term “allele” as used herein refers to a genetic variant located at a locus identical to a corresponding gene, where the two genes are distinguished from each other. Therefore, the term “allelic variant” as used herein refers to a variant which has an allelic relationship with a given gene. Such an allelic variant ordinarily has a sequence the same as or highly similar to that of the corresponding allele, and ordinarily has almost the same biological activity, though it rarely has different biological activity. The term “species homolog” or “homolog” as used herein refers to one that has an amino acid or nucleotide homology with a given gene in a given species (preferably at least 60% homology, more preferably at least 80%, at least 85%, at least 90%, and at least 95% homology). A method for obtaining such a species homolog is clearly understood from the description of the present specification. The term “ortholog” (also called orthologous genes) refers to genes in different species derived from a common ancestry (due to speciation). For example, in the case of the hemoglobin gene family having multigene structure, human and mouse α-hemoglobin genes are orthologs, while the human α-hemoglobin gene and the human β-hemoglobin gene are paralogs (genes arising from gene duplication). Orthologs are useful for estimation of molecular phylogenetic trees. Usually, orthologs in different species may have a function similar to that of the original species. Therefore, orthologs of the present invention may be useful in the present invention.

As used herein, the term “conservative (or conservatively modified) variant” applies to both amino acid and nucleic acid sequences. With respect to particular nucleic acid sequences, conservatively modified variants refer to those nucleic acids which encode identical or essentially identical amino acid sequences. Because of the degeneracy of the genetic code, a large number of functionally identical nucleic acids encode any given protein. For example, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” which represent one species of conservatively modified variation. Every nucleic acid sequence herein which encodes a polypeptide also describes every possible silent variation of the nucleic acid. Those skilled in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence. Preferably, such modification may be performed while avoiding substitution of cysteine which is an amino acid capable of largely affecting the higher-order structure of a polypeptide. Examples of a method for such modification of a base sequence include cleavage using a restriction enzyme or the like; ligation or the like by treatment using DNA polymerase, Klenow fragments, DNA ligase, or the like; and a site specific base substitution method using synthesized oligonucleotides (specific-site directed mutagenesis; Mark Zoller and Michael Smith, Methods in Enzymology, 100, 468-500(1983)). Modification can be performed using methods ordinarily used in the field of molecular biology.

In order to prepare functionally equivalent polypeptides, amino acid additions, deletions, or modifications can be performed in addition to amino acid substitutions. Amino acid substitution(s) refers to the replacement of at least one amino acid of an original peptide chain with different amino acids, such as the replacement of 1 to 10 amino acids, preferably 1 to 5 amino acids, and more preferably 1 to 3 amino acids with different amino acids. Amino acid addition(s) refers to the addition of at least one amino acid to an original peptide chain, such as the addition of 1 to 10 amino acids, preferably 1 to 5 amino acids, and more preferably 1 to 3 amino acids to an original peptide chain. Amino acid deletion(s) refers to the deletion of at least one amino acid, such as the deletion of 1 to 10 amino acids, preferably 1 to 5 amino acids, and more preferably 1 to 3 amino acids. Amino acid modification includes, but is not limited to, amidation, carboxylation, sulfation, halogenation, truncation, lipidation, alkylation, glycosylation, phosphorylation, hydroxylation, acylation (e.g., acetylation), and the like. Amino acids to be substituted or added may be naturally-occurring or nonnaturally-occurring amino acids, or amino acid analogs. Naturally-occurring amino acids are preferable.

As used herein, the term “peptide analog” refers to a compound which is different from a peptide but has at least one chemical or biological function equivalent to the peptide. Therefore, a peptide analog includes one that has at least one amino acid analog or amino acid derivative addition or substitution with respect to the original peptide. A peptide analog has the above-described addition or substitution so that the function thereof is substantially the same as the function of the original peptide (e.g., a similar pKa value, a similar functional group, a similar binding manner to other molecules, a similar water-solubility, and the like). Such a peptide analog can be prepared using a technique well known in the art. Therefore, a peptide analog may be a polymer containing an amino acid analog.

A chemically-modified polypeptide composition in which a polypeptide of the present invention is attached to a polymer is included within the scope of the present invention. This polymer may be water soluble so that the protein does not precipitate in an aqueous environment (e.g., a physiological environment). An appropriate water soluble polymer may be selected from the group consisting of: polyethylene glycol (PEG), monomethoxy-polyethylene glycol, dextran, cellulose, or other carbohydrate based polymers, poly-(N-vinylpyrrolidone) polyethylene glycol, propylene glycol homopolymers, a polypropylene oxide/ethylene oxide co-polymer, polyoxyethylated polyols (e.g., glycerol) and polyvinyl alcohol. The selected polymer is typically modified to have a single reactive group (e.g., active ester for acylation or aldehyde for alkylation). As a result, the degree of polymerization may be controlled. The polymer may be of any molecular weight, and maybe branched or unbranched. Included within the scope of suitable polymers is a mixture of polymers. When the chemically modified polymer of the present invention is used in therapeutic applications, a pharmaceutically acceptable polymer is selected.

When the polymer is modified by an acylation reaction, the polymer should have a single reactive ester group. Alternatively, when the polymer is modified by reducing alkylation, the polymer should have a single reactive aldehyde group. A preferable reactive aldehyde is, for example, polyethylene glycol, propionaldehyde (which is water stable), or mono C1-C10 alkoxy or aryloxy derivatives thereof (see U.S. Pat. No. 5,252,714, which is herein incorporated by reference in its entity).

Pegylation of the polypeptide of the present invention may be carried out by any of the pegylation reactions known in the art, as described for example in the following references: Focus on Growth Factors, 3, 4-10 (1992); EP 0 154 316; EP 0 401 384, which are herein incorporated by reference in their entity). Preferably, pegylation may be carried out via an acylation reaction or an alkylation reaction with a reactive polyethylene glycol molecule (or an analogous reactive water-soluble polymer). Polyethylene glycol (PEG) is a water-soluble polymer suitable for use in pegylation of the polypeptide of the present invention (e.g., STAT5, an active form thereof or an activating agent therefor, and the like). As used herein, the term “polyethylene glycol” is meant to encompass any of the forms of PEG that have been used to derivatize proteins (e.g., mono(C1-C10) alkoxy-polyethylene glycol or mono(C1-C10) aryloxy-polyethylene glycol (PEG)).

Chemical derivatization of the polypeptide of the present invention may be performed under any suitable conditions that can be used to react a biologically active substance with an activated polymer molecule. Methods for preparing pegylated polypeptides of the present invention will generally comprise the steps of (a) reacting the polypeptide with polyethylene glycol (such as a reactive ester or aldehyde derivative of PEG) under conditions whereby STAT5, an active form thereof or an activating agent therefor becomes attached to one or more PEG groups, and (b) obtaining the reaction product(s). The optimal reaction conditions or the acylation reactions are easily selected by those skilled in the art based on known parameters and the desired result.

Generally, conditions may be alleviated or modulated by administration of the pegylated polypeptide of the present invention. However, the polypeptide derivative of the polypeptide molecule of the present invention disclosed herein may have additional activities, enhanced or reduced biological activity, or other characteristics (e.g., increased or decreased half-life), as compared to the nonderivatized molecules. The polypeptide of the present invention, and fragments, variants and derivatives thereof may be used singly or in combination, or in combination with other pharmaceutical compositions.

A substance of the present invention may be in the form of a nucleic acid (nucleic acid molecule) if the expression product of the substance is consistently or transiently active STAT5. Such a nucleic acid molecule includes one in which a part of the sequence of the nucleic acid is deleted or is substituted with other base(s), or an additional nucleic acid sequence is inserted, as long as a polypeptide expressed by the nucleic acid has substantially the same activity as that of naturally-occurring active STAT5, as described above. Alternatively, an additional nucleic acid may be linked to the 5′ terminus and/or 3′ terminus of the nucleic acid. The nucleic acid molecule may include one that is hybridizable to a gene encoding a polypeptide under stringent conditions and encodes a polypeptide having substantially the same function. Such a gene is known in the art and can be used in the present invention.

The above-described nucleic acid can be obtained by a well-known PCR method, i.e., chemical synthesis. This method may be combined with, for example, site-specific mutagenesis, hybridization, or the like.

As used herein, the term “substitution, addition or deletion” for a polypeptide or a polynucleotide refers to the substitution, addition or deletion of an amino acid or its substitute, or a nucleotide or its substitute, with respect to the original polypeptide or polynucleotide, respectively. This is achieved by techniques well known in the art, including a site-specific mutagenesis technique and the like. A polypeptide or a polynucleotide may have any number (>0) of substitutions, additions, or deletions. The number can be as large as a variant having such a number of substitutions, additions or deletions which maintains an intended function (e.g., the information transfer function of hormones and cytokines, etc.). For example, such a number may be one or several, and preferably within 20% or 10% of the full length, or no more than 100, no more than 50, no more than 25, or the like.

Polypeptides used in the present invention may be derived from any organism. Preferably, the organism is a vertebrate (e.g., the class Mammalia, the class Reptilia, the class Amphibia, the class Pisces, the class Aves, and the like, and more preferably a mammal (e.g., the order Rodentia (e.g., mice, rats, etc.), the order Primates (e.g., humans, etc.), etc.). Polypeptides used in the present invention may be synthesized as long as they have the desired effect. Such polypeptides may be synthesized by synthesis techniques well known in the art. For example, synthesis techniques using automatic solid-phase peptide synthesizers are described in, for example, Stewart, J. M. et al. (1984), Solid Phase Peptide Synthesis, Pierce Chemical Co.; Grant, G. A. (1992), Synthetic Peptides: A User's Guide, W. H. Freeman; Bodanszky, M. (1993), Principles of Peptide Synthesis, Springer-Verlag; Bodanszky, M. et al. (1994), The Practice of Peptide Synthesis, Springer-Verlag; Fields, G. B. (1997), Phase Peptide Synthesis, Academic Press; Pennington, M. W. et al. (1994), Peptide Synthesis Protocols, Humana Press; and Fields, G. B. (1997), Solid-Phase Peptide Synthesis, Academic Press.

Polypeptides used in the present invention may be expressed as fusion proteins with only a hinge region of an antibody, so that the resultant polypeptides form a dimer via disulfide bond. Alternatively, polypeptides used in the present invention may be expressed as fusion proteins which have disulfide bonds at the C-terminus, the N-terminus, or other positions while keeping the activity, so that the resultant polypeptides form a multimer which is a dimer or higher order oligomers. Sequences of the present invention, such as a sequence set forth in SEQ ID NO:2 or 4 and the like, may be linked with one another in series to produce a multimeric structure. Therefore, the polypeptides of the present invention in the form of any dimer or higher order oligomers, which are produced by genetic engineering techniques, are within the scope of the present invention. Alternatively, the polypeptides of the present invention in the form of a dimer or higher order oligomers may contain monomers of the same or different species. Therefore, the dimer of the present invention may be a homodimer or a heterodimer.

(Immunology)

As used herein, the term “antibody” is used in its ordinary sense, which is well known to those skilled in the art. As used herein, the term “antibody” encompasses the whole molecule and a fragment thereof, a derivative thereof, a conjugate thereof, and the like. Preferably, antibodies, which are preferably used in the present invention, recognize a polypeptide of the present invention, more preferably in a specific manner. Such antibodies may be polyclonal antibodies or monoclonal antibodies. In one embodiment of the present invention, such antibodies are also within the scope of the present invention.

As used herein, the term “antigen” refers to a substance (e.g., not limited to, a protein, a lipid, a sugar, or the like) which binds to an antibody or a specific receptor of a B lymphocyte, a T lymphocyte, or the like to elicit an immune reaction (e.g., antibody production and/or cytopathy, or the like). The ability to bind to an antibody or a lymphocyte receptor is called “antigenicity”. The ability to induce an immune response, such as antibody production or the like, is called “immunogenicity”. A substance used as an antigen contains at least one material of interest (e.g., a protein, etc.). A substance to be contained with preferably has its full length, or alternatively, may have a partial sequence as long as the sequence contains at least one epitope capable of eliciting immune response. As used herein, the term “epitope” or “antigenic determinant” refers to a site of an antigen molecule to which an antibody or a lymphocyte receptor binds. Techniques for determining epitopes are well known in the art. Epitopes can be determined by those skilled in the art using the well-known routine techniques if the primary sequence of a nucleic acid or amino acid is provided.

Epitopes can be used even if their exact sites and detailed structures are not clarified. Therefore, the term “epitope” includes a set of amino acid residues which are involved in recognition by a particular immunoglobulin, or in the context of T cells, those residues necessary for recognition by T cell receptor proteins and/or Major Histocompatibility Complex (MHC) receptors. This term is also used interchangeably with “antigenic determinant” or “antigenic determinant site”. In the field of immunology, in vivo or in vitro, an epitope is the feature of a molecule (e.g., primary, secondary and tertiary peptide structure, and charge) that forms a site recognized by an immunoglobulin, T cell receptor or HLA molecule. An epitope including a peptide comprises 3 or more amino acids in a spatial conformation which is unique to the epitope. Generally, an epitope consists of at least 5 such amino acids, and more ordinarily, consists of at least 6, 7, 8, 9 or 10 such amino acids. The greater the length of an epitope, the more the similarity of the epitope to the original peptide, i.e., longer epitopes are generally preferable. This is not necessarily the case when the conformation is taken into account. Methods of determining the spatial conformation of amino acids are known in the art, and include, for example, X-ray crystallography and two-dimensional nuclear magnetic resonance spectroscopy. Furthermore, the identification of epitopes in a given protein is readily accomplished using techniques well known in the art. See, also, Geysen et al., Proc. Natl. Acad. Sci. USA (1984) 81: 3998 (general method for rapidly synthesizing peptides to determine the location of immunogenic epitopes in a given antigen); U.S. Pat. No. 4,708,871 (procedures for identifying and chemically synthesizing epitopes of antigens); and Geysen et al., Molecular Immunology (1986) 23: 709 (technique for identifying peptides with high affinity for a given antibody) Antibodies that recognize the same epitope can be identified in a simple immunoassay. Thus, methods for determining an epitope including a peptide are well known in the art. Such an epitope can be determined using a well-known, common technique by those skilled in the art if the primary nucleic acid or amino acid sequence of the epitope is provided.

Therefore, an epitope including a peptide requires a sequence having a length of at least 3 amino acids, preferably at least 4 amino acids, more preferably at least 5 amino acids, at least 6 amino acids, at least 7 amino acids, at least 8 amino acids, at least 9 amino acids, at least 10 amino acids, at least 15 amino acids, at least 20 amino acids, and at least 25 amino acids. Epitopes may be linear or folded.

The structure of polymers (e.g., polypeptide structure) may be described at various levels. This structure is generally described in, for example, Alberts et al., Molecular Biology of the Cell (3rd Ed., 1994), and Cantor and Schimmel, Biophysical Chemistry Part I: The Conformation of Biological Macromolecules (1980). The term “primary structure” refers to an amino acid sequence of a particular peptide. The term “secondary structure” refers to a three-dimensional structure locally provided in a polypeptide. Such a structure is generally known as a domain. A domain forms a small unit of polypeptide, and representatively, are portions having a length of about 50 to 350 amino acids. Representative domains are made of a stretch of β-sheets (β-strands, etc.) and/or α-helices. The term “tertiary structure” refers to the complete structure of a polypeptide monomor. The term “quaternary structure” refers to a three-dimensional structure formed by non-covalent association of separate three-dimensional units. Terms relating to anisotropy are used in the same sense as in the field of energy. Therefore, polypeptides of the present invention may encompass polypeptides having any amino acid sequence as long as their higher order structure has substantially the same ability as that of active STAT5.

As used herein, the term “specifically expressed” in the case of genes indicates that a gene is expressed in a specific site or for a specific period of time at a level different from (preferably higher than) that in other sites or periods of time. The term “specifically expressed” indicates that a gene may be expressed only in a given site (specific site) or may be expressed in other sites. Preferably, the term “specifically expressed” indicates that a gene is expressed only in a given site. Therefore, a gene encoding active STAT5 of the present invention can be manipulated so as to express at a specific site or in a specific period of time. Such a technique is well known in the art and is described in literature referenced herein.

(General Techniques)

Molecular biological techniques, biochemical techniques, and microorganism techniques as used herein are well known in the art and commonly used, and are described in, for example, Sambrook J. et al. (1989), Molecular Cloning: A Laboratory Manual, Cold Spring Harbor and its 3rd Ed. (2001); Ausubel, F. M. (1987), Current Protocols in Molecular Biology, Greene Pub. Associates and Wiley-Interscience; Ausubel, F. M. (1989), Short Protocols in Molecular Biology: A Compendium of Methods from Current Protocols in Molecular Biology, Greene Pub. Associates and Wiley-Interscience; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates; Sninsky, J. J. et al. (1999), PCR Applications: Protocols for Functional Genomics, Academic Press; Special issue, Jikken Igaku [Experimental Medicine] “Idenshi Donyu & Hatsugenkaiseki Jikkenho [Experimental Method for Gene Introduction & Expression Analysis]”, Yodo-sha, 1997; and the like. Relevant portions (or possibly the entirety) of each of these publications are herein incorporated by reference.

DNA synthesis techniques and nucleic acid chemistry for preparing artificially synthesized genes are described in, for example, Gait, M. J. (1985), Oligonucleotide Synthesis: A Practical Approach, IRL Press; Gait, M. J. (1990) Oligonucleotide Synthesis: A Practical Approach, IRL Press; Eckstein, F. (1991), Oligonucleotides and Analogues: A Practical Approach, IRL Press; Adams, R. L. et al. (1992), The Biochemistry of the Nucleic Acids, Chapman & Hall; Shabarova, Z. et al. (1994), Advanced Organic Chemistry of Nucleic Acids, Weinheim; Blackburn, G. M. et al. (1996), Nucleic Acids in Chemistry and Biology, Oxford University Press; Hermanson, G. T. (1996), Bioconjugate Techniques, Academic Press; and the like, related portions of which are herein incorporated by reference.

(Genetic Engineering)

STAT5 and the like, and fragments and variants thereof as used herein can be produced by genetic engineering techniques.

When a gene is mentioned herein, the term “vector” or “recombinant vector” refers to a vector capable of transferring a polynucleotide sequence of interest to a target cell. Such a vector is capable of self-replication or incorporation into a chromosome in a host cell (e.g., a prokaryotic cell, yeast, an animal cell, a plant cell, an insect cell, an individual animal, and an individual plant, etc.), and contains a promoter at a site suitable for transcription of a polynucleotide of the present invention. As used herein, a vector may be a plasmid.

As used herein, the term “viral vector” refers to a vector derived from a virus. As used herein, the term “virus” refers to an infectious small construct having DNA or RNA as a genome and being capable of proliferating only within infected cells. Viruses are selected from the group consisting of Retroviridae, Togaviridae, Coronaviridae, Flaviviridae, Paramyxoviridae, Orthomyxoviridae, Bunyaviridae, Rhabdoviridae, Poxviridae, Herpesviridae, Baculoviridae, and Hepadnaviridae. As used herein, the term “retrovirus” refers to a virus which has genetic information in the form of RNA and synthesizes DNA using reverse transcriptase based on the RNA information.

As used herein, the term “retrovirus vector” refers to a retrovirus which is used as a carrier (vector) carrying a gene. Examples of a retrovirus vector used in the present invention include, but are not limited to, a retrovirus type expression vector based on Moloney Murine Leukemia Virus (MMLV), Murine Stem Cell Virus (MSCV), or the like. Preferably, in a non-limiting example, a retrovirus vector includes pGen-, pMSCV, and the like.

As used herein, the term “expression vector” refers to a nucleic acid sequence comprising a structural gene and a promoter for regulating expression thereof, and in addition, various regulatory elements in a state that allows them to operate within host cells. The regulatory element may include, preferably, terminators, selectable markers such as drug-resistance genes, and enhancers. In the case of humans, an expression vector used in the present invention may further include pCAGGS (Niwa H., et al, Gene; 108: 193-9(1991)).

As used herein, the term “recombinant vector” refers to a vector capable of transferring a polynucleotide sequence of interest to a target cell. Such a vector is capable of self-replication or incorporation into a chromosome in a host cell, and contains a promoter at a site suitable for transcription of a polynucleotide of the present invention.

As used herein, a “recombinant vector” for animal cells includes pcDNA I/Amp, pcDNA I, pCDM8 (all commercially available from Funakoshi), pAGE107 (Japanese Laid-Open Publication No. 3-22979; Cytotechnology, 3, 133(1990)), pREP4 (Invitrogen), pAGE103 (J. Biochem., 101, 1307(1987)) pAMo, pAMoA (J. Biol. Chem., 268, 22782-22787(1993)), pCAGGS (Niwa H., et al, Gene; 108: 193-9(1991)), and the like.

As used herein, the term “terminator” refers to a sequence which is located downstream of a protein-encoding region of a gene and which is involved in the termination of transcription when DNA is transcribed into mRNA, and the addition of a poly A sequence. It is known that a terminator contributes to the stability of mRNA, and has an influence on the amount of gene expression. Examples of a terminator include, but are not limited to, terminators derived from mammals, the CaMV35S terminator, the terminator of the nopaline synthase gene (Tnos), the terminator of the tobacco PR1a gene, and the like.

As used herein, the term “promoter” refers to a base sequence which determines the initiation site of transcription of a gene and is a DNA region which directly regulates the frequency of transcription. Transcription is started by RNA polymerase binding to a promoter. A promoter region is usually located within about 2 kbp upstream of the first exon of a putative protein coding region. Therefore, it is possible to estimate a promoter region by predicting a protein coding region in a genomic base sequence using DNA analysis software. A putative promoter region is usually located upstream of a structural gene, but depending on the structural gene, i.e., a putative promoter region may be located downstream of a structural gene. Preferably, a putative promoter region is located within about 2 kbp upstream of the translation initiation site of the first exon.

As used herein, the term “site-specific” in relation to gene expression generally refers to specificity of the gene expression at a site (e.g., the heart, myocardial cells, or the like in animals; etc.) of an organism (e.g., an animal). The term “time-specific” refers to specificity of gene expression with respect to a particular stage (e.g., upon stroke, or the like) of an organism (e.g., an animal). Such specificity can be introduced into a desired organism using an appropriately selected promoter.

As used herein, the term “structural” in relation to expression of a promoter of the present invention indicates that the promoter is expressed at substantially a constant level no matter whether organisms are in the juvenile phase or mature phase of the growth thereof. Specifically, when northern blotting is performed under the same conditions as those described in the examples below, expression is defined as being structural if the expression is substantially observed at any time point (e.g., at the same two sites or more, or corresponding sites). Structural promoters are considered to play a role in homeostasis of organisms in ordinary growth environments. The term “stress responsive” in relation to expression of a promoter of the present invention indicates that the level of expression of the promoter is changed when at least one stress (e.g., differentiating stimuli, or the like) is given to organisms. In particular, the term “stress inductive” indicates that the expression level is increased, and the term “stress reductive” indicates that the expression level is decreased. “Stress reductive” expression is based on the premise that expression is normally observed, overlapping the concept of “structural” expression. These properties can be determined by extracting RNA from any portion of an organism and analyzing the level of expression by northern blotting or RT-PCR, or quantitating expressed proteins by western blotting. When a vector having an incorporated nucleic acid encoding a polypeptide used in the present invention with a stress inductive promoter is used in order to transform an animal or a part thereof (a specific cell, tissue, or the like), the peptide can be expressed under given conditions (e.g., stimulation for differentiation) using a stimulating agent having activity to induce the promoter.

As used herein, the term “enhancer” refers to a sequence which is used so as to enhance the expression efficiency of a gene of interest. When used in plants, an enhancer region containing a sequence upstream of a human cytomegalovirus immediate-early enhancer is preferable. One or more enhancers may be used, or no enhancer may be used.

As used herein, the term “operatively linked” indicates that a desired sequence is located such that expression (operation) thereof is under control of a transcription and translation regulatory sequence (e.g., a promoter, an enhancer, and the like) or a translation regulatory sequence. In order for a promoter to be operatively linked to a gene, typically, the promoter is located immediately upstream of the gene. A promoter is not necessarily adjacent to a structural gene.

The present invention may be used in any animal. Techniques for use of the present invention in animals are well known in the art and are commonly used, as described in, for example, Ausubel F. A. et al., editors, (1988), Current Protocols in Molecular Biology, Wiley, New York, N.Y.; Sambrook J. et al. (1987) Molecular Cloning: A Laboratory Manual, 2nd Ed. and its 3rd Ed., Cold Spring Harbor Laboratory Press, Cold Spring Harbor, N.Y.; Special issue, Jikken Igaku [Experimental Medicine] “Experimental Method for Gene Introduction & Expression Analysis”, Yodo-sha, 1997; and the like.

As used herein, the term “transformant” refers to the whole or a part of an organism, such as a cell, which is produced by transformation. Examples of a transformant include animal cells and the like. Transformants may be referred to as transformed cells, transformed tissue, transformed hosts, or the like, depending on the subject. As used herein, all of the forms are encompassed, however, a particular form may be specified in a particular context.

The term “animal” is used herein in its broadest sense and refers to vertebrates and invertebrates (e.g., arthropods). Examples of animals include, but are not limited to, any of the class Mammalia, the class Aves, the class Reptilia, the class Amphibia, the class Pisces, the class Insecta, the class Vermes, and the like. Preferably, the animal may be a mammal.

It will be understood that polypeptides, nucleic acids, kits, systems, compositions, and methods for use in the present invention can function in all animals including mammals and other species. This is because ligands corresponding to STAT5 are known to be present in animals in addition to mammals.

The term “cell” is herein used in its broadest sense in the art, referring to a structural unit of tissue of a multicellular organism, which is capable of self replicating, has genetic information and a mechanism for expressing it, and is surrounded by a membrane structure which isolates the living body from the outside.

As used herein, the term “tissue” in relation to organisms refers to an aggregate of cells having substantially the same function. Therefore, a tissue may be a part of an organ. Organs usually have cells having the same function, and may have coexisting cells having slightly different functions. Therefore, as used herein, tissues may have various kinds of cells as long as a certain property is shared by the cells.

As used herein, the term “organ” refers to a structure which has a single independent form and in which one or more tissues are associated together to perform a specific function. In animals, examples of organs include, but are not limited to, stomach, liver, intestine, pancreas, lung, airway, nose, heart, artery, vein, lymph node (lymphatic system), thymus, ovary, eye, ear, tongue, skin, and the like.

As used herein, the term “treat by active STAT5, or STAT5 and an agent capable of activating the STAT5” indicates that cells are exposed to the active STAT5 of the present invention, or STAT5 and the agent capable of activating the STAT5, so as to expand the cells. Therefore, such a treatment includes techniques, such as: directly contacting cells with active STAT5; transforming cells so that the agent is transiently expressed therein; or the like.

As used herein, the term “transgenic” refers to incorporation of a specific gene into an organism (e.g., animals (mice, etc.) or plants) or such an organism having an incorporated gene.

When transgenic organisms are used in the present invention, the transgenic organisms can be produced by a microinjection method (a trace amount injection method), a viral vector method, an embryonic stem (ES) cell method, a sperm vector method, a chromosome fragment introducing method (transsomic method), an episome method, or the like. These transgenic animal producing techniques are well known in the art.

(Screening)

In the present invention, it is intended to provide a drug by computer modeling based on the disclosure of the present invention.

As used herein, the term “screening” refers to selection of a target, such as an organism, a substance, or the like, a given specific property of interest from a population containing a number of elements using a specific operation/evaluation method. For screening, an agent (e.g., an antibody), a polypeptide or a nucleic acid molecule of the present invention can be used. Screening may be performed using libraries obtained in vitro, in vivo, or the like (with a system using a real substance) or alternatively in silico (with a system using a computer). It will be understood that the present invention encompasses compounds having desired activity obtained by screening. The present invention is also intended to provide drugs which are produced by computer modeling based on the disclosures of the present invention.

In one embodiment, the present invention provides an assay for screening candidate compounds or test compounds for a protein or polypeptide of the present invention, or a compound capable of binding to a biologically active portion thereof or modulating the activity thereof. The test compounds of the present invention can be obtained using any of the numerous approaches in combinatorial library methods known in the art, including biological libraries, spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, nonpeptide oligomer, or small molecule libraries of compounds (Lam (1997) Anticancer Drug Des. 12: 145).

Examples of methods for the synthesis of molecular libraries can be found in the art as follows: DeWitt et al. (1993) Proc. Natl. Acad. Sci. USA 90: 6909; Erb et al. (1994) Proc. Natl. Acac. Sci. USA 91: 1 11422; Zuckermann et al. (1994) J. Med. Chem. 37: 2678; Cho et al. (1993) Science 261: 1303; Carrell et al. (1994) Angew. Chem. Int. Ed. Engl. 33: 2059; Carrell et al. (1994) Angew. Chem. Int. Ed. Engl. 33: 2061; and Gallop et al. (1994) J. Med Chem. 37: 1233.

Libraries of compounds may be presented in solution (e. g., Houghten (1992) Bio Techniques 13: 412-421), or on beads (Lam (1-991) Nature 354: 82-84), chips (Fodor (1993) Nature 364: 555-556), bacteria (Ladner, U.S. Pat. No. 5,223,409), spores (Landner, supra), plasmids (Cull et al. (1992) Proc. Natl. Acad. Sci. USA 89: 1865-1869), or phages (Scott and Smith (1990). Science 249: 386-390; Devlin (1990) Science 249. 404-406; Cwirla et al. (1990) Proc. Natl. Acad. Sci. USA 87: 6378-6382, and Felici (1991) J. Mol. Biol. 222: 301-310; Ladner supra).

In another embodiment of the present invention, a compound is also provided, which is used as a tool for screening for an agent effective as an active component (e.g., a polypeptide or a nucleic acid) of the present invention and which is obtained by a quantitative structure activity relationship (QSAR) modeling technique using a computer. Here, the computer technique includes several substrate templates prepared by a computer, pharmacophores, homology models of an active portion of the present invention, and the like. In general, a method for modeling a typical characteristic group of a substance, which interacts with another substance, based on data obtained in vitro includes a recent CATALYST™ pharmacophore method (Ekins et al., Pharmacogenetics, 9:477 to 489, 1999; Ekins et al., J. Pharmacol. & Exp. Ther., 288:21 to 29, 1999; Ekins et al., J. Pharmacol. & Exp. Ther., 290:429 to 438, 1999; Ekins et al., J. Pharmacol. & Exp. Ther., 291:424 to 433, 1999), a comparative molecular field analysis (CoMFA) (Jones et al., Drug Metabolism & Disposition, 24:1 to 6, 1996), and the like. In the present invention, computer modeling may be performed using molecule modeling software (e.g., CATALYST™ Version 4 (Molecular Simulations, Inc., San Diego, Calif.), etc.).

The fitting of a compound with respect to an active site can be performed using any of various computer modeling techniques known in the art. Visual inspection and manual operation of a compound with respect to an active site can be performed using a program, such as QUANTA (Molecular Simulations, Burlington, Mass., 1992), SYBYL (Molecular Modeling Software, Tripos Associates, Inc., St. Louis, Mo., 1992), AMBER (Weiner et al., J. Am. Chem. Soc., 106:765-784, 1984), CHARMM (Brooks et al., J. Comp. Chem., 4:187 to 217, 1983), or the like. In addition, energy minimization can be performed using a standard force field, such as CHARMM, AMBER, or the like. Examples of other specialized computer modeling methods include GRID (Goodford et al., J. Med. Chem., 28:849 to 857, 1985), MCSS (Miranker and Karplus, Function and Genetics, 11:29 to 34, 1991), AUTODOCK (Goodsell and Olsen, Proteins: Structure, Function and Genetics, 8:195 to 202, 1990), DOCK (Kuntz et al., J. Mol. Biol., 161:269 to 288, 1982), and the like. Further, structural compounds can be newly constructed using an empty active site, an active site of a known small molecule compound with a computer program, such as LUDI (Bohm, J. Comp. Aid. Molec. Design, 6:61 to 78, 1992), LEGEND (Nishibata and Itai, Tetrahedron, 47:8985, 1991), LeapFrog (Tripos Associates, St. Louis, Mo.), or the like. The above-described modeling methods are commonly used in the art. Compounds (e.g., equivalents of active STAT5) encompassed by the present invention can be appropriately designed by those skilled in the art based on the disclosure of the present specification.

(Administration and Injection of Medicament)

Cells prepared using an agent of the present invention (e.g., stem cells, cells differentiated therefrom (e.g., myocardinal cells)) or cell compositions may be in any dosage form as long as the dosage form is suited for transfer into organisms. Examples of the dosage form include, but are not limited to, liquid drug, injection drug, sustained-release drug, and the like. Examples of administration route include, but are not limited to, oral adiministration, parenteral administration, direct administration to affected portions, and the like.

Injection drugs can be prepared using techniques well known in the art. For example, an agent of the present invention is dissolved in an appropriate solvent (physiological saline, buffer (e.g., PBS, etc.), sterilized water, etc.), followed by filter sterilization using a filter, or the like. Thereafter, the solution is placed into a sterile container (e.g., an ampoule, or the like). Thus, an injection drug can be prepared. The injection drug may contain a commonly used pharmaceutical carrier if required. An administration method using a non-invasive catheter may be employed.

In one embodiment, an agent of the present invention (e.g., active STAT5 or a nucleic acid encoding the same, or the like) may be provided in a sustained-release form. Any sustained-released dosage form may be used in the present invention. Examples of sustained-release dosage forms include, but are not limited to, rod-like formulations (e.g., pellet-like, cylinder-like, needle-like formulations, etc.), tablet formulations, disk-like formulations, sphere-like formulations, sheet-like formulations, and the like. Methods for preparing sustained-release dosage forms are well known in the art, as described in, for example, the Japanese Pharmacopeia, the U.S. Pharmacopeia, Pharmacopeias of other countries, and the like. Examples of a method for producing sustained-release drugs include, but are not limited to, a method using disaggregation of a drug from a complex, a method for preparing an aqueous suspened liquid drug, a method for preparing an oil injection liquid or oil suspended injection liquid, a method for preparing an emulsified injection liquid (o/w or w/o type emulsified injection liquid, or the like), and the like.

A composition or kit of the present invention may further comprise a biocompatible material. The biocompatible material may contain at least one selected from the group consisting of silicone, collagen, gelatin, a copolymer of glycolic acid and lactic acid, an ethylene-vinyl acetate copolymer, polyurethane, polyethylene, polytetrafluoroethylene, polypropylene, polyacrylate, and polymethacrylate. Silicone is preferable since it is easy to mold. Examples of biodegradable polymers include, but are not limited to, polymers and copolymers synthesized by non-catalytic dehydrocondensation from at least one selected from the group consisting of collagen, gelatin, α-hydroxycarboxylic acids (e.g., glycolic acid, lactic acid, hydroxybutyric acid, etc.), hydroxydicarboxylic acids (e.g., malic acid, etc.), and hydroxytricarboxylic acid (e.g., citric acid, etc.) or a mixture thereof; poly-acid anhydride, such as poly-α-cyanoacrylic ester, polyamino acid (e.g., poly-γ-benzyl-L-glutamic acid, etc.), maleic anhydride-based copolymers (e.g., styrene-maleic acid copolymers, etc.), and the like; and the like. The pattern of condensation may be any of random, block, and graft. If α-hydroxycarboxylic acids, hydroxydicarboxylic acids, and hydroxytricarboxylic acids have a center of optical activity, any of the D-isomers, L-isomers, and DL-isomers can be used. Preferably, glycolic acid-lactic acid copolymers may be used.

When a composition of the present invention comprising a nucleic acid molecule is administered, the nucleic acid molecule may be administered in the form of a non-viral vector or a viral vector, or naked DNA (direct administration). The administration forms are well known in the art as described in, for example, Special issue, Jikken Igaku [Experimental Medicine] “Idenshi Chiryo no Kisogijyutsu [Basic Techniques for Gene Therapy]”, Yodo-sha, 1996; Special issue, Jikken Igaku [Experimental Medicine] “Idenshi Donyu & Hatsugenkaiseki Jikkenho [Experimental Method for Gene Introduction & Expression Analysis]”, Yodo-sha, 1997; and the like.

In a particular embodiment of the present invention, the nucleic acid sequence of a normal gene or a nucleic acid comprising a sequence encoding an antibody or a functional derivative thereof is administered for the purpose of gene therapy for treatment, inhibition, or prophylaxis of a disease or a disorder associated with abnormal expression and/or activity of a polypeptide of the present invention. Gene therapy means that subjects are treated by administering an expressed or expressible nucleic acid thereto. In this embodiment of the present invention, a protein encoded by a nucleic acid is produced and the protein mediates a therapeutic effect.

Any technique available in the art for gene therapy may be employed in the present invention. Illustrative techniques are described as follows.

Gene therapy techniques are generally reviewed in, for example, Goldspiel et al., Clinical Pharmacy 12: 488-505(1993); Wu and Wu, Biotherapy 3: 87-95(1991); Tolstoshev, Ann. Rev. Pharmacol. Toxicol., 32: 573-596(1993); Mulligan, Science 260: 926-932(1993); and Morgan and Anderson, Ann. Rev. Biochem., 62: 191-217(1993); May, TIBTECH 11(5): 155-215(1993). Recombinant DNA techniques generally known, which are generally used in gene therapy, are described in, for example, Ausubel et al. (ed.), Current Protocols in Molecular Biology, John Wiley & Sons, NY (1993); and Kriegler, Gene Transfer and Expression, A Laboratory Manual, Stockton Press, NY (1990).

Therefore, in the present invention, gene therapy using a nucleic acid molecule encoding STAT5 or a variant or fragment thereof may be useful.

Instead of viral vectors, a gene may be introduced into organisms as follows: a method of introducing a nucleic acid molecule using a liposome (e.g., a liposome method, an HVJ-liposome method, a cationic liposome method, a lipofectin method, a lipofectamin method, etc.); a microinjection method; a gene gun method of transferring a nucleic acid molecule together with a carrier (e.g., gold particle, etc.); and the like. Examples of an expression vector include, but are not limited to, pCAGGS (Gene 108: 193-9, Niwa H., Yamamura K., Miyazaki J. (1991)), pBJ-CMV, pcDNA 3.1, pZeoSV (available from Invitrogen, Stratagene, etc.), and the like.

The HVJ-liposome method comprises including a nucleic acid molecule in a liposome made of lipid double membrane and fusing the liposome with inactivated Sendai virus (Hemagglutinating virus of Japan, HVJ). The HVJ-liposome method has a higher level of activity to fuse with cell membrane than conventional liposome methods. A method for preparing HVJ-liposomes is described in detail in, for example, Special issue, Jikken Igaku [Experimental Medicine] “Idenshi Chiryo no Kisogijyutsu [Basic Techniques for Gene Therapy]”, Yodo-sha, 1996; Special issue, Jikken Igaku [Experimental Medicine] “Idenshi Donyu & Hatsugenkaiseki Jikkenho [Experimental Method for Gene Introduction & Expression Analysis]”, Yodo-sha, 1997; and the like. As HVJ, any strain thereof can be available (e.g., ATCC VR-907, ATCC VR-105, etc.), preferably Z strain.

When a composition of the present invention is provided in the form of a nucleic acid of a viral vector, viral vectors, such as recombinant adenoviruses, retroviruses, and the like, are available. A gene can be introduced into cells or tissues as follows: a nucleic acid encoding active STAT5 or a nucleic acid encoding STAT5 is introduced into a DNA or RNA virus (e.g., detoxified retrovirus, adenovirus, adeno-associated virus, herpes virus, vaccinia virus, poxvirus, poliomyelitis virus, Sindbis virus, Sendai virus, SV40, human immunodeficiency virus (HIV), or the like; and cells or tissues are infected with the recombinant virus. Among these viral vectors, adenovirus has a dramatically higher level of infection efficiency than that of other viral vectors. Adenovirus vectors are preferable.

In the case of the naked DNA method, an expression plasmid (a non-viral vector as described) is dissolved in physiological saline or the like and the resultant solution is administered as it is. Such a solution can be directly injected into a tissue of an organ of an organism by a method described in, for example, Tsurumi Y., Kearney M., Chen D., Silver M., Takeshita S., Yang J., Symes J. F., Isner J. M., Circulation 98 (Suppl. II), 382-388(1997).

Therefore, the amount of a polypeptide (e.g., active STAT5, etc.) contained as an active component in a composition and kit of the present invention may be, for example, about 1 μg to about 1000 mg for an adult (weight: about 60 kg), preferably about 5 μg to about 100 mg. The lower limit of the amount range of the polypeptide is any value between about 1 μg and about 1 mg, such as, for example, about 1 μg, about 2 μg, about 3 μg, about 4 μg, about 5 μg, about 6 μg, about 7 μg, about 8 μg, about 9 μg, about 10 μg, about 15 μg, about 20 μg, etc. The upper limit of the amount range of the polypeptide is any value between about 1000 mg and about 1 mg, such as, about 1000 mg, about 900 mg, about 800 mg, about 700 mg, about 600 mg, about 500 mg, about 400 mg, about 300 mg, about 200 mg, about 100 mg, about 75 mg, about 50 mg, about 25 mg, about 10 mg, about 5 mg, etc.

When an active component of the present invention is in the form of a nucleic acid (e.g., a nucleic acid encoding active STAT5 or a nucleic acid encoding STAT5, etc.), the amount of the nucleic acid may be about 1 μg to about 10 mg for an adult (weight: about 60 kg), preferably about 1 μg to about 1000 μg, and more preferably about 5 μg to about 400 μg. The lower limit of the amount range of the nucleic acid may be any value between about 1 μg and about 20 μg, such as, for example, about 1 μg, about 2 μg, about 3 μg, about 4 μg, about 5 μg, about 6 μg, about 7 μg, about 8 μg, about 9 μg, about 10 μg, about 15 μg, about 20 μg, etc. The upper limit of the amount range of the nucleic acid may be any value between about 10 mg and about 10 μg, such as, for example, about 10 mg, about 9 mg, about 8 mg, about 7 mg, about 6 mg, about 5 mg, about 4 mg, about 3 mg, about 2 mg, about 1 mg, about 750 μg, about 500 μg, about 250 μg, about 100 μg, etc. When two or more cellular physiologically active substances (e.g., SCF, etc.) are contained, the above-described amounts are also applicable. When a viral vector or a non-viral vector is administered, the amount is usually 0.0001 to 100 mg, preferably 0.001 to 10 mg, and more preferably 0.01 to 1 mg. The frequency of administration includes, for example, daily to once per several months (e.g., once per week to once per month).

The composition of the present invention may contain cells prepared in the present invention in amounts, for example, about 1×10³ cells to about 1×10¹¹ cells, preferably about 1×10⁴ cells to about 1×10¹⁰ cells, more preferably about 1×10⁵ cells to about 1×10⁹ cells, and the like. These cells may be present as a solution, such as about 0.1 ml, 0.2 ml, 0.5 ml, or 1 ml of physiological saline, or the like. The upper limit of the amount range of cells may be, for example, about 1×10¹¹ cells, about 5×10¹⁰ cells, about 2×10¹⁰ cells, about 1×10¹⁰ cells, about 5×10⁹ cells, about 2×10⁹ cells, about 1×10⁹ cells, about 5×10⁸ cells, about 2×10⁸ cells, about 1×10⁸ cells, about 5×10⁷ cells, about 2×10⁷ cells, about 1×10⁷ cells, and the like. The lower limit of the amount of cells may be, for example, about 1×10³ cells, about 2×10³ cells, about 5×10³ cells, about 1×10⁴ cells, about 2×10⁴ cells, about 5×10⁴ cells, about 1×10⁵ cells, about 2×10⁵ cells, about 5×10⁵ cells, about 1×10⁶ cells, and the like.

As used herein, polypeptide expression may be “detected” or “quantified” by an appropriate method, including mRNA measurement and immunological measurement methods. Examples of the molecular biological measurement methods include a Northern blotting method, a dot blotting method, a PCR method, and the like. Examples of the immunological measurement method include an ELISA method, an RIA method, a fluorescent antibody method, a Western blotting method, an immunohistological staining method, and the like, where a microtiter plate may be used. Examples of a quantification method include an ELISA method, an RIA method, and the like.

As used herein, the term“amount of expression” refers to the amount of a polypeptide or mRNA expressed in a cell of interest. The amount of expression includes the amount of expression at the protein level of a polypeptide of the present invention evaluated by any appropriate method using an antibody of the present invention, including immunological measurement methods (e.g., an ELISA method, a RIA method, a fluorescent antibody method, a Western blotting method, an immunohistological staining method, and the like, or the amount of expression at the mRNA level of a polypeptide of the present invention evaluated by any appropriate method, including molecular biological measurement methods (e.g., a Northern blotting method, a dot blotting method, a PCR method, and the like). The term “change in the amount of expression” indicates that an increase or decrease in the amount of expression at the protein or mRNA level of a polypeptide of the present invention evaluated by an appropriate method including the above-described immunological measurement method or molecular biological measurement method.

As used herein, “instructions” describe a method of administering a medicament of the present invention, or the like for persons who administer, or are administered, the medicament or the like (e.g., physicians, patients, and the like). For example, the instructions may state that a medicament of the present invention is administered immediately after the attack of myocardial infarction (e.g., within 48 hours, within 36 hours, within 24 hours, within 12 hours, within 6 hours, etc.). The instructions may also state that the administration site is skeletal muscle (e.g., via injection, etc.). The instructions are prepared in accordance with a format defined by an authority of a country in which the present invention is practiced (e.g., Health, Labor and Welfare Ministry in Japan, Food and Drug Administration (FDA) in U.S., and the like), explicitly describing that the instructions are approved by the authority. The instructions are so-called package insert and are typically provided in paper media. The instructions are not so limited and may be provided in the form of electronic media (e.g., web sites and electronic mail provided on the Internet).

The present invention targets any “diseases” requiring a volume of stem cells, or cells, tissues, and organs differentiated therefrom. The present invention may be intended to treat diseases or disorders related to differentiated cells, tissues, or organs which can be developed as a result of the differentiation of a stem cell of the present invention.

In one embodiment, the above-described differentiated cells, tissues, or organs may be of the circulatory system (blood cells, etc.). Examples of the diseases or disorders include, but are not limited to, anemia (e.g., aplastic anemia, (particularly, severe aplastic anemia), renal anemia, cancerous anemia, secondary anemia, refractory anemia, etc.), cancer or tumors (e.g., leukemia) and after chemotherapy therefor, hematopoietic-failure, thrombocytopenia, acute myelocytic leukemia (particularly, a first remission (high-risk group), a second remission and thereafter), acute lymphocytic leukemia (particularly, a first remission, a second remission and thereafter), chronic myelocytic leukemia (particularly, chronic period, transmigration period), malignant lymphoma (particularly, a first remission (high-risk group), a second remission and thereafter), multiple myeloma (particularly, an early period after the onset), and the like.

In another embodiment, the above-described differentiated cells, tissues, or organs maybe of the nervous system. Examples of such diseases or disorders include, but are not limited to, dementia, cerebral stroke and sequela thereof, cerebral tumor, spinal injury, and the like.

In another embodiment, the above-described differentiated cells, tissues, or organs may be of the immune system. Examples of such diseases or disorders include, but are not limited to, T-cell deficiency syndrome, leukemia, and the like.

In another embodiment, the above-described differentiated cells, tissues, or organs may be of the motor organ and the skeletal system. Examples of such diseases or disorders include, but are not limited to, fracture, osteoporosis, luxation of joints, subluxation, sprain, ligament injury, osteoarthritis, osteosarcoma, Ewing's sarcoma, osteogenesis imperfecta, osteochondrodysplasia, and the like.

In another embodiment, the above-described differentiated cells, tissues, or organs may be of the skin system. Examples of such diseases or disorders include, but are not limited to, atrichia, melanoma, cutis matignant lympoma, hemangiosarcoma, histiocytosis, hydroa, pustulosis, dermatitis, eczema, and the like.

In another embodiment, the above-described differentiated cells, tissues, or organs may be of the endocrine system. Examples of such diseases or disorders include, but are not limited to, hypothalamus/hypophysis diseases, thyroid gland diseases, accessory thyroid gland (parathyroid) diseases, adrenal cortex/medulla diseases, saccharometabolism abnormality, lipid metabolism abnormality, protein metabolism abnormality, nucleic acid metabolism abnormality, inborn error of metabolism (phenylketonuria, galactosemia, homocystinuria, maple syrup urine disease), analbuminemia, lack of ascorbic acid systhetic ability, hyperbilirubinemia, hyperbilirubinuria, kallikrein deficiency, mast cell deficiency, diabetes insipidus, vasopressin secretion abnormality, dwarfism, Wolman's disease (acid lipase deficiency)), mucopolysaccharidosis VI, and the like.

In another embodiment, the above-described differentiated cells, tissues, or organs may be of the respiratory system. Examples of such diseases or disorders include, but are not limited to, pulmonary diseases (e.g., pneumonia, lung cancer, etc.), bronchial diseases, and the like.

In another embodiment, the above-described differentiated cells, tissues, or organs may be of the digestive system. Examples of such diseases or disorders include, but are not limited to, esophagial diseases (e.g., esophagial cancer, etc.), stomach/duodenum diseases (e.g., stomach cancer, duodenum cancer, etc.), small intestine diseases/large intestine diseases (e.g., polyps of the colon, colon cancer, rectal cancer, etc.), bile duct diseases, liver diseases (e.g., liver cirrhosis, hepatitis (A, B, C, D, E, etc.), fulminant hepatitis, chronic hepatitis, primary liver cancer, alcoholic liver disorders, drug induced liver disorders, etc.), pancreatic diseases (acute pancreatitis, chronic pancreatitis, pancreas cancer, cystic pancreas diseases, etc.), peritoneum/abdominal wall/diaphragm diseases (hernia, etc.), Hirschsprung's disease, and the like.

In another embodiment, the above-described differentiated cells, tissues, or organs may be of the urinary system. Examples of such diseases or disorders include, but are not limited to, kidney diseases (e.g., renal failure, primary glomerulus diseases, renovascular disorders, tubular function abnormality, interstitial kidney diseases, kidney disorders due to systemic diseases, kidney cancer, etc.), bladder diseases (e.g., cystitis, bladder cancer, etc.), and the like.

In another embodiment, the above-described differentiated cells, tissues, or organs may be of the genital system. Examples of such diseases or disorders include, but are not limited to, male genital organ diseases (e.g., male sterility, prostatomegaly, prostate cancer, testicular cancer, etc.), female genital organ diseases (e.g., female sterility, ovary function disorders, hysteromyoma, adenomyosis uteri, uterine cancer, endometriosis, ovarian cancer, villosity diseases, etc.), and the like.

In another embodiment, the above-described differentiated cells, tissues, or organs may be of the circulatory system. Examples of such diseases or disorders include, but are not limited to, heart failure, angina pectoris, myocardial infarct, arrhythmia, valvulitis, cardiac muscle/pericardium diseases, congenital heart diseases (e.g., atrial septal defect, arterial canal patency, tetralogy of Fallot, etc.), artery diseases (e.g., arteriosclerosis, aneurysm), vein diseases (e.g., phlebeurysm, etc.), lymphoduct diseases (e.g., lymphedema, etc.), and the like.

With the stem cell of the present invention, the above-described diseases could be treated while avoiding conventional side effects of transplantation therapy of naturally-occurring stem cells or differentiation cells (particularly, caused by foreign matter or heterogenous cells, (e.g., infection, graft-versus-host diseases, etc.)). This effect is efficiently achieved only after a method is provided which can maintain the pluripotency and self-replication of a stem cell. The effect cannot be conventionally achieved or is difficlut.

(Cell Differentiation and Cell Expansion)

The present invention targets cell which are intended to be expanded. Typically, stem cells maybe used. However, any cells can be employed as long as they can be changed to a desired cell by treatment using an agent of the present invention.

As used herein, the term “stem cell” refers to a cell having self-replication ability and pluripotency, and in actual situations, a cell capable of regenerating a tissue, which has been injured, to some extent. A stem cell for use in the present invention may be an embryonic stem (ES) cell or tissue stem cell (also called tissue specific stem cell or somatic stem cell), Embryonic stem cells are pluripotent stem cells derived from early embryos. An embryonic stem cell was first established in 1981, which has been applied to production of knockout mice since 1989. In 1998, a human embryonic stem cell was established, which is currently becoming available for regenerative medicine. Therefore, in one preferred embodiment of the present invention, embryonic stem cells (embryonic stem cells and embryonic germ cells) may be employed. In another preferred embodiment, tissue stem cells (e.g., bone marrow cells (e.g., hematopoietic stem cells)) may be employed.

Tissue stem cells have a relatively limited level of differentiation unlike embryonic stem cells. Tissue stem cells are present in tissues and have an undifferentiated intracellular structure. Tissue stem cells have a higher nucleus/cytoplasm ratio and have few intracellular organelles. Most tissue stem cells have pluripotency, a long cell cycle, and proliferative ability beyond the life of the individual. Therefore, in a preferred embodiment of the present invention, a tissue stem cell commited to blood cells may be employed.

Tissue stem cells maybe divided into three categories in accordance with their origins: ectoderm, mesoderm, and endoderm. Ectoderm-derived tissue stem cells include neural stem cells existing in the brain, epidermal stem cells existing in the skin, follicular stem cells and pigment stem cells. Mesoderm-derived tissue stem cells include vascular stem cells, hematopoietic stem cells and mesenchymal stem cells observed in bone marrow and blood. Endoderm-derived tissue stem cells are mainly present in organs, including liver stem cells, pancreatic stem cells, and intestinal epithelial stem cells. In addition, germ line stem cells are present in the testes and the ovaries. In a preferred embodiment of the present invention, mesoderm-derived stem cells may be employed. In a more preferred embodiment of the present invention, bone marrow cells (e.g., hematopoietic stem cells) may be employed.

Tissue stem cells are separated into categories of sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like. In one embodiment of the present invention, it was unexpectedly revealed that bone marrow cells are preferable.

In a preferred embodiment of the present invention, bone marrow cells may be used, as it is, as a cell resource, or alternatively, a particular enriched or purified cell group (e.g., tissue stem cells) may be employed as a cell resource.

As used herein, the term “regeneration” refers to the recovery of injured tissue or organs to the original condition, and is also called pathological regeneration. The body of an organism may lose a part of its organs or may be heavily injured by traumas or diseases in its life time. In this case, whether or not the injured organ can be regenerated varies among organs (or among animal species). The branch of medicine that permits organs (or tissue), which cannot be naturally regenerated, to undergo regeneration so as to recover the function, is regenerative medicine. Whether or not tissue has regenerated, can be determined based on whether or not the function is improved. Mammals have the capability of regenerating tissue and organs to some degree (e.g., regeneration of skin, liver, and blood). However, the tissue of certain organs, such as heart, lung, brain, and the like have poor abilities to undergo regeneration. It has been believed that once such tissue is injured, the function cannot be recovered. Therefore, conventionally, when the heart is injured, heart transplant is substantially the only measure for the treatment of the organ.

It had been presumed since a long time ago that stem cells are present in organs having a high level of regenerative ability. This presumption was proved by experimental bone marrow transplantation using animal models. Subsequent studies have demonstrated that stem cells in bone marrow are resources for regeneration of all kinds of blood cells. It has also been demonstrated that stem cells are present in organs having a high level of regenerative ability, such as bone marrow, skin, and the like. In addition, although it was long believed that the brain cannot be regenerated, it has been demonstrated that stem cells are present in the brain, the heart, and the like. It has been clarified that stem cells are present in all organs in the body and play a role in regeneration of the organs to some extent. Stem cells present in each tissue have plasticity to an extent beyond expectation, so that stem cells in one organ may be used in regeneration of another organ. Therefore, the expansion function found in the present invention is effective for various tissue stem cells and the number of applications of the present invention is infinite.

As used herein, the terms “differentiation” or “cell differentiation” refers to a phenomenon that two or more types of cells having qualitative differences in form and/or function occur in a daughter cell population derived from the division of a single cell. Therefore, “differentiation” includes a process during which a population (family tree) of cells, which do not originally have a specific detectable feature, acquire a feature, such as production of a specific protein, or the like. At present, cell differentiation is generally considered to be a state of a cell in which a specific group of genes in the genome are expressed. Cell differentiation can be identified by searching for intracellular or extracellular agents or conditions which elicit the above-described state of gene expression. Differentiated cells are stable in principle. Particularly, animal cells which have been once differentiated are rarely differentiated into other types of cells. Therefore, “undifferentiated” refers to a state of a cell which does not have a difference in morphology and functionality.

As used herein, the term “maintain” in relation to an undifferentiated state refers to keeping pluripotency. Therefore, maintenance of an undifferentiated state can be assessed by determing whether or not pluripotency is kept.

As used herein, the term “expansion” in relation to stem cells indicates that the number of stem cells is increased while maintaining the autoexpansion ability and pluripotency of the stem cell. Although the expansion phenomenon of stem cells has been known, no molecule which specifically causes such an expansion has been found. Therefore, the present invention is the first to provide such a specific molecule. As used herein, pluripotency may be at any level as long as expansion can be maintained. For example, totipotency, pluripotency in a narrow sense (as possessed by tissue stem cells) and the like are illustrated herein. The present invention is not limited to this.

As used herein, the term “differentiated cell” refers to a cell having a specialized function and form (e.g., muscle cells, neurons, etc.). Unlike stem cells, differentiated cells have no or little pluripotency. Examples of differentiated cells include epidermic cells, pancreatic parenchymal cells, pancreatic duct cells, hepatic cells, blood cells, cardiac muscle cells, skeletal muscle cells, osteoblasts, skeletal myoblasts, neurons, vascular endothelial cells, pigment cells, smooth muscle cells, fat cells, bone cells, cartilage cells, and the like. Therefore, in one embodiment of the present invention, if a certain material cell can be treated by an agent of the present invention (e.g., active STAT5 polypeptide or nucleic acid) to obtain differentiated cells, such as a leucocyte or the like, the differentiated cell may also be within the scope of the present invention.

Cells used as a resource of the present invention can be differentiated into myocardial cells by treatment with a polypeptide, nucleic acid, composition, kit and/or medicament of the present invention.

As used herein, the term “pluripotency” refers to a nature of a cell, i.e., an ability to differentiate into one or more, preferably two or more, tissues or organs. Typically, the pluripotency of a cell is limited as the cell is developed, and in an adult, cells constituting a tissue or organ rarely alter to different cells, where the pluripotency is lost. Such alteration typically occurs in pathological conditions, and is called metaplasia. However, mesenchymal cells tend to easily undergo metaplasia, i.e., alter to other mesenchymal cells, with relatively simple stimuli. Therefore, mesenchymal cells have a high level of pluripotency. Therefore, cells having pluripotency may be preferable in the present invention, though not required.

As used herein, one type of pluripotency is “totipotency”, which refers to an ability to be differentiated into all kinds of cells which constitute an organism. The idea of pluripotency encompasses totipotency. An example of a totipotent cell is a fertilized ovum. Note that totipotency may be clearly separated from pluripotency. The former indicates an ability to be differentiated into all kinds of cells while the latter indicates an ability to be committed into a plurality of types of cells but not all types. An ability to be differentiated into only one type of cell is called “unipotency”.

As used herein, totipotency and pluripotency can be determined based on the number of days which has passed after fertilization. For example, for mouse, totipotency is distinguished from pluripotency with about Day 8 after fertilization as a borderline. Although not wishing to be bound by theory, for mouse, cells develop over time after fertilization as follows. On Day 6.5 after fertilization (also represented by E6.5), a primitive streak appears on the one side of an epiblast, clarifying the future anteroposterior axis of the embryo. The primitive streak indicates the future posterior end of the embryo, extending across the ectoderm to reach the distal end of the cylinder. The primitive streak is an area in which cell movement takes place. As a result, the future endoderm and mesoderm are formed. By E7.5 a head process appears ahead of the node, in which a notochord, and a future endoderm (lower layer) and a neural plate (upper layer) around the notochord, are formed. The node appears around E6.5 and moves backward, so that the axial structure is formed from the head to the tail. By E8.5 the embryo is elongated and a large head lamella mostly consisting of the anterior neural plate is formed at the anterior end of the embryo. Segments are formed at a rate of one per 1.5 hours from E8 from the head to the tail. After this stage, cells no longer exhibit totipotency or develop into an individual even if they are brought back to the placenta, except for dedifferentiation. Before this stage, cells have totipotency without any particular treatment. Thus, this stage is a branch point of totipotency. Therefore, it is difficult to establish ES cells from embryos after this point. In other words, it is possible to establish cells, typically called EG (germ cell-derived) cells, from embryos after this point. Also, in this context this point can be said to be a branch point.

Cells used in the present invention may be derived from any organism (e.g., vertebrates and invertebrates). Preferably, the organism is a vertebrate, and more preferably a mammal (e.g., the order Primates, Rodentia, etc.). Even more preferably, the organism is the order Primates. Most preferably, the organism is human.

As used herein, the term “within an organism” or “in vivo” refers to an inside of an organism. The term “within an organism” or “in vivo” refers to a position, at which a tissue or organ of interest is to be placed, in a particular context.

As used herein, the term “in vitro” indicates that a part of an organism is excised or isolated “outside the organism” (e.g., in a test tube) for the purposes of various research. This term is in contrast to the term “in vivo”.

As used herein, by the term “ex vivo” it is meant that a target cell for gene introduction has been isolated from a subject, and a therapeutic gene or agent is introduced in vitro into the cell, and the cell is returned to the subject. An expansion agent of the present invention is also useful for ex vivo therapy.

As used herein, the term “subject” refers to an organism which is treated according to the present invention, also called “patient”. A patient or a subject may be preferably a human.

As used herein, the term “recipient” refers to an individual which receives a graft, also called “host”. In contrast, an individual which provides a graft is called “donor”.

(Cellular Physiologically Active Substance)

As used herein, the term “cellular physiologically active substance” refers to a substance capable of acting on a cell, which may be employed with an expansion agent. Cellular physiologically active substances include cytokines and growth factors. A cellular physiologically active substance may be naturally-occurring or synthesized. Preferably, a cellular physiologically active substance is one that is produced by a cell or one that has a function similar thereto. As used herein, a cellular physiologically active substance may be in the form of a protein or a nucleic acid or in other forms. In actual practice, cytokines are typically proteins.

The term “cytokine” is used herein in the broadest sense in the art and refers to a physiologically active substance which is produced from a cell and acts on the same or different cell. Cytokines are generally proteins or polypeptides having a function of controlling an immune response, regulating the endocrine system, regulating the nervous system, acting against a tumor, acting against a virus, regulating cell growth, regulating cell differentiation, or the like. Cytokines are herein in the form of a protein or a nucleic acid or in other forms. In actual practice, cytokines are typically proteins.

The term “growth factor” or “cell growth factor” are used herein interchangeably and each refers to a substance which promotes or controls cell growth. Growth factors are also called “proliferation factor” or “development factor”. Growth factors may be added to cell or tissue culture medium, substituting for serum macromolecules. It has been revealed that a number of growth factors have a function of controlling differentiation in addition to a function of promoting cell growth. Examples of cytokines representatively include, but are not limited to, interleukins, chemokines, hematopoietic factors such as colony stimulating factors, a tumor necrosis factor, interferons, a platelet-derived growth factor (PDGF), an epidermal growth factor (EGF), a fibroblast growth factor (FGF), a hepatocyte growth factor (HGF), an endothelial cell growth factor (VEGF), cardiotrophin, and the like, which have proliferative activity.

Cellular physiologically active substances, such as cytokines, growth factors, and the like, typically have redundancy in function, Accordingly, reference herein to a particular cytokine or growth factor by one name or function also includes any other names or functions by which the factor is known to those of skill in the art, as long as the factor has the activity of a cellular physiologically active substance for use in the present invention. Cytokines or growth factors can be used in a preferred embodiment of the present invention as long as they have preferable activity as described herein.

In the present invention, any cellular physiologically active substance may be used. In a preferred embodiment of the present invention, as a cellular physiologically active substance, a cytokine or growth factor having hematopoietic activity, colony stimulating activity, or cell proliferative activity. Examples of a cytokine having hematopoietic activity or colony stimulating activity include a leukemia inhibitory factor (LIF), a granulocyte macrophage colony stimulating factor (GM-CSF), a macrophage colony stimulating factor (M-CSF), a granulocyte colony stimulating factor (G-CSF), a multi-CSF (IL-3), erythropoietin (EPO), c-kit ligand (SCF), and the like. Examples of a growth factor having cell proliferative activity include a platelet-derived growth factor (PDGF), an epidermal growth factor (EGF), a fibroblast growth factor (FGF), a hepatocyte growth factor (HGF), an endothelial cell growth factor (VEGF), an insulin-like growth factor (IGF), and the like. In a preferred embodiment of the present invention, a cellular physiologically active substance (e.g., a cytokine or a growth factor) having cell proliferative activity may be used. In a preferred embodiment, such a cellular physiologically active substance includes SCF, TPO, and Flt-3L.

Cellular physiologically active substances, such as cytokines and growth factors, can also be divided into categories in accordance with their receptors (e.g., cytokine receptors, etc.), Cytokine receptors are divided into non-kinase type and kinase type. Examples of the non-kinase type include G-protein binding type receptors, an NGF/TNF receptor family, an IFN receptor family, a cytokine receptor superfamily, and the like. Examples of the kinase type include a growth factor type receptor (tyrosine kinase type, such as c-met (for HGF)), a TGFβ receptor family (serine/threonine kinase type), and the like. In some cases, cellular physiologically active substances share a receptor subunit. Therefore, a cytokine or growth factor, which shares a receptor subunit with the above-described preferable cytokines or growth factors, may be a preferable cytokine or growth factor.

Cellular physiologically active substances, such as cytokines and growth factors, may also be divided into categories in accordance with homology comparison when they are provided in the form of a protein or a nucleic acid. Therefore, in a preferred embodiment of the present invention, a cellular physiologically active substance having homology to a preferable cellular physiologically active substance of the present invention may be used. Such a cellular physiologically active substance has at least about 30% homology to a control cellular physiologically active substance when BLAST is employed to perform comparison with default parameters, preferably about 35%, about 40%, about 45%, about 50%, about 55%, about 60%, about 65%, about 70% about 75%, about 80%, about 85%, about 90%, about 95%, and about 99% homology.

(Method for Producing Mutant Polypeptide)

Amino acid deletion, substitution or addition (including fusion) of the polypeptide of the present invention (e.g., STAT5, an active form thereof, or an activating agent therefor, and the like) can be carried out by a site-specific mutagenesis method which is a well known technique. One or several amino acid deletions, substitutions or additions can be carried out in accordance with methods described in Molecular Cloning, A Laboratory Manual, Second Edition, Cold Spring Harbor Laboratory Press (1989); Current Protocols in Molecular Biology, Supplement 1 to 38, John Wiley & Sons (1987-1997); Nucleic Acids Research, 10, 6487 (1982); Proc. Natl. Acad. Sci., USA, 79, 6409 (1982); Gene, 34, 315 (1985); Nucleic Acids Research, 13, 4431 (1985); Proc. Natl. Acad. Sci USA, 82, 488 (1985); Proc. Natl. Acad. Sci., USA, 81, 5662 (1984); Science, 224, 1431 (1984); PCT WO85/00817(1985); Nature, 316, 601 (1985); and the like.

(Medicament)

A polypeptide, nucleic acid, and medicament of the present invention, and a differentiated cell or differentiated cell composition prepared using the same may be provided in any formulation form which is suitable for transfer into organisms. Examples of such a formulation form include, but are not limited to, liquid drug, injection drug, sustained-release drug, and the like. Administration methods may herein include oral administration and parenteral administration (e.g., intravenous, intramuscular, subcutaneous, intradermal, mucosal, intrarectal, vaginal, topical to an affected site, to the skin, etc.), direct administration to affected portions, and the like. A prescription for such administration may be provided in any formulation form. Such a formulation form includes liquid formulations, injections, sustained preparations, and the like. When systemically administered, the medicament for use in the present invention may be in the form of a pyrogen-free, pharmaceutically acceptable aqueous solution. The preparation of such pharmaceutically acceptable compositions, with due regard to pH, isotonicity, stability and the like, is within the skill of the art.

Solvents for use in preparing a medicament of the present invention may be either aqueous or nonaqueous. Vehicles for a medicament of the present invention may contain other components for modifying or maintaining pH, osmolarity, viscosity, transparency, color, sterility, stability, isotonicity, disintegration rate, or smell of the medicament. Similarly, a composition of the present invention may contain other components for modifying or maintaining the release rate of an effective component or absorption or permeability of an effective component.

When formulated as a medicament or a medicament composition, the present invention may be prepared for storage by mixing a sugar chain composition having the desired degree of purity with optional physiologically acceptable carriers, excipients, or stabilizers (Japanese Pharmacopeia 14th ed. or the latest version; Remington's Pharmaceutical Sciences, 18th Edition, A. R. Gennaro, ed., Mack Publishing Company, 1990; and the like), in the form of lyophilized cake or aqueous solutions.

Examples of pharmaceutical acceptable agents include, but are not limited to, antioxidants, preservatives, colorants, flavoring agents, diluents, emulsifiers, suspending agents, solvents, fillers, bulking agents, buffers, delivery vehicles, and/or pharmaceutical adjuvants. Representatively, a medicament of the present invention is administered in the form of a composition comprising an active component of the present invention (e.g., a polypeptide, a nucleic acid, or the like) with at least one physiologically acceptable carrier, excipient or diluent. For example, an appropriate vehicle may be injection solution, physiological solution, or artificial cerebrospinal fluid, which can be supplemented with other substances which are commonly used for compositions for parenteral delivery. Acceptable carriers, excipients or stabilizers used herein preferably are nontoxic to recipients and are preferably inert at the dosages and concentrations employed, and preferably include phosphate, citrate, or other organic acids; antioxidants (e.g., ascorbic acid); low molecular weight polypeptides; proteins (e.g., serum albumin, gelatin, or immunoglobulins); hydrophilic polymers (e.g., polyvinylpyrrolidone); amino acids (e.g., glycine, glutamine, asparagine, arginine or lysine); monosaccharides, disaccharides, and other carbohydrates (glucose, mannose, or dextrins); chelating agents (e.g., EDTA); sugar alcohols (e.g., mannitol or sorbitol); salt-forming counterions (e.g., sodium); and/or nonionic surfactants (e.g., Tween, pluronics or polyethylene glycol (PEG)).

Injection drugs can be prepared using techniques well known in the art. For example, an agent of the present invention is dissolved in an appropriate solvent (physiological saline, buffer (e.g., PBS, etc.), sterilized water, etc.), followed by filter sterilization using a filter, or the like. Thereafter, the solution is placed into a sterile container (e.g., an ampoule, or the like). Thus, an injection drug can be prepared. The injection drug may contain a commonly used pharmaceutical carrier if required. An administration method using a non-invasive catheter may be employed. Examples of appropriate carriers include neutral buffered saline or saline mixed with serum albumin. Preferably, the product is formulated as a lyophilizate using appropriate excipients (e.g., sucrose). Other standard carriers, diluents, and excipients may be included as desired. Other exemplary compositions comprise Tris buffer of about pH 7.0-8.5, or acetate buffer of about pH 4.0-5.5, which may further include sorbitol or a suitable substitute therefor. The pH of the solution or various other pH should be selected based on the relative solubility of an active component of the present invention (e.g., a polypeptide, a nucleic acid, etc.).

A protocol of preparing a formulation of the present invention is known in the art as described in, for example, the Japanese Pharmacopeia, the U.S. Pharmacopeia, Pharmacopeias of other countries, and the like. Therefore, those skilled in the art can determine the amount of a polypeptide or a cell to be administered in view of the present specification without undue experimentation.

In one embodiment, a composition and medicament of the present invention may be provided in a sustained-release form. When administered in a sustained-release form, an active component (e.g., a nucleic acid or a polypeptide) is effective if the efficacy thereof can be expected over along time due to sustained release. Any sustained-released dosage form may be used in the present invention. Examples of sustained-release dosage forms include, but are not limited to, rod-like formulations (e.g., pellet-like, cylinder-like, needle-like formulations, etc.), tablet formulations, disk-like formulations, sphere-like formulations, sheet-like formulations, and the like. Methods for preparing sustained-release dosage forms are well known in the art, as described in, for example, the Japanese Pharmacopeia, the U.S. Pharmacopeia, Pharmacopeias of other countries, and the like. Examples of a method for producing sustained-release drugs include, but are not limited to, a method using disaggregation of a drug from a complex, a method for preparing an aqueous suspended liquid drug, a method for preparing an oil injection liquid or oil suspended injection liquid, a method for preparing an emulsified injection liquid (o/w or w/o type emulsified injection liquid, or the like), and the like.

In another embodiment of the present invention, it is intended that other pharmaceutical agents are administered in combination with an agent of the present invention. Such pharmaceutical agents may be any medicament known in the art, such as, for example, any pharmaceutical agent known in pharmacology (e.g., antibiotics, etc.). Two or more of such pharmaceutical agents may be simultaneously used. These pharmaceutical agents are described in, for example, the Japanese Pharmacopeia (latest edition), the U.S. Pharmacopeia (latest edition), Pharmacopeias (latest edition) of other countries, and the like.

In another embodiment, a medicament or composition of the present invention may contain two or more kinds of cells prepared by a method of the present invention. When two or more kinds of cells are employed, the cells may have similar properties or may be derived from similar origins, or may have different properties or may be derived from different origins.

The amount of a polypeptide, nucleic acid, compound, medicament, or cell used in the treatment method of the present invention can be easily determined by those skilled in the art with reference to the purpose of use, the subject's age, size, sex, and case history, the form or type of the polypeptide, nucleic acid, compound, medicament, or cell, and the like.

The frequency of the compound of the present invention which is applied to a subject is also determined by those skilled in the art with respect to the purpose of use, the subject's age, size, sex, case history, the progression of the therapy, and the like. Examples of the frequency include once per day to once per several months (e.g., once per week to once per month). Preferably, administration is performed once per week to once per month with reference to the progression.

### BEST MODE FOR CARRYING OUT THE INVENTION

Hereinafter, preferred embodiments of the present invention will be described. The following embodiments are provided for a better understanding of the present invention and the scope of the present invention should not be limited to the following description. It will be clearly appreciated by those skilled in the art that variations and modifications can be made without departing from the scope of the present invention with reference to the specification.

The present invention provides a composition and method for maintaining the expansion or pluripotency of a stem cell (e.g., a hematopoietic stem cell or the like). There has been no known method capable of maintaining the pluripotency and self-replication ability of a stem cell, such as a hematopoietic stem cell or the like, more significantly than naturally-occurring stem cells. Thus, the present invention provides an astonishing effect in the art.

In one aspect, the present invention provides a composition for maintaining the expansion, pluripotency, or self-replication ability of a stem cell, comprising active STAT5. Preferably, the stem cell is a hematopoietic stem cell. Active STAT5 may be active STAT5A or active STAT5B. Preferably, active STAT5 is a peptide comprising a sequence set forth in SEQ ID NO:2, 4, 6 or 8, or a variant thereof, in which at least one serine, threonine, or tyrosine residue is phosphorylated. The phosphorylated site may be preferably a tyrosine residue, more preferably a tyrosine residue at a C-terminal side (e.g., position 694 in SEQ ID NOs:2 and 6, position 699 in SEQ ID NOs:4 and 8). Examples of the preferably phosphorylated residue include, but are not limited to, threonine at position 461, tyrosine at position 694 and serine at position 726 in SEQ ID NO:2, and corresponding threonine, tyrosine and serine, and the like.

Active STAT5 may be:

(a) a polypeptide consisting of an amino acid sequence set forth in SEQ ID NO:2, 4, 6 or 8, or a fragment thereof;

(b) a polypeptide comprising an amino acid sequence set forth in SEQ ID NO:2, 4, 6 or 8 having at least one mutation selected from at least one amino acid substitution, addition and deletion, and having biological activity;

(c) a polypeptide encoded by an allelic variant of a base sequence set forth in SEQ ID NO:1, 3, 5 or 7;

(d) a polypeptide which is a species homolog of an amino acid sequence set forth in SEQ ID NO:2, 4, 6 or 8; or

(e) a polypeptide having an amino acid sequence having at least 70% identity to any one of the polypeptides (a) to (d), and having biological activity. The active STAT5 may be a dimer. The dimer may be a homodimer or a heterodimer. A homodimer is preferable.

Whether or not a certain agent is active STAT5 or not can be determined by detecting the nuclear transfer ability and/or STAT5 consensus sequence-binding ability thereof. Methods for producing active STAT5 are known in the art. For example, active STAT5 can be produced by expressing a nucleic acid molecule encoding consistently or transiently active STAT5 using techniques well known in the art. Alternatively, inactive STAT5 is prepared by genetic manipulation, purification of naturally-occurring molecules, or organic synthesis, followed by specific phosphorylation with a kinase, such as JAK or the like (preferably, JAK1, JAK2, JAK3, or the like which are known to phosphorylate STAT5 (including STAT5A and STAT5B)). Phosphorylated STAT5 is normally dimerized, so that the activity thereof is maintained.

In a preferred embodiment, in the active STAT5 of the present invention, a glutamic acid residue at position 150 and/or a histidine residue at position 298 and/or a serine residue at position 710 in SEQ ID NO:2 or 6, or residues corresponding thereto may be substituted with respective different amino acids, preferably glycine and/or arginine and/or phenylalanine, respectively. Preferably, mutation may be introduced into two of the three residues. Alternatively, the serine residue at position 710 may be preferably substituted with phenylalanine. More preferably, a polypeptide having an amino acid sequence set forth in SEQ ID NO:10 or 13 may be used as a preferable active STAT5 of the present invention.

In a certain embodiment, the active STAT5 of the present invention may be consistently active or transiently active. “Consistent” or “transient” can be appropriately selected by those skilled in the art in accordance with the situation.

Preferably, the active STAT5 of the present invention has an amino acid sequence having at least about 70% homology to SEQ ID NO:2, 4, 6 or 8. More preferably, the active STAT5 of the present invention may have an amino acid sequence having at least about 80% homology to SEQ ID NO:2, 4, 6 or 8, more preferably at least about 85%, even more preferably at least about 90%, still more preferably at least about 95%, and still even more preferably about 99%. In another embodiment, the active STAT5 of the present invention has an amino acid sequence having at least about 80% homology to a sequence defining a helix and/or a loop therebetween in SEQ ID NO:2 or 4, more preferably at least about 85%, even more preferably at least about 90%, still more preferably at least about 95%, and still even more preferably about 99%. Most preferably, the active STAT5 of the present invention has a sequence set forth in SEQ ID NO:2, 4, 6, 8, 10 or 13.

In a preferred embodiment, the present invention may comprise an additional cellular physiologically active substance. Examples of the cellular physiologically active substance include, but are not limited to, interleukins, chemokines, hematopoietic factors (e.g., colony stimulating factors), tumor necrosis factors, interferons, platelet-derived growth factors (PDGFs), epithelial growth factors (EGFs), fibroblast growth factors (FGFs), hepatocyte growth factors (HGFs), vascular endothelial growth factors (VEGFs), and the like, which have growth activity. In a certain embodiment, the cellular physiologically active substance is preferably selected from the group consisting of SCF, TPO, and Flt-3L. This is because SCF, TPO, and Flt-3L have been reported to play a role in maintaining the undifferentiated state. Preferably, all of SCF, TPO, and Flt-3L may be employed.

A composition of the present invention may be used as a medicament composition. The composition can comprise a pharmaceutically acceptable carrier.

In another aspect, the present invention provides a composition for maintaining the expansion, pluripotency, or self-replication ability of a stem cell, comprising STAT5 and an agent capable of activating STAT5. STAT5 may be:

(a) a polypeptide consisting of an amino acid sequence set forth in SEQ ID NO:2, 4, 6 or 8, or a fragment thereof;

(b) a polypeptide comprising an amino acid sequence set forth in SEQ ID NO:2, 4, 6 or 8 having at least one mutation selected from at least one amino acid substitution, addition and deletion, and having biological activity;

(c) a polypeptide encoded by an allelic variant of a base sequence set forth in SEQ ID NO:1, 3, 5 or 7;

(d) a polypeptide which is a species homolog of an amino acid sequence set forth in SEQ ID NO:2, 4, 6 or 8; or

(e) a polypeptide having an amino acid sequence having at least 70% identity to any one of the polypeptides (a) to (d), and having biological activity.

The STAT5 activating agent may be known in the art or may be newly produced. Whether or not a certain agent is capable of activating STAT5 can be determined by, for example, causing the agent to act on STAT5 or a variant thereof and subjecting the resultant STAT5 or variant to an assay for investigating active STAT5. Examples of the STAT5 activating agent may include agents selected from the JAK family (e.g., JAK1, JAK2, and JAK3) or a variant thereof.

In another aspect, the present invention provides a composition for maintaining the expansion, pluripotency, or self-replication ability of a stem cell, comprising a nucleic acid molecule encoding active STAT5. The present invention also provides a composition for maintaining the expansion, pluripotency, or self-replication ability of a stem cell, comprising a nucleic acid molecule encoding STAT5 and an agent capable of phosphorylating at least one serine, threonine, or tyrosine residue of STAT5. Preferably, the nucleic acid molecule may comprise a nucleic acid sequence encoding STAT5 which forms a dimer.

In one embodiment, the nucleic acid molecule encoding active STAT5 may be:

(a) a polynucleotide having a base sequence set forth in SEQ ID NO:1, 3, 5 or 7 or a sequence fragment thereof;

(b) a polynucleotide encoding a polypeptide consisting of an amino acid sequence set forth in SEQ ID NO:2, 4, 6 or 8, or a fragment thereof;

(c) a polynucleotide encoding a variant polypeptide having an amino acid sequence set forth in SEQ ID NO:2, 4, 6 or 8 having at least one mutation selected from the group consisting of at least one amino acid substitution, addition and deletion, and having biological activity;

(d) a polynucleotide which is an allelic variant of DNA consisting of a base sequence set forth in SEQ ID NO:2, 4, 6 or 8;

(e) a polynucleotide encoding a species homolog of a polypeptide consisting of an amino acid sequence set forth in SEQ ID NO:2, 4, 6 or 8;

(f) a polynucleotide encoding a polypeptide hybridizable to any one of the polynucleotides (a) to (e) under stringent conditions, and having biological activity; or

(g) a polynucleotide consisting of a base sequence having at least 70% identity to any one of the polynucleotides (a) to (e) or a complementary sequence thereof, and having biological activity.

A composition of the present invention in the form of a nucleic acid molecule may be used as it is or as it is contained in a plasmid vector (e.g., a retrovirus vector or the like), The composition may further comprise a carrier, such as a liposome or the like.

A nucleic acid molecule encoding active STAT5 for use in the present invention has at least 70% nucleic acid sequence homology to SEQ ID NO:1, 3, 5 or 7. More preferably, the nucleic acid molecule encoding the active STAT5 of the present invention may have at least about 80% nucleic acid sequence homology to a sequence set forth in SEQ ID NO:1, 3, 5 or 7, more preferably at least about 85%, even more preferably at least about 90%, still more preferably at least about 95%, and still even more preferably about 99%. In another embodiment, the nucleic acid molecule encoding the active STAT5 of the present invention comprises a sequence having at least about 80% nucleic acid sequence homology to a sequence encoding a helix and/or a loop therebetween of a sequence set forth in SEQ ID NO:1, 3, 5 or 7, more preferably at least about 85%, even more preferably at least about 90%, still more preferably at least about 95%, and still even morepreferablyabout99%. Mostpreferably,thenucleicacid molecule encoding the active STAT5 of the present invention has a sequence set forth in SEQ ID NO:1, 3, 5, 7, 9 or 12.

The nucleic acid sequence may be modified so as to encode a molecule which forms a dimer. In a non-limiting example, the sequence is a sequence set forth in SEQ ID NO:9 or a modification thereof (glutamic acid at position 150 is substituted with glycine, histidine at position 298 is substituted with arginine, and serine at position 710 is substituted with phenylalanine). Glutamic acid at position 150, histidine at position 298, and serine at position 710 may be substituted with any amino acids as long as the sequence encodes a molecule capable of forming a dimer. Therefore, those skilled in the art can determine substitutions as appropriate while assessing the activation ability of STAT5. In another aspect, the present invention provides a method for maintaining the expansion or pluripotency of a stem cell. The method comprises A) providing a stem cell; and B) providing a composition of the present invention to the stem cell.

Therefore, in a preferred embodiment, in active STAT5 encoded by a nucleic acid molecule of the present invention, a glutamic acid residue at position 150 and/or a histidine residue at position 298 and/or a serine residue at position 710 in SEQ ID NO:2 or 6, or residues corresponding thereto may be substituted with respective different amino acids, preferably glycine and/or arginine and/or phenylalanine, respectively. Preferably, mutation may be introduced into two of the three residues. Alternatively, the serine residue at position 710 may be preferably substituted with phenylalanine. More preferably, a polypeptide having an amino acid sequence set forth in SEQ ID NO:10 or 13 may be used as a preferable active STAT5 of the present invention.

In a preferred embodiment, the stem cell is selected from the group consisting of embryonic stem cells and tissue stem cells. More preferably, the stem cell may be a hematopoietic stem cell.

In a preferred embodiment, the method of the present invention further comprises administering an additional cellular physiologically active substance. In a preferred embodiment, the additional cellular physiologically active substance is selected from the group consisting of SCF, TPO, and Flt-3L.

In one embodiment, the present invention provides a method for maintaining the expansion, pluripotency, or self-replication ability of a stem cell. The method comprises the steps of A) providing a stem cell; and B) providing active STAT5 to the stem cell. The stem cell may be preferably a hematopoietic stem cell. The active STAT5 may be one which is herein described in the preferred embodiments.

In one embodiment, the present invention provides a method for maintaining the expansion, pluripotency, or self-replication ability of a stem cell. The method comprises the steps of A) providing a stem cell; B) providing STAT5 to the stem cell; and C) activating the STAT5. An agent for the activation may be, but is not limited to, a member selected from the JAK family or a variant thereof. The activating agent can be selected by those skilled in the art in view of the present specification as appropriate. The STAT5 may be one which is herein described in the preferred embodiments.

In one aspect, the present invention provides use of active STAT5 for maintaining the expansion, pluripotency, or self-replication ability of a stem cell. The active STAT5 may be one which is herein described in the preferred embodiments. Preferably, the stem cell may be a hematopoietic stem cell.

In one aspect, the present invention provides use of STAT5 for maintaining the expansion, pluripotency, or self-replication ability of a stem cell. The STAT5 may be one which is herein described in the preferred embodiments. Preferably, the stem cell may be a hematopoietic stem cell.

In one aspect, the present invention provides use of STAT5 and an agent capable of activating the STAT5 for maintaining the expansion, pluripotency, or self-replication ability of a stem cell. The activating agent may be, but is not limited to, a member selected from the JAK family or a variant thereof. The activating agent can be selected by those skilled in the art in view of the present specification as appropriate.

In another aspect, the present invention provides a cell obtained by the method of the present invention, tissues and organs obtained by the cell.

In a preferable aspect of the present invention, the present invention provides a medicament composition comprising a cell obtained by the method of the present invention. The medicament composition may comprise a pharmaceutical carrier and an additional effective component if required.

In another aspect of the present invention, the present invention provides a method for treatment or prophylaxis of a disease or a disorder in need of a stem cell or a differentiated cell derived from the stem cell. The method comprises the steps of A) administering a cell obtained by the present invention to a subject in need of the treatment or prophylaxis.

In another aspect of the present invention, the present invention provides use of a cell obtained by the present invention for treatment or prophylaxis of a disease or a disorder in need of a stem cell or a differentiated cell derived from the stem cell. Techniques for use of such a cell are well known in the art and can be selected by those skilled in the art as appropriate.

In another aspect, the present invention provides a stem cell prepared by the method for maintaining the expansion, pluripotency, or self-replication ability of the stem cell. The method can be used in vivo or in vitro. The stem cell prepared by the method of the present invention can be secured in a prescribed quality and can be prepared in a large quantity, which are not conventionally acquired features. Therefore, the stem cell of the present invention has a preferable therapeutic effect.

Thus, the present invention can provide stem cells more efficiently. This effect is advantageous over conventional techniques and is considerably useful.

Hereinafter, the present invention will be described by way of examples. Examples described below are provided only for illustrative purposes. Accordingly, the scope of the present invention is not limited except as by the appended claims.

### EXAMPLES

### Example 1

### Preparation of Retrovirus Vectors

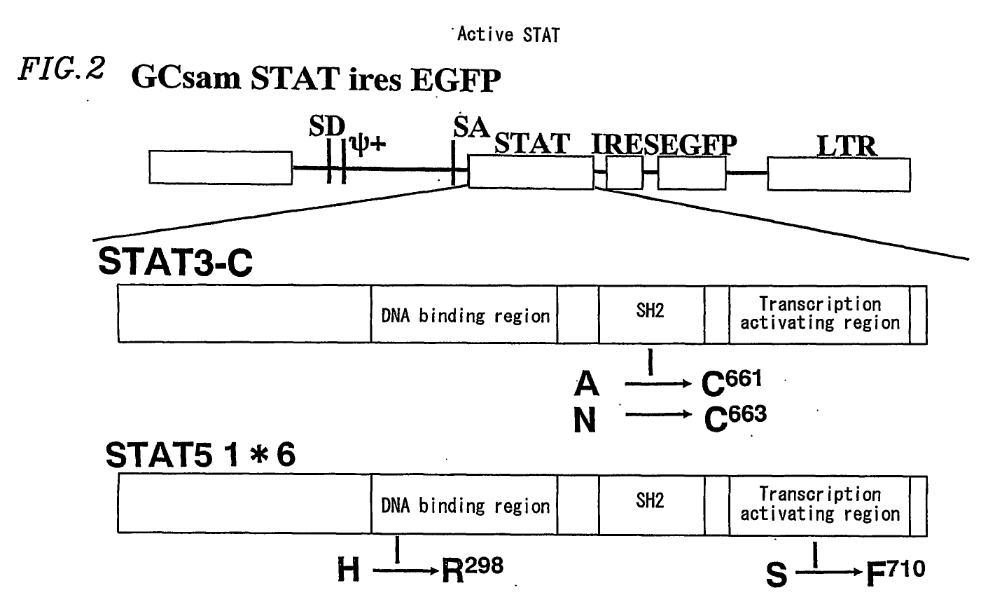

In Example 1, a construct which is known to consistently form a dimer was used as active STAT5. The structure of the construct is shown in FIG. 2. The construct is called GCsam STAT IRES EGFP. This STAT5 is a variant of STAT5 (SEQ ID NO:9 (nucleic acid sequence) and SEQ ID NO:10 (amino acid sequence)), called STAT5A 1*6. It is advantageous that the variant consistently forms a dimer and thus consistently exhibits the activity of active STAT5.

A packaging cell, 293 gp cell, was transfected with GC-sam-STAT5A 1*6-IRES-EGFP (a retrovirus vector GCsam-IRES-EGFP having a STAT5A 1*6 gene inserted) or VSV-g (vesicular stomatitis virus G protein) envelope expression vector. Culture supernatant was collected (see FIG. 2).

Culture was carried out in Dulbecco's Modified Eagle Medium (DMEM) containing 10% fetal bovine serum (FBS), supplemented with 1× penicillin (100 U/ml), 1× streptomycin (100 μg/ml) in a humified 10% CO₂ atmosphere at 37° C. Representatively, the cells were subcultured every 3 days at a ratio of 1:4 to 1:10.

Transfection of the 293 gp cell was carried out as follows. Representatively, 7×10⁶ 293 gp cells were plated on 15-cm plates. After 24 hours, the cells were transfected using a phosphate calcium method as follows.

Water was aliquoted into 15-ml Eppendorf tubes at 1.215 ml/tube. 135 μl of 2.5 M calcium chloride was added to DNA solution (vector plasmid (GCsam STAT IRES EGFP, FIG. 2, 45 μg) and envelope plasmid (vsv-g, 20 μg)) and the solution was mildly mixed. 1.35 ml of 2×BBS was added to the DNA solution, followed by incubation at room temperature for 20 min. The resultant mixture was added to cell culture medium. Resultant virus producing cells were cultured in a humified 5% CO₂ atmosphere at 37° C. for 16 hours. Thereafter, the cells were transferred to fresh cell culture medium.

48 hours after transfection (transfer to fresh cell culture medium), culture supernatant was collected with a 10-ml syringe (15 ml/15-cm plate) and was filtered through 0.45-μmembrane (HTuffryn, Pall Gelman Laboratory (Ann Arbor, Mich., USA)) so as to remove the virus producing cells and cell debris. Alternatively, virus supernatant was centrifuged at 19,400 rpm for 2 hours, resulting in about 100-fold enrichment. Filtering virus preparations were immediately used for infection or stored at −80° C.

### Example 2

### Preparation of Cells

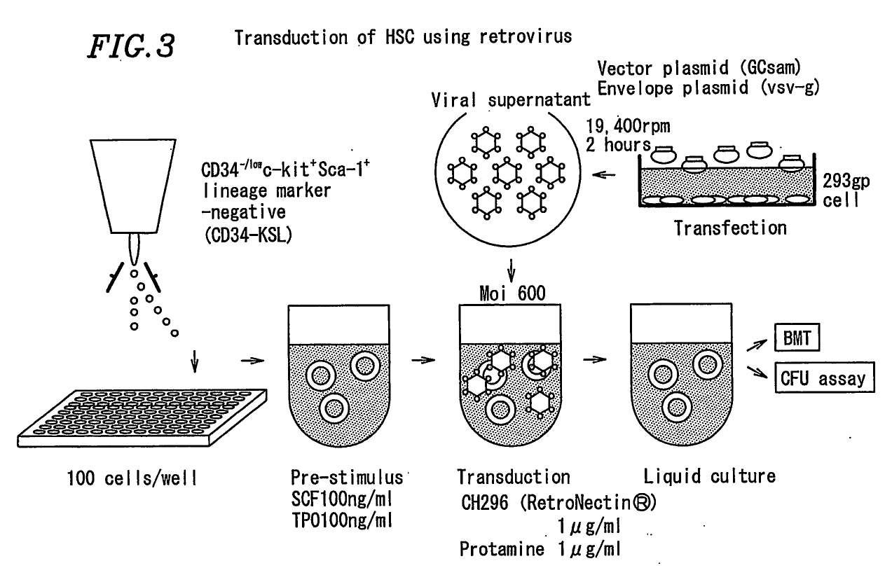

Bone marrow liquid was collected from 8-10 week old C57BL/6 Ly5.1 mice, followed by specific gravity centrifugation to separate mononuclear cells (see FIG. 3). The mononuclear cells were stained using various monoclonal antibodies (anti-CD34, anti-c-Kit, anti-Sca-1, Lineage-marker mixture. (CD4, CD8, B220, Gr-1, Mac-1, and Ter119)). Hematopoietic stem cell fractions detected with CD34^{-/low}c-kit⁺Sca-1⁺Lineage-marker⁻ (CD34⁻KSL) were plated onto 96-well multititer plates at a rate of 100 cells/well using a FACS Vantage cell sorter (Becton Dickinson). Serum-free culture medium X-vivo-10 (BioWhittaker) was used at a rate of 200 μl/well, supplemented with SCF (Stem cell factor) and TPO (thrombopoietin) as cytokines at a rate of 100 ng/ml for each (see FIG. 3).

### Example 3

### Infection with Viruses

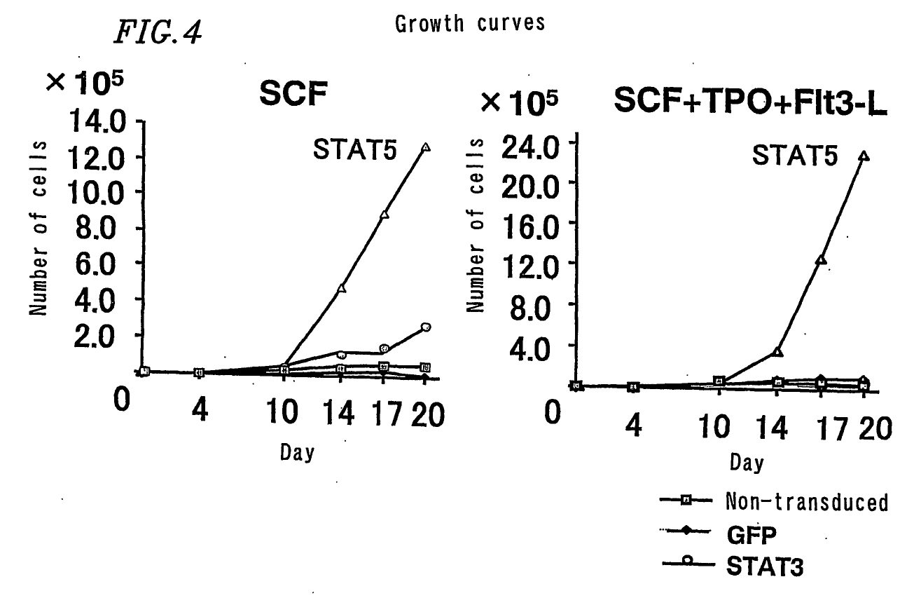

Hematopoietic stem cells harvested in Example 2 were cultured for 24 hours. Thereafter, the cells were infected with the enriched virus at an Multiplicity of infection (MOI) of 600. In this case, infection was assisted by addition of CH296 (RetroNectin (registered trademark): Takara Shuzo) and protamine (Sigma) at a rate of 1 microgram/ml for each. 24 hours after adding the virus, the cells were washed with 5 ml of X-vivo-10 and resuspended in medium containing SCF only or a combination of SCF, TPO and Flt3L (Flt3 ligand) (100 ng/ml), followed by culture for 7-9 days. The results are shown in FIG. 4.

### Example 4

### Colony Assay

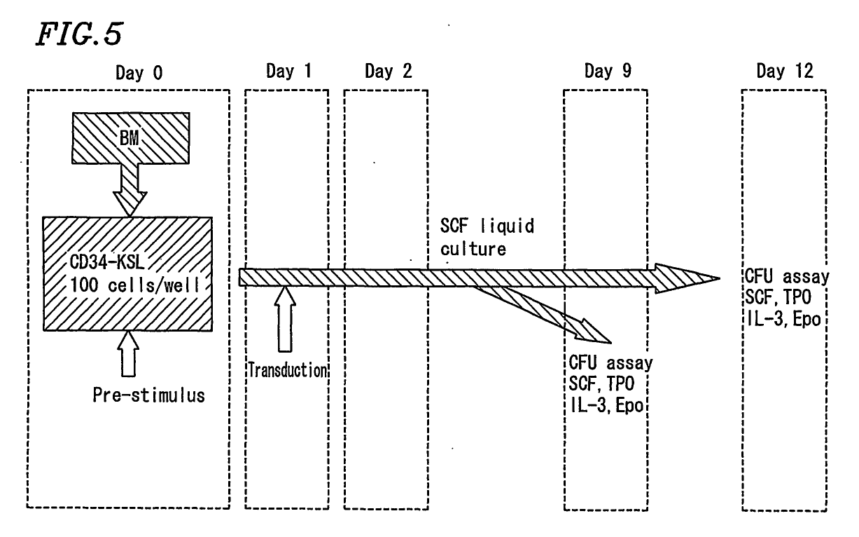

After viral infection and 7-9 day culture in medium containing SCF only or a combination of SCF, TPO and Flt3L (Flt3 ligand), a part of the cells were suspended in methylcellulose medium (Methocult: Stem Cell Technologies, Inc.), followed by culture for 10 days (FIG. 5). In this case, the following cytokines were employed: SCF (20 ng/ml); TPO (100 ng/ml); IL-3 (interleukin-3, 20 ng/ml); and Epo (erythropoietin, 5 units/ml). After 10 days, the number and type of colonies were assessed (FIG. 5).

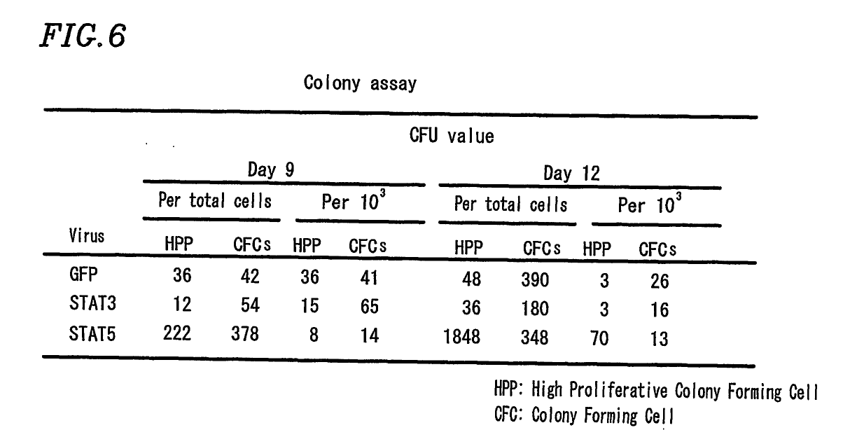

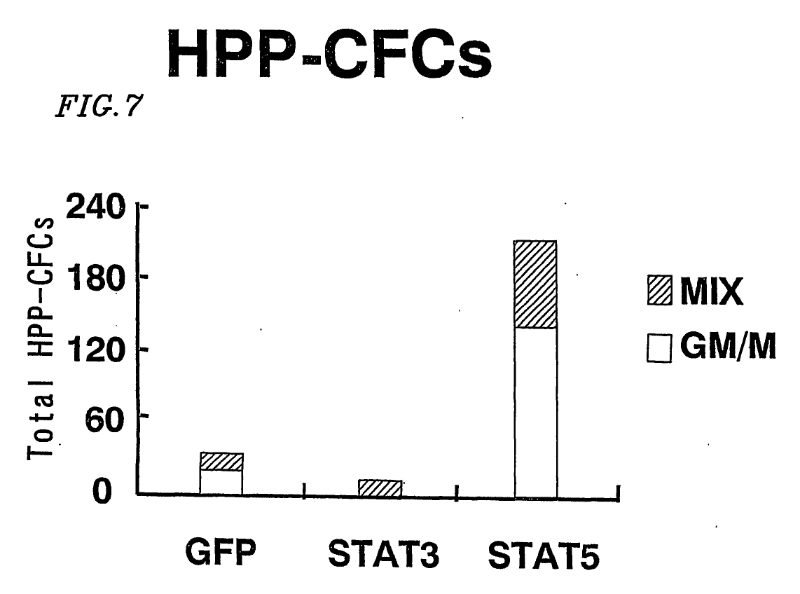

The results were shown in FIG. 6. Referring to FIG. 6, GFP and STAT3 (see their structures shown in FIG. 2), which were used as controls, had substantially no pluripotency maintaining function. In contrast, active STAT5 significantly maintained the pluripotency and self-replication ability of the cells. FIG. 7 is a bar chart showing the results. As can be seen clearly from FIG. 7, active STAT5 had a significant effect.

Thus, it was demonstrated that active STAT5 has a function of maintaining the pluripotency and self-replication ability of stem cells, i.e., a function of expanding stem cells. This finding had not been conventionally known. In particular, it was remarkable that active STAT3 had substantially no effect while only active STAT5 had the effect.

### Example 5

### Bone Marrow Transplantation

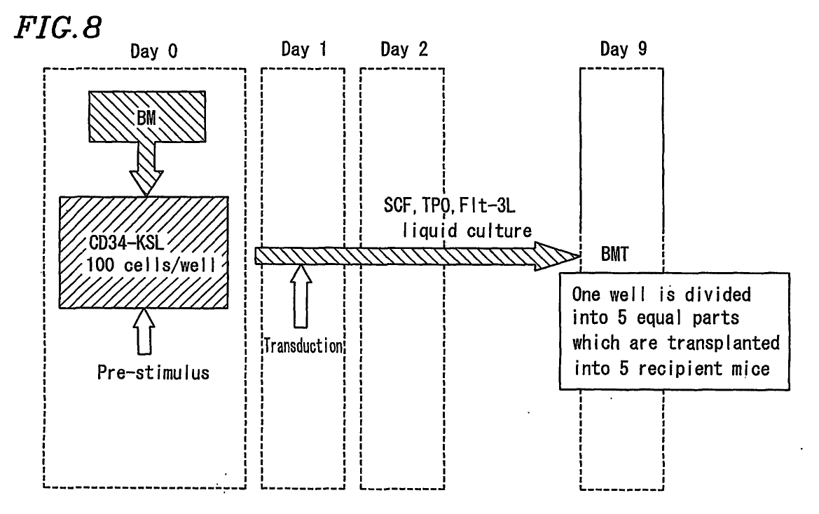

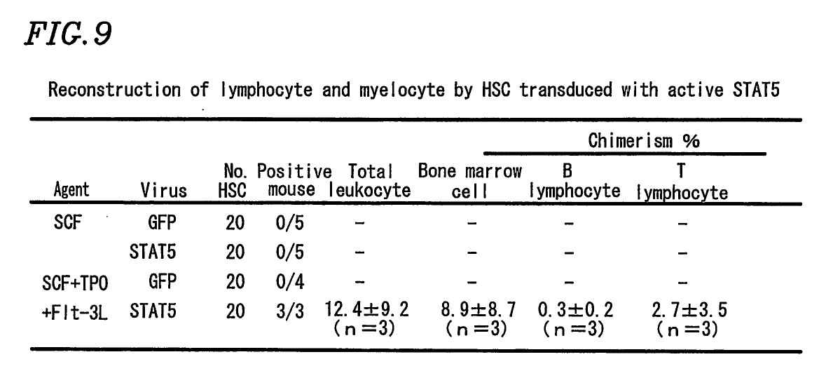

After viral infection and 7-9 day culture in medium containing SCF only or a combination of SCF, TPO and Flt3L (Flt3 ligand), the cells were divided into 5 equal parts, each of which was mixed with 2×10⁵ C57BL/6 Ly5.2 mouse bone marrow cells. The cells were injected into C57BL/6 Ly5.2 mice which had been subjected to a lethal dose of radiation, via the tail vein. After transplantation of the cells, the peripheral blood was collected at fixed intervals and the chimerism of the transplanted cells in the peripheral blood was observed using a flow cytometer (see FIG. 8), The cells having the introduced gene expressed GFP. By quantitating the GFP positive cells, the ratio of the contribution of the STAT5-introduced cells to bone marrow reconstruction after bone marrow transplantation could be assessed. The results are shown in FIG. 9. As shown in FIG. 9, it was demonstrated that by adding SCF, TPO and Flt-3L as well as active STAT5, the activity of hematopoietic stem cells can be significantly secured. Note that even if active STAT5 is employed singly, the activity of stem cells can be maintained, however, the effect is weak. Therefore, it was found that addition of a cellular physiologically active substance is preferable.

### Example 6

### Direct Introduction of Protein

Instead of the above-described use of the gene, active STAT5 was directly employed to determine whether or not the protein can maintain the ability of stem cells.

As active STAT5, the above-described STAT5A 1*6 was employed. This gene product was prepared using techniques well known in the art.

The activity of the prepared STAT5A 1*6 was assessed by a gel shift assay using a double-stranded oligonulceotide comprising a base sequence (5′-GATCCGAATTCCAGGAATTCAGATC-3′) (SEQ ID NO:11) containing a prolactin responsive element (PRE) used as a STAT5-binding base sequence. A solution of the prepared protein was reacted with the nucleic acid molecule comprising the base sequence, followed by polyacrylamide gel electrophoresis and separation. Thereafter, the formation of a complex of the agent with the nucleic acid molecule containing the consensus sequence was identified. Thus, it was demonstrated that active STAT5 can maintain the activity of stem cells.

Active STAT5 was subjected to a colony assay and a bone marrow transplantation assay. It was demonstrated that substantially the same effect was obtained, although the effect was weak.

### Example 7

### Effect of Another Active STAT5 Variant

Instead of the above-described active STAT5A, the effect of another modified STAT5 variant was studied. In the variant, E (glutamic acid) at position 150 in SEQ ID NO:6 was substituted with G (glycine) and S (serine) at position 710 was substituted with F (phenylalanine). The sequence of the variant was set forth in SEQ ID NOs:12 (nucleic acid sequence) and 13 (amino acid sequence). The variant was designated STAT5A 1*7. The variant was inserted into a construct as obtained in Example 1 instead of STAT5A 1*6. It was revealed that the variant consistently forms a dimer and always exhibits the activity of active STAT5.

A packaging cell, 293 gp cell, was transfected with GC-sam-STAT5A 1*7-IRES-EGFP (a retrovirus vector GCsam-IRES-EGFP having an inserted STAT5A 1*7 gene) or VSV-g (vesicular stomatitis virus G protein) envelope expression vector. Culture supernatant was collected (see FIG. 2).

Culture was carried out in Dulbecco's Modified Eagle Medium (DMEM) containing 10% fetal bovine serum (FBS), supplemented with 1× penicillin (100 U/ml), 1× streptomycin (100 μg/ml) in a humified 10% CO₂ atmosphere at 37° C. Representatively, the cells were subcultured every 3 days at a ratio of 1:4 to 1:10.

Transfection of the 293 gp cell was carried out as follows. Representatively, 7×10⁶ 293 gp cells were plated on 15-cm plates. After 24 hours, the cells were transfected using a phosphate calcium method as follows.

Water was aliquoted into 15-ml Eppendorf tubes at 1.215 ml/tube. DNA solution (45 μg of a vector plasmid) and 20 μg of an envelope plasmid (vsv-g)) were added to the water. Thereafter, 135 μl of 2.5 M calcium chloride was added to the solution and the solution was mildly mixed. 1.35 ml of 2×BBS was added to the DNA solution, followed by incubation at room temperature for 20 min. The resultant mixture was added to cell culture medium. Resultant virus producing cells were cultured in a humified 5% CO₂ atmosphere at 37° C. for 16 hours. Thereafter, the cells were transferred to fresh cell culture medium.

48 hours after transfection (transfer to fresh cell culture medium), culture supernatant was collected with a 10-ml syringe (15 ml/15-cm plate) and was filtered through 0.45-μm membrane (HTuffryn, Pall Gelman Laboratory (Ann Arbor, Mich., USA)) so as to remove the virus producing cells and cell debris. Alternatively, virus supernatant was centrifuged at 19,400 rpm for 2 hours, resulting in about 100-fold enrichment. Filtering virus preparations were immediately used for infection or stored at −80° C.

Cells as prepared in Example 2 were infected with the product as described in Example 3, followed by a colony assay as described in Example 4. As a result, it was demonstrated that STAT5A 1*7 has substantially the same level of activity as that of STAT5A 1*6.

### Example 8

### Effect of Another Variant on Bone Marrow Transplantation

The effect of STAT5A 1*7 on bone marrow transplantation was assessed (particularly, ability to reconstruct bone marrow). The protocol was described in Example 5.

4 weeks after transplantation, the chimerism of cells, which had been transformed with STAT5A 1*7, in bone marrow was described in a table below. In the table, GFP was employed as a control and STAT5 A1*6 and STAT5 A1*7 prepared in Example 1 are compared with each other.

TABLE

| Virus No. | HSC (Number of Cells) | Mouse | Chimerism |
|---|---|---|---|
| GFP | 20 | 1 | 0.02% |
| GFP | 20 | 2 | 0.02% |
| GFP | 20 | 3 | 0.2% |
| STAT5 1*6 | 20 | 1 | 0.5% |
| STAT5 1*6 | 20 | 2 | 5.9% |
| STAT5 1*6 | 20 | 3 | 35.3% |
| STAT5 1*7 | 20 | 1 | 28.2% |
| STAT5 1*7 | 20 | 2 | 57.9% |

### Example 9

### Effect of Other STAT5

Instead of the above-described active STAT5A, active STAT5B and human active STAT5A (variants of the above-described STAT5 1*6 and STAT5 1*7, having corresponding mutations) are used to carry out the same experiments as those described in Examples 1 to 5. As a result, it is found that active STAT5 similarly maintains the undifferentiated state and self-replication ability. Therefore, it is revealed active STAT5 has the effect of the present invention as long as the variant keeps the ability of STAT5 (particularly, downstream signal transduction).

Although certain preferred embodiments have been described herein, it is not intended that such embodiments be construed as limitations on the scope of the invention except as set forth in the appended claims. All patents, published patent applications and publications cited herein are incorporated by reference as if set forth fully herein.

### Example 10

### Screening for a substance Capable of Activating STAT5

Although active STAT5 can be employed in the present invention, an agent capable of activating STAT5 maybe useful. Therefore, in Example 10, an attempt is made to screen for an agent capable of activating Example 10. An agent capable of activating STAT5 can be screened for and identified by preparing inactive STAT5 and determining whether or not the inactive STAT5 is activated by a candidate agent.

Such a selected agent can promote activation of STAT5 and therefore can be used as an agent for expanding stem cells. Such a substance could be small compounds that activate STAT5 by binding to and dimerizing inactive STAT5.

Whether or not a substance is active STAT5 can be determined by investigating the ability to enter the nucleus of a cell and/or the ability to bind to a STAT5 consensus sequence. In Example 10, by confirming these abilities of a substance, it can be determined whether or not the substance is active STAT5. Specifically, in Example 10, cells are transfected with a STAT5 gene and a candidate agent is inserted into the cells; and thereafter, the cells are immunologically stained with anti-STAT5 antibodies so as to determine the localization of STAT5 in the nucleus. When the candidate agent is a protein, the agent is directly inserted into the cell, and thereafter, the transfer of the agent into the nucleus can be determined by immunogically staining the agent using antibodies specific to the agent. To achieve this, techniques well known in the art are available.

To detect ability to bind to the STAT5 consensus sequence, a gel shift assay is employed, in which a double-stranded oligonulceotide comprising a base-sequence (5′-GATCCGAATTCCAGGAATTCAGATC-3′) (SEQ ID NO:11) containing a prolactin responsive element (PRE) as a STAT5 binding base sequence is used. Specifically, a candidate compound is inserted into cells; a certain inactive STAT5 is reacted with a nucleic acid molecule comprising the base sequence, followed by polyacrylamide gel electrophoresis and separation. The binding ability can be detected by identifying the formation of a complex of the inactive STAT5 with a nucleic acid molecule comprising the consensus sequence. Typically, if the formation of a complex can be significantly confirmed, the agent can be determined to have the same function as that of active STAT5.

Transcription activation is detected using a bovine β casein promoter comprising the PRE sequence as described above. A luciferase gene is linked as a reporter gene downstream of the β casein promoter. Cells are simultaneously transfected with the reporter gene and a STAT5 gene. Thereafter, the luciferase activity is measured. Alternatively, when the agent is applied directly to the gene, cells are transfected with only the reporter gene and the luciferase activity is measured. Thereby, whether or not a molecule of interest is active STAT5 can be determined. If the luciferase activity is increased, the molecule can be recognized as having transcription activation ability. While not wishing to be bound by the following, the recognition can be made by performing statistical processing and determining the statistical significance.

Whether or not a molecule of interest is active STAT5 can be sufficiently determined by detecting a significant level of activity using at least one of the above-described methods. If activity is confirmed by a luciferase reporter assay, it is indirectly proven that the molecule is transferred into nuclei and binds to DNA. Therefore, the determination can be basically made by observing a signal from the luciferase reporter.

### Example 11

### Utilization of STAT5 Activating Agent

The agent capable of activating STAT5 that is prepared in Example 10 can be used to expand stem cells. If the agent is administered into stem cells which express inactive STAT5 and the stem cells are cultured, it is found that the stem cells are expanded.

(Effect of the Invention)

The present invention provides a composition for maintaining the expansion, pluripotency, or self-replication ability of a stem cell (e.g., hematopoietic stem cells).

Although certain preferred embodiments have been described herein, it is not intended that such embodiments be construed as limitations on the scope of the invention except as set forth in the appended claims. All patents, published patent applications and publications cited herein are incorporated by reference as if set forth fully herein.

### INDUSTRIAL APPLICABILITY

The present invention is the first to provide a composition for expanding stem cells, particularly hematopoietic stem cells. The composition is considerably useful when hematopoietic stem cells and differentiated cells therefrom are applied to various treatments. The composition may be employed as a medicament, and thus, is particularly useful in the pharmaceutical industry.

1. A composition for maintaining the expansion, pluripotency, or self-replication ability of a stem cell, comprising active STAT5.

2. A composition according to claim 1, wherein the stem cell is a hematopoietic stem cell.

3. A composition according to claim 1, wherein the active STAT5 is active STAT5A or active STAT5B.

4. A composition according to claim 1, wherein the active STAT5 is active STAT5A.

5. A composition according to claim 1, wherein the active STAT5 is:
(a) a polypeptide consisting of an amino acid sequence set forth in SEQ ID NO:2, 4, 6 or 8, or a fragment thereof;
(b) a polypeptide comprising an amino acid sequence set forth in SEQ ID NO:2, 4, 6 or 8 having at least one mutation selected from at least one amino acid substitution, addition and deletion, and having biological activity;
(c) a polypeptide encoded by an allelic variant of a base sequence set forth in SEQ ID NO:1, 3, 5 or 7;
(d) a polypeptide which is a species homolog of an amino acid sequence set forth in SEQ ID NO:2, 4, 6 or 8; or
(e) a polypeptide having an amino acid sequence having at least 70% identity to any one of the polypeptides (a) to (d), and having biological activity; and
wherein in the active STAT5, at least one serine, threonine, or tyrosine residue is phosphorylated.

6. A composition according to claim 1, wherein the active STAT5 is a homodimer or a heterodimer of:
(a) a polypeptide consisting of an amino acid sequence set forth in SEQ ID NO:2, 4, 6 or 8, or a fragment thereof;
(b) a polypeptide comprising an amino acid sequence set forth in SEQ ID NO:2, 4, 6 or 8 having at least one mutation selected from at least one amino acid substitution, addition and deletion, and having biological activity;
(c) a polypeptide encoded by an allelic variant of a base sequence set forth in SEQ ID NO:1, 3, 5 or 7;
(d) a polypeptide which is a species homolog of an amino acid sequence set forth in SEQ ID NO:2, 4, 6 or 8; or
(e) a polypeptide having an amino acid sequence having at least 70% identity to any one of the polypeptides (a) to (d), and having biological activity.

7. A composition according to claim 1, wherein in the active STAT5, a tyrosine residue at position 694 in SEQ ID NO:2 or a tyrosine residue corresponding thereto is at least phosphorylated.

8. A composition according to claim 1, wherein the active STAT5 is a dimer.

9. A composition according to claim 1, wherein the active STAT5 has an amino acid residue substitution in at least one position selected from the group consisting of position, 150, 298, and 710 in SEQ ID NO:2 or 6.

10. A composition according to claim 1, wherein in the active STAT5, a histidine residue at position 298 and/or a serine residue at position 710 in SEQ ID NO:2 or 6, or residues corresponding thereto, are substituted with arginine and/or phenylalanine, respectively.

11. A composition according to claim 1, wherein in the active STAT5, a glutamic acid residue at position 150 and/or a serine residue at position 710 in SEQ ID NO:2 or 6, or residues corresponding thereto, are substituted with glycine and/or phenylalanine, respectively.

12. A composition according to claim 1, wherein in the active STAT5, a serine residue at position 710 in SEQ ID NO:2 or 6 is substituted with phenylalanine.

13. A composition according to claim 1, wherein the active STAT5 has an amino acid sequence set forth in SEQ ID NO:10 or 13.

14. A composition according to claim 1, wherein the active STAT5 is consistently active or transiently active.

15. A composition according to claim 1, further comprising a cellular physiologically active substance.

16. A composition according to claim 15, wherein the cellular physiologically active substance is selected from the group consisting of SCF, TPO, and Flt-3L.

17. A composition according to claim 15, wherein the cellular physiologically active substance contains SCF, TPO, and Flt-3L.

18. A composition according to claim 1, further comprising a pharmaceutically acceptable carrier.

19. A composition for maintaining the expansion, pluripotency, or self-replication ability of a stem cell, comprising STAT5 and an agent capable of activating STAT5.

20. A composition according to claim 19, wherein the STAT5 is:
(a) a polypeptide consisting of an amino acid sequence set forth in SEQ ID NO:2, 4, 6 or 8, or a fragment thereof;
(b) a polypeptide comprising an amino acid sequence set forth in SEQ ID NO:2, 4, 6 or 8 having at least one mutation selected from at least one amino acid substitution, addition and deletion, and having biological activity;
(c) a polypeptide encoded by an allelic variant of a base sequence set forth in SEQ ID NO:1, 3, 5 or 7;
(d) a polypeptide which is a species homolog of an amino acid sequence set forth in SEQ ID NO:2, 4, 6 or 8; or
(e) a polypeptide having an amino acid sequence having at least 70% identity to any one of the polypeptides (a) to (d), and having biological activity.

21. A composition according to claim 19, wherein the STAT5 activating agent is a member selected from the JAK family or a variant thereof.

22. A composition for maintaining the expansion, pluripotency, or self-replication ability of a stem cell, comprising a nucleic acid molecule encoding active STAT5.

23. A composition according to claim 22, wherein the nucleic acid molecule encoding active STAT5 contains a nucleic acid sequence encoding STAT5 which forms a dimer.

24. A composition according to claim 22, wherein the nucleic acid molecule encoding active STAT5 is:
(a) a polynucleotide having a base sequence set forth in SEQ ID NO:1, 3, 5 or 7 or a sequence fragment thereof;
(b) a polynucleotide encoding a polypeptide consisting of an amino acid sequence set forth in SEQ ID NO:2, 4, 6 or 8, or a fragment thereof;
(c) a polynucleotide encoding a variant polypeptide having an amino acid sequence set forth in SEQ ID NO:2, 4, 6 or 8 having at least one mutation selected from the group consisting of at least one amino acid substitution, addition and deletion, and having biological activity;
(d) a polynucleotide which is an allelic variant of DNA consisting of a base sequence set forth in SEQ ID NO:2, 4, 6 or 8;
(e) a polynucleotide encoding a species homolog of a polypeptide consisting of an amino acid sequence set forth in SEQ ID NO:2, 4, 6 or 8;
(f) a polynucleotide encoding a polypeptide hybridizable to any one of the polynucleotides (a) to (e) under stringent conditions, and having biological activity; or
(g) a polynucleotide consisting of a base sequence having at least 70% identity to any one of the polynucleotides (a) to (e) or a complementary sequence thereof, and having biological activity.

25. A composition according to claim 22, wherein the active STAT5 has an amino acid residue substitution in at least one position selected from the group consisting of position, 150, 298, and 710 in SEQ ID NO:2 or 6.

26. A composition according to claim 22, wherein in the active STAT5, a histidine residue at position 298 and/or a serine residue at position 710 in SEQ ID NO:2 or 6, or residues corresponding thereto, are substituted with arginine and/or phenylalanine, respectively.

27. A composition according to claim 22, wherein in the active STAT5, a glutamic acid residue at position 150 and/or a serine residue at position 710 in SEQ ID NO:2 or 6, or residues corresponding thereto, are substituted with glycine and/or phenylalanine, respectively.

28. A composition according to claim 22, wherein in the active STAT5, a serine residue at position 710 in SEQ ID NO:2 or 6 is substituted with phenylalanine.

29. A composition according to claim 22, wherein the active STAT5 has an amino acid sequence set forth in SEQ ID NO:10 or 13.

30. A composition according to claim 22, wherein the nucleic acid molecule is contained in a vector.

31. A composition according to claim 22, wherein the nucleic acid molecule is contained in a retrovirus vector.

32. A composition according to claim 22, wherein the nucleic acid molecule has a sequence set forth in SEQ ID NO:9.

33. A composition for expanding a stem cell, comprising a nucleic acid molecule encoding STAT5 and an agent capable of activating the STAT5.

34. A composition according to claim 33, wherein the nucleic acid molecule encoding active STAT5 is:
(a) a polynucleotide having a base sequence set forth in SEQ ID NO:1, 3, 5 or 7 or a sequence fragment thereof;
(b) a polynucleotide encoding a polypeptide consisting of an amino acid sequence set forth in SEQ ID NO:2, 4, 6 or 8, or a fragment thereof;
(c) a polynucleotide encoding a variant polypeptide having an amino acid sequence set forth in SEQ ID NO:2, 4, 6 or 8 having at least one mutation selected from the group consisting of at least one amino acid substitution, addition and deletion, and having biological activity;
(d) a polynucleotide which is an allelic variant of DNA consisting of a base sequence set forth in SEQ ID NO:2, 4, 6 or 8;
(e) a polynucleotide encoding a species homolog of a polypeptide consisting of an amino acid sequence set forth in SEQ ID NO:2, 4, 6 or 8;
(f) a polynucleotide encoding a polypeptide hybridizable to any one of the polynucleotides (a) to (e) under stringent conditions, and having biological activity; or
(g) a polynucleotide consisting of a base sequence having at least 70% identity to any one of the polynucleotides (a) to (e) or a complementary sequence thereof, and having biological activity.

35. A composition according to claim 33, wherein the nucleic acid molecule is contained in a vector.

36. A composition according to claim 33, wherein the nucleic acid molecule is contained in a retrovirus vector.

37. A composition according to claim 33, wherein the STAT5 activating agent is a member selected from the JAK family or a variant thereof.

38. A method for maintaining the expansion, pluripotency, or self-replication ability of a stem cell, comprising the steps of:
A) providing a stem cell; and
B) providing active STAT5 to the stem cell.

39. A method for maintaining the expansion, pluripotency, or self-replication ability of a stem cell, comprising the steps of:
A) providing a stem cell;
B) providing STAT5 to the stem cell; and
C) activating the STAT5.

40. A method for preparing a stem cell in a predetermined quantity, comprising the steps of:
A) providing a stem cell; and
B) providing active STAT5, or STAT5 and an agent capable of activating the STAT5, to the stem cell.

41. A cell, obtained by a method according to any one of claims 38 to 40.

42. Use of active STAT5 for maintaining the expansion, pluripotency, or self-replication ability of a stem cell.

43. Use of STAT5 for maintaining the expansion, pluripotency, or self-replication ability of a stem cell.

44. Use of STAT5 and an agent capable of activating the STAT5 for maintaining the expansion, pluripotency, or self-replication ability of a stem cell.

45. A cell, obtained by treating a stem cell with active STAT5, or STAT5 and an agent capable of activating the STAT5.

46. A tissue, obtained by a cell obtained by treating a stem cell with active STAT5, or STAT5 and an agent capable of activating the STAT5.

47. An organ, obtained by a cell obtained by treating a stem cell with active STAT5, or STAT5 and an agent capable of activating the STAT5.

48. A medicament composition, comprising a cell obtained by treating a stem cell with active STAT5, or STAT5 and an agent capable of activating the STAT5.

49. A method for treatment or prophylaxis of a disease or a disorder in need of a stem cell or a differentiated cell derived therefrom, comprising the steps of:
A) administering to a subject in need of the treatment or prophylaxis a cell obtained by treating a stem cell with active STAT5, or STAT5 and an agent capable of activating the STAT5.

50. Use of a cell obtained by treating a stem cell with active STAT5, or STAT5 and an agent capable of activating the STAT5, for treatment or prophylaxis of a disease or a disorder in need of a stem cell or a differentiated cell derived therefrom.

51. Use of a cell obtained by treating a stem cell with active STAT5, or STAT5 and an agent capable of activating the STAT5, for production of a medicament for treatment or prophylaxis of a disease or a disorder in need of a stem cell or a differentiated cell derived therefrom.


SEQUENCE LISTING (13 sequences)

SEQ ID NO: 1 - DNA, 2385 bp, Homo sapiens
  CDS (1)..(2385)
atggcgggctggatccaggcccagcagctgcagggagacgcgctgcgccagatgcaggtgctgtacggccagcacttccccatcgaggtccggcactacttggcccagtggattgagagccagccatgggatgccattgacttggacaatccccaggacagagcccaagccacccagctcctggagggcctggtgcaggagctgcagaagaaggcggagcaccaggtgggggaagatgggtttttactgaagatcaagctggggcactacgccacgcagctccagaaaacatatgaccgctgccccctggagctggtccgctgcatccggcacattctgtacaatgaacagaggctggtccgagaagccaacaattgcagctctccggctgggatcctggttgacgccatgtcccagaagcaccttcagatcaaccagacatttgaggagctgcgactggtcacgcaggacacagagaatgagctgaagaaactgcagcagactcaggagtacttcatcatccagtaccaggagagcctgaggatccaagctcagtttgcccagctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacattcatcattgagaagcagcctcctcaggtcctgaagacccagaccaagtttgcagccaccgtacgcctgctggtgggcgggaagctgaacgtgcacatgaatcccccccaggtgaaggccaccatcatcagtgagcagcaggccaagtctctgcttaaaaatgagaacacccgcaacgagtgcagtggtgagatcctgaacaactgctgcgtgatggagtaccaccaagccacgggcaccctcagtgcccacttcaggaacatgtcactgaagaggatcaagcgtgctgaccggcggggtgcagagtccgtgacagaggagaagttcacagtcctgtttgagtctcagttcagtgttggcagcaatgagcttgtgttccaggtgaagactctgtccctacctgtggttgtcatcgtccacggcagccaggaccacaatgccacggctactgtgctgtgggacaatgcctttgctgagccgggcagggtgccatttgccgtgcctgacaaagtgctgtggccgcagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtccgtgtcctggtcccagttcaacagggagaacttgccgggctggaactacaccttctggcagtggtttgacggggtgatggaggtgttgaagaagcaccacaagccccactggaatgatggggccatcctaggttttgtgaataagcaacaggcccacgacctgctcatcaacaagcccgacgggaccttcttgttgcgctttagtgactcagaaatcgggggcatcaccatcgcctggaagtttgactccccggaacgcaacctgtggaacctgaaaccattcaccacgcgggatttctccatcaggtccctggctgaccggctgggggacctgagctatctcatctatgtgtttcctgaccgccccaaggatgaggtcttctccaagtactacactcctgtgctggctaaagctgttgatggatatgtgaaaccacagatcaagcaagtggtccctgagtttgtgaatgcatctgcagatgctgggggcagcagcgccacgtacatggaccaggccccctccccagctgtgtgcccccaggctccctataacatgtacccacagaaccctgaccatgtactcgatcaggatggagaattcgacctggatgagaccatggatgtggccaggcacgtggaggaactcttacgccgaccaatggacagtcttgactcccgcctctcgccccctgccggtcttttcacctctgccagaggctccctctcatga

SEQ ID NO: 2 - protein, 794 aa, Homo sapiens
MAGWIQAQQLQGDALRQMQVLYGQHFPIEVRHYLAQWIESQPWDAIDLDNPQDRAQATQLLEGLVQELQKKAEHQVGEDGFLLKIKLGHYATQLQKTYDRCPLELVRCIRHILYNEQRLVREANNCSSPAGILVDAMSQKHLQINQTFEELRLVTQDTENELKKLQQTQEYFIIQYQESLRIQAQFAQLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWDNAFAEPGRVPFAVPDKVLWPQLCEALNMKFKAEVQSNRGLTKENLVFLAQKLFNNSSSHLEDYSGLSVSWSQFNRENLPGWNYTFWQWFDGVMEVLKKHHKPHWNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPVLAKAVDGYVKPQIKQVVPEFVNASADAGGSSATYMDQAPSPAVCPQAPYNMYPQNPDHVLDQDGEFDLDETMDVARHVEELLRRPMDSLDSRLSPPAGLFTSARGSLS

SEQ ID NO: 3 - DNA, 2364 bp, Homo sapiens
  CDS (1)..(2364)
atggctgtgtggatacaagctcagcagctccaaggagaagcccttcatcagatgcaagcgttatatggccagcattttcccattgaggtgcggcattatttatcccagtggattgaaagccaagcatgggactcagtagatcttgataatccacaggagaacattaaggccacccagctcctggagggcctggtgcaggagctgcagaagaaggcagagcaccaggtgggggaagatgggtttttactgaagatcaagctggggcactatgccacacagctccagaacacgtatgaccgctgccccatggagctggtccgctgcatccgccatatattgtacaatgaacagaggttggtccgagaagccaacaatggtagctctccagctggaagccttgctgatgccatgtcccagaaacacctccagatcaaccagacgtttgaggagctgcgactggtcacgcaggacacagagaatgagttaaaaaagctgcagcagactcaggagtacttcatcatccagtaccaggagagcctgaggatccaagctcagtttggcccgctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacgttcatcattgagaagcagcctcctcaggtcctgaagacccagaccaagtttgcagccactgtgcgcctgctggtgggcgggaagctgaacgtgcacatgaaccccccccaggtgaaggccaccatcatcagtgagcagcaggccaagtctctgctcaagaacgagaacacccgcaatgattacagtggcgagatcttgaacaactgctgcgtcatggagtaccaccaagccacaggcacccttagtgcccacttcaggaatatgtccctgaaacgaattaagaggtcagaccgtcgtggggcagagtcggtgacagaagaaaaatttacaatcctgtttgaatcccagttcagtgttggtggaaatgagctggtttttcaagtcaagaccctgtccctgccagtggtggtgatcgttcatggcagccaggacaacaatgcgacggccactgttctctgggacaatgcttttgcagagcctggcagggtgccatttgccgtgcctgacaaagtgctgtggccacagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtctgtgtcctggtcccagttcaacagggagaatttaccaggacggaattacactttctggcaatggtttgacggtgtgatggaagtgttaaaaaaacatctcaagcctcattggaatgatggggccattttggggtttgtaaacaagcaacaggcccatgacctactcattaacaagccagatgggaccttcctcctgagattcagtgactcagaaattggcggcatcaccattgcttggaagtttgattctcaggaaagaatgttttggaatctgatgccttttaccaccagagacttctccattcggtccctagccgaccgcttgggagacttgaattaccttatctacgtgtttcctgatcggccaaaagatgaagtatactccaaatactacacaccagttccctgcgagtctgctactgctaaagctgttgatggatacgtgaagccacagatcaagcaagtggtccctgagtttgtgaacgcatctgcagatgccgggggcggcagcgccacgtacatggaccaggccccctccccagctgtgtgtccccaggctcactataacatgtacccacagaaccctgactcagtccttgacaccgatggggacttcgatctggaggacacaatggacgtagcgcggcgtgtggaggagctcctgggccggccaatggacagtcagtggatcccgcacgcacaatcgtga

SEQ ID NO: 4 - protein, 787 aa, Homo sapiens
MAVWIQAQQLQGEALHQMQALYGQHFPIEVRHYLSQWIESQAWDSVDLDNPQENIKATQLLEGLVQELQKKAEHQVGEDGFLLKIKLGHYATQLQNTYDRCPMELVRCIRHILYNEQRLVREANNGSSPAGSLADAMSQKHLQINQTFEELRLVTQDTENELKKLQQTQEYFIIQYQESLRIQAQFGPLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFESQFSVGGNELVFQVKTLSLPVVVIVHGSQDNNATATVLWDNAFAEPGRVPFAVPDKVLWPQLCEALNMKFKAEVQSNRGLTKENLVFLAQKLFNNSSSHLEDYSGLSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCESATAKAVDGYVKPQIKQVVPEFVNASADAGGGSATYMDQAPSPAVCPQAHYNMYPQNPDSVLDTDGDFDLEDTMDVARRVEELLGRPMDSQWIPHAQS

SEQ ID NO: 5 - DNA, 2382 bp, Mus musculus
  CDS (1)..(2382)
atggcgggctggattcaggcccagcagcttcagggagatgccctgcgccagatgcaagtgttgtatgggcagcatttccccatcgaggtccggcactacctggcccagtggatcgagagccagccgtgggatgctattgacttggataatccccaggaccgaggtcaggccacccaactcctggagggcctggtgcaggagctgcagaagaaggcggagcaccaggtgggggaagatgggtttttgctgaagatcaagctggggcactatgccacacagctccagaacacgtatgaccgctgtcccatggagctggttcgctgtatccgtcacattctgtacaacgaacagaggctggttcgcgaagccaacaattgcagctcccctgctggtgtcctggttgacgccatgtcccagaagcaccttcagatcaaccaaaggtttgaggagctgcgcctgatcacacaggacacggagaacgagctgaagaagctgcagcagacccaagagtacttcatcatccagtaccaggagagcctgcggatccaagctcagtttgcccagctgggccagctgaacccccaggagcgcatgagcagggagacggccctccagcagaagcaagtgtccctggagacctggctgcagcgagaggcacagacactgcagcagtaccgagtggagctggctgagaagcaccagaagaccctgcagctgctgcggaagcagcagaccatcatcctggacgacgagctgatccagtggaagcggagacagcagctggccgggaacgggggtccccccgagggcagcctggacgtgctgcagtcctggtgtgagaagctggccgagatcatctggcagaaccggcagcagatccgcagggctgagcacctgtgccagcagctgcccatcccaggccccgtggaggagatgctggctgaggtcaacgccaccatcacggacatcatctcagctctggtcaccagcacgttcatcatcgagaagcagcctcctcaggtcctgaagacccagaccaagtttgcggccaccgtgcgcctgctggtggggggaaagctgaatgtgcacatgaaccccccgcaggtgaaggcgaccatcatcagcgagcagcaggccaagtccctgctcaagaatgagaacacccgcaatgagtgcagcggcgagatcctgaacaactgttgcgtcatggagtaccaccaggccactggcacgctcagcgcccacttcagaaacatgtcactgaaaagaatcaagcgcgccgacaggcgtggtgcagagtcggtgacggaggagaagttcacagtcctgtttgagtctcagttcagcgttggcagcaacgagctggtgttccaggtgaagaccctgtccctccctgtggtcgttatcgtccatggcagccaggaccacaatgctactgccaccgtgctgtgggacaatgcctttgctgagccgggcagggtgccatttgctgtgcctgacaaggtgctgtggccgcagctgtgtgaagcgctcaacatgaaattcaaggctgaagtacagagcaaccggggcttgaccaaagagaacctcgtgttcctggcacagaaactgttcaacatcagcagcaaccacctcgaggactacaacagcatgtctgtgtcctggtcccagttcaaccgggagaacttgcccggctggaactacaccttctggcagtggttcgacggggtgatggaggtgctgaagaagcaccataagccccattggaatgatggggctatcctgggtttcgtgaacaagcaacaggcccacgacctgctcatcaacaagccggacgggaccttcctgctgcgcttcagtgactcggaaatcgggggcatcaccattgcttggaagtttgactctccggaccgaaacctctggaatctgaagccattcacgacgcgagatttctccattcggtccctggccgaccggctgggggacctgaactaccttatctacgtgttcccagaccgacccaaggacgaggtctttgccaagtattacactcctgtacttgcgaaagcagttgacggatacgtgaagccacagatcaagcaagtggtccctgagttcgtcaatgcatccacagatgccggagccagcgccacctacatggaccaggctccttccccagtcgtgtgccctcaacctcactacaacatgtacccacccaaccctgaccctgtccttgaccaagatggcgagtttgacctggatgagagcatggatgttgccaggcacgtggaagaacttttacgccggcccatggacagtctcgacgcccgcctctccccacctgctggtctcttcacctccgctagaagctccctgtcctga

SEQ ID NO: 6 - protein, 793 aa, Mus musculus
MAGWIQAQQLQGDALRQMQVLYGQHFPIEVRHYLAQWIESQPWDAIDLDNPQDRGQATQLLEGLVQELQKKAEHQVGEDGFLLKIKLGHYATQLQNTYDRCPMELVRCIRHILYNEQRLVREANNCSSPAGVLVDAMSQKHLQINQRFEELRLITQDTENELKKLQQTQEYFIIQYQESLRIQAQFAQLGQLNPQERMSRETALQQKQVSLETWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWDNAFAEPGRVPFAVPDKVLWPQLCEALNMKFKAEVQSNRGLTKENLVFLAQKLFNISSNHLEDYNSMSVSWSQFNRENLPGWNYTFWQWFDGVMEVLKKHHKPHWNDGAILGFVNKQQAHDLLINKPDGTFLLRFSDSEIGGITIAWKFDSPDRNLWNLKPFTTRDFSIRSLADRLGDLNYLIYVFPDRPKDEVFAKYYTPVLAKAVDGYVKPQIKQVVPEFVNASTDAGASATYMDQAPSPVVCPQPHYNMYPPNPDPVLDQDGEFDLDESMDVARHVEELLRRPMDSLDARLSPPAGLFTSARSSLS

SEQ ID NO: 7 - DNA, 2361 bp, Mus musculus
  CDS (1)..(2361)
atggctatgtggatacaggctcagcagctccagggcgatgcccttcaccagatgcaggccttgtacggccagcatttccccatcgaggtgcgacattatttatcacagtggatcgaaagccaagcctgggactcaatagatcttgataatccacaggagaacattaaggccacccagctcctggagggcctggtgcaggagctgcagaagaaggcggagcaccaggtgggggaagatgggtttttgctgaagatcaagctggggcactatgccacacagctccagagcacgtacgaccgctgccccatggagctggttcgctgtatccggcacattctgtacaacgaacagaggctggttcgcgaagccaacaacggcagctctccagctggaagtcttgctgacgccatgtcccagaagcaccttcagatcaaccaaacgtttgaggagctgcgcctgatcacacaggacacggagaacgagctgaagaagctgcagcagacccaagagtacttcatcatccagtaccaggagagcctgcggatccaagctcagtttgcccagctgggacagctgaacccccaggagcgcatgagcagggagacggccctccagcagaagcaagtgtccctggagacctggctgcagcgagaggcacagacactgcagcagtaccgagtggagctggctgagaagcaccagaagaccctgcagctgctgcggaagcagcagaccatcatcctggacgacgagctgatccagtggaagcggagacagcagctggccgggaacgggggtccccccgagggcagcctggacgtgctgcagtcctggtgtgagaagctggccgagatcatctggcagaatcggcagcagatccgcagggctgagcacctgtgccagcagctgcccatcccaggccccgtggaggagatgctggctgaggtcaacgccaccatcacggacatcatctcagccctggtcaccagcacgttcatcatcgagaagcagcctcctcaggtcctgaagacccagaccaagtttgcggccactgtgcgcctgctggtgggggggaagctgaatgtgcacatgaaccccccgcaggtgaaggcgaccatcatcagcgagcagcaggccaagtccctgctcaagaatgagaacacccgcaatgattacagcggcgagatcctgaacaactgttgcgtcatggagtaccaccaggccactggcacgctcagcgcccacttcagaaacatgtccctgaaacgaatcaagagatctgaccgccgtggtgcagagtcagtaacggaagagaagttcacgatcctgtttgactcacagttcagcgtcggtggaaacgagctggtctttcaagtcaagaccttgtcgctcccggtggtggtgattgttcacggcagccaggacaacaatgccacagccactgtcctctgggacaacgcctttgcagagcctggcagggtgccatttgccgtgcctgacaaggtgctgtggccgcagctgtgtgaagcgctcaacatgaaattcaaggctgaagtacagagcaaccggggcttgaccaaggagaacctcgtgttcctggcacagaaactgttcaacatcagcagcaaccacctcgaggactacaacagcatgtccgtgtcctggtcccagttcaaccgggagaatttgccaggacggaattacactttctggcagtggtttgatggcgtgatggaagtattgaaaaaacatctcaagcctcactggaatgatggggctatcctgggtttcgtgaacaagcaacaggcccacgacctgctcatcaacaagccggacgggaccttcctgctgcgcttcagcgactcggaaatcgggggcatcaccattgcttggaagtttgactctcaggagagaatgttttggaatctgatgccttttaccactagagacttctctatccggtccctcgctgaccgcctgggggacctgaattacctcatatatgtgtttcctgatcggccaaaggatgaagtatattctaagtactacacaccggtcccctgtgagcccgcaactgcgaaagcagctgacggatacgtgaagccacagatcaagcaggtggtccccgagtttgcaaatgcatccacagatgctgggagtggcgccacctacatggatcaggctccttccccagtcgtgtgccctcaggctcactacaacatgtacccacccaacccggactccgtccttgataccgatggggacttcgatctggaagacacgatggacgtggcgcggcgcgtggaagagctcttaggccggcccatggacagtcagtggatccctcacgcacagtcatga

SEQ ID NO: 8 - protein, 786 aa, Mus musculus
MAMWIQAQQLQGDALHQMQALYGQHFPIEVRHYLSQWIESQAWDSIDLDNPQENIKATQLLEGLVQELQKKAEHQVGEDGFLLKIKLGHYATQLQSTYDRCPMELVRCIRHILYNEQRLVREANNGSSPAGSLADAMSQKHLQINQTFEELRLITQDTENELKKLQQTQEYFIIQYQESLRIQAQFAQLGQLNPQERMSRETALQQKQVSLETWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFDSQFSVGGNELVFQVKTLSLPVVVIVHGSQDNNATATVLWDNAFAEPGRVPFAVPDKVLWPQLCEALNMKFKAEVQSNRGLTKENLVFLAQKLFNISSNHLEDYNSMSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCEPATAKAADGYVKPQIKQVVPEFANASTDAGSGATYMDQAPSPVVCPQAHYNMYPPNPDSVLDTDGDFDLEDTMDVARRVEELLGRPMDSQWIPHAQS

SEQ ID NO: 9 - DNA, 2382 bp, Artificial Sequence
  activated STAT5A
atggcgggctggattcaggcccagcagcttcagggagatgccctgcgccagatgcaagtgttgtatgggcagcatttccccatcgaggtccggcactacctggcccagtggatcgagagccagccgtgggatgctattgacttggataatccccaggaccgaggtcaggccacccaactcctggagggcctggtgcaggagctgcagaagaaggcggagcaccaggtgggggaagatgggtttttgctgaagatcaagctggggcactatgccacacagctccagaacacgtatgaccgctgtcccatggagctggttcgctgtatccgtcacattctgtacaacgaacagaggctggttcgcgaagccaacaattgcagctcccctgctggtgtcctggttgacgccatgtcccagaagcaccttcagatcaaccaaaggtttgaggagctgcgcctgatcacacaggacacggagaacgagctgaagaagctgcagcagacccaagagtacttcatcatccagtaccaggagagcctgcggatccaagctcagtttgcccagctgggccagctgaacccccaggagcgcatgagcagggagacggccctccagcagaagcaagtgtccctggagacctggctgcagcgagaggcacagacactgcagcagtaccgagtggagctggctgagaagcaccagaagaccctgcagctgctgcggaagcagcagaccatcatcctggacgacgagctgatccagtggaagcggagacagcagctggccgggaacgggggtccccccgagggcagcctggacgtgctgcagtcctggtgtgagaagctggccgagatcatctggcagaaccggcagcagatccgcagggctgagcgcctgtgccagcagctgcccatcccaggccccgtggaggagatgctggctgaggtcaacgccaccatcacggacatcatctcagctctggtcaccagcacgttcatcatcgagaagcagcctcctcaggtcctgaagacccagaccaagtttgcggccaccgtgcgcctgctggtggggggaaagctgaatgtgcacatgaaccccccgcaggtgaaggcgaccatcatcagcgagcagcaggccaagtccctgctcaagaatgagaacacccgcaatgagtgcagcggcgagatcctgaacaactgttgcgtcatggagtaccaccaggccactggcacgctcagcgcccacttcagaaacatgtcactgaaaagaatcaagcgcgccgacaggcgtggtgcagagtcggtgacggaggagaagttcacagtcctgtttgagtctcagttcagcgttggcagcaacgagctggtgttccaggtgaagaccctgtccctccctgtggtcgttatcgtccatggcagccaggaccacaatgctactgccaccgtgctgtgggacaatgcctttgctgagccgggcagggtgccatttgctgtgcctgacaaggtgctgtggccgcagctgtgtgaagcgctcaacatgaaattcaaggctgaagtacagagcaaccggggcttgaccaaagagaacctcgtgttcctggcacagaaactgttcaacatcagcagcaaccacctcgaggactacaacagcatgtctgtgtcctggtcccagttcaaccgggagaacttgcccggctggaactacaccttctggcagtggttcgacggggtgatggaggtgctgaagaagcaccataagccccattggaatgatggggctatcctgggtttcgtgaacaagcaacaggcccacgacctgctcatcaacaagccggacgggaccttcctgctgcgcttcagtgactcggaaatcgggggcatcaccattgcttggaagtttgactctccggaccgaaacctctggaatctgaagccattcacgacgcgagatttctccattcggtccctggccgaccggctgggggacctgaactaccttatctacgtgttcccagaccgacccaaggacgaggtctttgccaagtattacactcctgtacttgcgaaagcagttgacggatacgtgaagccacagatcaagcaagtggtccctgagttcgtcaatgcattcacagatgccggagccagcgccacctacatggaccaggctccttccccagtcgtgtgccctcaacctcactacaacatgtacccacccaaccctgaccctgtccttgaccaagatggcgagtttgacctggatgagagcatggatgttgccaggcacgtggaagaacttttacgccggcccatggacagtctcgacgcccgcctctccccacctgctggtctcttcacctccgctagaagctccctgtcctga

SEQ ID NO: 10 - protein, 793 aa, Artificial Sequence
  activated STAT5A
MAGWIQAQQLQGDALRQMQVLYGQHFPIEVRHYLAQWIESQPWDAIDLDNPQDRGQATQLLEGLVQELQKKAEHQVGEDGFLLKIKLGHYATQLQNTYDRCPMELVRCIRHILYNEQRLVREANNCSSPAGVLVDAMSQKHLQINQRFEELRLITQDTENELKKLQQTQEYFIIQYQESLRIQAQFAQLGQLNPQERMSRETALQQKQVSLETWLQREAQTLQQYRVELAEKHQKTLQLLRKQQTIILDDELIQWKRRQQLAGNGGPPEGSLDVLQSWCEKLAEIIWQNRQQIRRAERLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWDNAFAEPGRVPFAVPDKVLWPQLCEALNMKFKAEVQSNRGLTKENLVFLAQKLFNISSNHLEDYNSMSVSWSQFNRENLPGWNYTFWQWFDGVMEVLKKHHKPHWNDGAILGFVNKQQAHDLLINKPDGTFLLRFSDSEIGGITIAWKFDSPDRNLWNLKPFTTRDFSIRSLADRLGDLNYLIYVFPDRPKDEVFAKYYTPVLAKAVDGYVKPQIKQVVPEFVNAFTDAGASATYMDQAPSPVVCPQPHYNMYPPNPDPVLDQDGEFDLDESMDVARHVEELLRRPMDSLDARLSPPAGLFTSARSSLS

SEQ ID NO: 11 - DNA, 25 bp, Artificial Sequence
  consensus
gatccgaattccaggaattcagatc

SEQ ID NO: 12 - DNA, 2382 bp, Artificial Sequence
  activated STAT5A
atggcgggctggattcaggcccagcagcttcagggagatgccctgcgccagatgcaagtgttgtatgggcagcatttccccatcgaggtccggcactacctggcccagtggatcgagagccagccgtgggatgctattgacttggataatccccaggaccgaggtcaggccacccaactcctggagggcctggtgcaggagctgcagaagaaggcggagcaccaggtgggggaagatgggtttttgctgaagatcaagctggggcactatgccacacagctccagaacacgtatgaccgctgtcccatggagctggttcgctgtatccgtcacattctgtacaacgaacagaggctggttcgcgaagccaacaattgcagctcccctgctggtgtcctggttgacgccatgtcccagaagcaccttcagatcaaccaaaggtttgaggggctgcgcctgatcacacaggacacggagaacgagctgaagaagctgcagcagacccaagagtacttcatcatccagtaccaggagagcctgcggatccaagctcagtttgcccagctgggccagctgaacccccaggagcgcatgagcagggagacggccctccagcagaagcaagtgtccctggagacctggctgcagcgagaggcacagacactgcagcagtaccgagtggagctggctgagaagcaccagaagaccctgcagctgctgcggaagcagcagaccatcatcctggacgacgagctgatccagtggaagcggagacagcagctggccgggaacgggggtccccccgagggcagcctggacgtgctgcagtcctggtgtgagaagctggccgagatcatctggcagaaccggcagcagatccgcagggctgagcacctgtgccagcagctgcccatcccaggccccgtggaggagatgctggctgaggtcaacgccaccatcacggacatcatctcagctctggtcaccagcacgttcatcatcgagaagcagcctcctcaggtcctgaagacccagaccaagtttgcggccaccgtgcgcctgctggtggggggaaagctgaatgtgcacatgaaccccccgcaggtgaaggcgaccatcatcagcgagcagcaggccaagtccctgctcaagaatgagaacacccgcaatgagtgcagcggcgagatcctgaacaactgttgcgtcatggagtaccaccaggccactggcacgctcagcgcccacttcagaaacatgtcactgaaaagaatcaagcgcgccgacaggcgtggtgcagagtcggtgacggaggagaagttcacagtcctgtttgagtctcagttcagcgttggcagcaacgagctggtgttccaggtgaagaccctgtccctccctgtggtcgttatcgtccatggcagccaggaccacaatgctactgccaccgtgctgtgggacaatgcctttgctgagccgggcagggtgccatttgctgtgcctgacaaggtgctgtggccgcagctgtgtgaagcgctcaacatgaaattcaaggctgaagtacagagcaaccggggcttgaccaaagagaacctcgtgttcctggcacagaaactgttcaacatcagcagcaaccacctcgaggactacaacagcatgtctgtgtcctggtcccagttcaaccgggagaacttgcccggctggaactacaccttctggcagtggttcgacggggtgatggaggtgctgaagaagcaccataagccccattggaatgatggggctatcctgggtttcgtgaacaagcaacaggcccacgacctgctcatcaacaagccggacgggaccttcctgctgcgcttcagtgactcggaaatcgggggcatcaccattgcttggaagtttgactctccggaccgaaacctctggaatctgaagccattcacgacgcgagatttctccattcggtccctggccgaccggctgggggacctgaactaccttatctacgtgttcccagaccgacccaaggacgaggtctttgccaagtattacactcctgtacttgcgaaagcagttgacggatacgtgaagccacagatcaagcaagtggtccctgagttcgtcaatgcattcacagatgccggagccagcgccacctacatggaccaggctccttccccagtcgtgtgccctcaacctcactacaacatgtacccacccaaccctgaccctgtccttgaccaagatggcgagtttgacctggatgagagcatggatgttgccaggcacgtggaagaacttttacgccggcccatggacagtctcgacgcccgcctctccccacctgctggtctcttcacctccgctagaagctccctgtcctga

SEQ ID NO: 13 - protein, 793 aa, Artificial Sequence
  activated STAT5A
MAGWIQAQQLQGDALRQMQVLYGQHFPIEVRHYLAQWIESQPWDAIDLDNPQDRGQATQLLEGLVQELQKKAEHQVGEDGFLLKIKLGHYATQLQNTYDRCPMELVRCIRHILYNEQRLVREANNCSSPAGVLVDAMSQKHLQINQRFEGLRLITQDTENELKKLQQTQEYFIIQYQESLRIQAQFAQLGQLNPQERMSRETALQQKQVSLETWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWDNAFAEPGRVPFAVPDKVLWPQLCEALNMKFKAEVQSNRGLTKENLVFLAQKLFNISSNHLEDYNSMSVSWSQFNRENLPGWNYTFWQWFDGVMEVLKKHHKPHWNDGAILGFVNKQQAHDLLINKPDGTFLLRFSDSEIGGITIAWKFDSPDRNLWNLKPFTTRDFSIRSLADRLGDLNYLIYVFPDRPKDEVFAKYYTPVLAKAVDGYVKPQIKQVVPEFVNAFTDAGASATYMDQAPSPVVCPQPHYNMYPPNPDPVLDQDGEFDLDESMDVARHVEELLRRPMDSLDARLSPPAGLFTSARSSLS